SEQUENCE LISTING (39 sequences)

SEQ ID NO: 1 - DNA, 538 bp, Mus musculus
agatcaaggggaaaaggagaaccccatgcgggaactgcgcatccgcaagctctgcctcaatatctgcgtcggggagagcggagacagactgacccgggcagccaaggtgttggagcagctcacaggccagaccccggtgttctccaaagctagatacactgtcaggtcctttggcatccggagaaatgagaagattgctgttcactgcacagtccgcggagccaaggcagaggaaattctggagaaaggcctgaaggtgcgggagtatgagttgcggaaaaataacttctcggatactggaaactttggttttggaattcaagaacacattgacctgggcatcaaatacgacccaagcattgggatctacggcctggacttctatgtggtgctgggtaggccagggttcagcatcgcagacaagaagcgcagaacaggctgcattggggccaaacacagaatcagcaaggaggaggccatgcgctggttccagcagaagtacgatggaatcatccttcctggaaaataaacttgatcc

SEQ ID NO: 2 - protein, 175 aa, Mus musculus
DQGEKENPMRELRIRKLCLNICVGESGDRLTRAAKVLEQLTGQTPVFSKARYTVRSFGIRRNEKIAVHCTVRGAKAEEILEKGLKVREYELRKNNFSDTGNFGFGIQEHIDLGIKYDPSIGIYGLDFYVVLGRPGFSIADKKRRTGCIGAKHRISKEEAMRWFQQKYDGIILPGK

SEQ ID NO: 3 - DNA, 1228 bp, Mus musculus
acagccgcatcttcttgtgcagtgccagcctcgtcccgtagacaaaatggtgaaggtcggtgtgaacggatttggccgtattgggcgcctggtcaccagggctgccatttgcagtggcaaagtggagattgttgccatcaacgaccccttcattgacctcaactacatggtctacatgttccagtatgactccactcacggcaaattcaacggcacagtcaaggccgagaatgggaagcttgtcatcaacgggaagcccatcaccatcttccaggagcgagaccccactaacatcaaatggggtgaggccggtgctgagtatgtcgtggagtctactggtgtcttcaccaccatggagaaggccggggcccacttgaagggtggagccaaacgggtcatcatctccgccccttctgccgatgcccccatgtttgtgatgggtgtgaaccacgagaaatatgacaactcactcaagattgtcagcaatgcatcctgcaccaccaactgcttagcccccctggccaaggtcatccatgacaactttggcattgtggaagggctcatgaccacagtccatgccatcactgccacccagaagactgtggatggcccctctggaaagctgtggcgtgatggccgtggggctgcccagaacatcatccctgcatccactggtgctgccaaggctgtgggcaaggtcatcccagagctgaacgggaagctcactggcatggccttccgtgttcctacccccaatgtgtccgtcgtggatctgacgtgccgcctggagaaacctgccaagtatgatgacatcaagaaggtggtgaagcaggcatctgagggcccactgaagggcatcttgggctacactgaggaccaggttgtctcctgcgacttcaacagcaactcccactcttccaccttcgatgccggggctggcattgctctcaatgacaactttgtcaagctcatttcctggtatgacaatgaatacggctacagcaacagggtggtggacctcatggcctacatggcctccaaggagtaagaaaccctggaccacccaccccagcaaggacactgagcaagagaggccctatcccaactcggcccccaacactgagcatctccctcacaatttccatcccagacccccataataacaggaggggcctagggagccctccctactctcttgaataccatcaataaagttcgctgcacccac

SEQ ID NO: 4 - protein, 333 aa, Mus musculus
MVKVGVNGFGRIGRLVTRAAICSGKVEIVAINDPFIDLNYMVYMFQYDSTHGKFNGTVKAENGKLVINGKPITIFQERDPTNIKWGEAGAEYVVESTGVFTTMEKAGAHLKGGAKRVIISAPSADAPMFVMGVNHEKYDNSLKIVSNASCTTNCLAPLAKVIHDNFGIVEGLMTTVHAITATQKTVDGPSGKLWRDGRGAAQNIIPASTGAAKAVGKVIPELNGKLTGMAFRVPTPNVSVVDLTCRLEKPAKYDDIKKVVKQASEGPLKGILGYTEDQVVSCDFNSNSHSSTFDAGAGIALNDNFVKLISWYDNEYGYSNRVVDLMAYMASKE

SEQ ID NO: 5 - DNA, 1705 bp, Mus musculus
  misc_feature (611)..(611): n is a, c, g, or t/u
gccgcggtgagggaagtggacgcgatggccgggtccgcgtgggtgtccaaggtctctcggctgctgggtgcattccacaacacaaaacaggtgacaagaggttttgctggtggtgttcagacagtaactttaattcctggagatggaattggcccagaaatttcagcctcagtcatgaagatttttgatgctgccaaagcacctattcagtgggaggagcgcaatgtcacagcaattcaaggaccaggaggaaagtggatgatccctccagaagccaaggagtccatggataagaacaagatgggcttgaaaggcccactaaagaccccaatagccgctggccatccatctatgaatctgttgcttcgtaagacatttgacctttatgccaatgtccggccatgtgtctcaattgaaggttataaaaccccttacacggatgtaaatatcgtcaccatccgagagaacacggaaggagaatacagtggaattgagcatgtgatcgttgatggggttgtgcagagcatcaagctcatcaccgaagaagcaagcaagcgcattgcagagtttgcttcgagtacgctcggaacaaccaccggaaccacgtcacngctgtgcacaaaagctaacatcatgaggatgtcagatgggctctttctgcaaaaatgcagggaaatttgcggaagaactgtaaagacttaaatttaacgagatgtacttggatactgtngtttaaatatgggtanaaagacccntccaatttgatgttcttgtcatgccaaatttatacggagacatccttagtgatctgtgtgcaggactgattggaggtcttggggtgactccaagtggcaatattggagccaacggtgttgccatctttgaatcggttcatggaacagccccggacattgcaggcaaggacatggccaaccccacggccctcctgcttagtgctgtgatgatgcttcgccacatgggactttttgaccatgcagcaaaaatcgaggctgcatgttttgctacaattaaggatggaaagagcttaacaaaagatctgggaggcaacgcgaagtgctctgacttcacagaagaaatctgtcgtagagtcaaagacttagattagcactcctgctggtggatttgctgcagtcagtcaatcactccaaaaggataccctgtaatcctccttgagggcgcccaccattggtttgcttggttcttgacagagtacgttttttgaatctggccttttcttaacaaaaccctttgcaatggatgcacatgntggccccaggcctttcattcaaaaggtttnccccaagtgctggtggtatttattgtcccgtctgggtaaacnttattttgtaaactgtaagtgaactgtatcatttatcattgttaacccattttacacttcaggcaaaatcattttcctcaactgtaaatattctgatacagaattaataagagaagatatttaactttttaacaaaagccctggatttttggtttatgaaaaacaaactgggaataaaacagggttttaacaatcgcacaagataacattattctaatactaatgggtacaaaagaaatttactgggaaagttcacagcaaaaaaatggtatatttcttaaaaatatggaaataaagtatttgtcctatacatgaattactattaataaaaatgtaagctccaag

SEQ ID NO: 6 - protein, 233 aa, Mus musculus
  misc_feature (204)..(204): Xaa is unknown or other
AAVREVDAMAGSAWVSKVSRLLGAFHNTKQVTRGFAGGVQTVTLIPGDGIGPEISASVMKIFDAAKAPIQWEERNVTAIQGPGGKWMIPPEAKESMDKNKMGLKGPLKTPIAAGHPSMNLLLRKTFDLYANVRPCVSIEGYKTPYTDVNIVTIRENTEGEYSGIEHVIVDGVVQSIKLITEEASKRIAEFASSTLGTTTGTTSXLCTKANIMRMSDGLFLQKCREICGRTVKT

SEQ ID NO: 7 - DNA, 853 bp, Mus musculus
gccatgtttggagagagaagagccaaacagccatctccctgcacagtccttcaagctcacctcctgccttccgtggacaagaggaagcacaaagaatcatccaggtatggaagctgagggttccagccgcaaggtcaccaggctactccgcctgggagtcaaggaagactcggaagaacagcatgatgtgaaagcagaggctttcttccaggctggagaggggagagatgagcaaggtgcacagggccagcctggagtgggagcggtgggaacagaaggcgaaggagaagaattaaatggaggaaaaggccactttggtcctggtgctcctggtcctatgggtgatggggacaaggatagtggcaccagggctggtggtgtggagcaggaacaaaatgagccagttgctgagggcactgagagccaggagaatggaaatcctgggggtaggcagatgcccctccagggctctaggttcgcccagcatcgactgagggaactggagtccattttgcagcgcactaattcctttgatgtcccaagggaggatcttgatagactgatggatgcctgtgtgtccagagtgcagaattggtttaagatcaggagggctgcggcaagaagaaccaggaggagggcaacaccagtccctgaacattttagaggaacattcgagtgtcctgcttgtcgtggagtgagatggggagaaagatgcccttttgcgacaccgagattttgatttgatcacatatgccggctatgacagcccttacttttcaagaattcagcaataaagaggtggattcccagtatgtttgttccattacctctatgattattaaaatattgatac

SEQ ID NO: 8 - protein, 210 aa, Mus musculus
MEAEGSSRKVTRLLRLGVKEDSEEQHDVKAEAFFQAGEGRDEQGAQGQPGVGAVGTEGEGEELNGGKGHFGPGAPGPMGDGDKDSGTRAGGVEQEQNEPVAEGTESQENGNPGGRQMPLQGSRFAQHRLRELESILQRTNSFDVPREDLDRLMDACVSRVQNWFKIRRAAARRDRRRATPVPEHFRGTFECPACRGVRWGERCPFATPRF

SEQ ID NO: 9 - DNA, 882 bp, Mus musculus
  misc_feature (551)..(551): n is a, c, g, or t/u
ggcacgagctccggcctaggagaccgcgatggcgttcctgtgtcagcgcgacagctacgcacgggagttcaccaccaccgtggtctcctgtagtcccgccgagctgcagacggacgcgagcggcggcaagaaagaagtgttgagcggattccatgtggttctggaagacacgctgcttttccccgagggcgggggacagcctgatgaccgtggtaccatcaatgacatctctgtgctgagggtgacccgtcgtggggcccaggccgatcacttcacggagtcacctctgtcccctgggagtcaagtccaggtccgggtggactgggagcggaggtttgaccacatgcagcagcattcagggcaacatctcatcaccgcggttgctgaccttctcttcgggctgaagacaacgtcatgggagttagggagactccggagtgtgattgagttggacagcccttctgtgactgctgagcaggtggctgctatcgagcaagagtgtcaatcagaaaatcagagaccggctgcctggtgagtgttcgagagctganctgnatgaccctgaaggtggaccaggtgaagggggncggggntttgcccgatgaatcaatgctgggcccattcgagnttnttnccaatcggaaggtgttaaaacnccaacaatgtgcngtngggaacncacgtnaagccaaacctcgntgaccttcaggtcnattnaaaattctggggganctggaaaaagggnaaaaaagnaccaaaagccaacctggatatttcnnggccgggnancnggttantgaantttggattggnnacnnaattcctgggaagttgaaaaagcctttgnacccccctaacttnaaantntgngcattnaaantn

SEQ ID NO: 10 - protein, 171 aa, Mus musculus
ETAMAFLCQRDSYAREFTTTVVSCSPAELQTDASGGKKEVLSGFHVVLEDTLLFPEGGGQPDDRGTINDISVLRVTRRGAQADHFTESPLSPGSQVQVRVDWERRFDHMQQHSGQHLITAVADLLFGLKTTSWELGRLRSVIELDSPSVTAEQVAAIEQECQSENQRPAAW

SEQ ID NO: 11 - DNA, 1464 bp, Mus musculus
tcaaggacgctcaccctggtcttggggcgccaggtttctgggctgcagccgcacctggaaaggaggaatcgggagattgatgccagcataaggctggggctgcagggaaggcagggttccatctggtggaggcagcagagtgaaggcctgcagcagacgagccagcaccacaaagagctccagccgtgccagaggctctcccaggcacacgcgtgccccacagccaaaggatggtgttctgggattcttcccaggttccaggaagcgatctggccagaacttgctgggcagttcccaaaccatctcatccaggttggcgccttggatgttggggatgatgaccatatccttagggatgtcatagccggagatgctgctagccctagttgcacgatggggcaaggccaagggcaccacaggccgcaaacgcagcacctcggcaatggtggccatgagcagaggcagctgcattcggttcctgtacaggagctgggagcctgggcccagcttgaggtctaactcttcctgcagtcgcttctggatctcagggtggtgaagcaggaaagccacagcccaggagagcgtggtagccgtggtctcggtgccgccgatgaacaggtccaccaccgacatgtgcacgtgcccctcgtggagccgctcttcgtctttgccatccctttgcttctccactccctggagcatgtagtcaatcatgtctttccattggcctgcaaccaggctgtccttgtgccgcttcagctgctgcttgacaatatggtcccgactctcttggatctgcttcagcttctggaggcctgggttggggaggaacctgagaaggggaattatcgtcaagatttggatggaccagtggttccaggcttgcaacaagtcctggacacagtcgtgaagggtctgtaccaacgtgctgtccttgtctccaaaagtgaggcaggagatgatactacaagtgaggaaggagaattccttatggatggccacgggggtgccagcctgggctcgcatgcgctcacagaactcctgggtcagctgctctatcagaggctccatggagtctcgcatgcccagcatcagggctgagcgagagagtttcttgtgggccttccacatgagagagtaatcccccagggacaggtccaagtccatctttccatttagcatatggggtcggccagcaaagtccacccacttttggatcaaggcctcctcaatggttctgttagaatttagcaccaccacatcttgcatccccaagcggatcctgtagatgggcccgagtttctgagtgaggccaagcaggtagatgggaaggttaggctgtaggaagtgcagaaaacccggggccagaggcgggaggtgcagcttccggagcttccattggccccacagccagcgggtgccagctagcagcagcaacagcagcagcagcccaggtagcagcat

SEQ ID NO: 12 - protein, 487 aa, Mus musculus
MLLPGLLLLLLLLAGTRWLWGQWKLRKLHLPPLAPGFLHFLQPNLPIYLLGLTQKLGPIYRIRLGMQDVVVLNSNRTIEEALIQKWVDFAGRPHMLNGKMDLDLSLGDYSLMWKAHKKLSRSALMLGMRDSMEPLIEQLTQEFCERMRAQAGTPVAIHKEFSFLTCSIISCLTFGDKDSTLVQTLHDCVQDLLQAWNHWSIQILTIIPLLRFLPNPGLQKLKQIQESRDHIVKQQLKRHKDSLVAGQWKDMIDYMLQGVEKQRDGKDEERLHEGHVHMSVVDLFIGGTETTATTLSWAVAFLLHHPEIQKRLQEELDLKLGPGSQLLYRNRMQLPLLMATIAEVLRLRPVVPLALPHRATRASSISGYDIPKDMVIIPNIQGANLDEMVWELPSKFWPDRFLEPGKNPRTPSFGCGARVCLGEPLARLELFVVLARLLQAFTLLPPPDGTLPSLQPQPYAGINLPIPPFQVRLQPRNLAPQDQGERP

SEQ ID NO: 13 - DNA, 985 bp, Mus musculus
ggcacgagcaacagctgaagaggctcccacagaaaccgctgttgaagagtcgggtgaagctggagaggacggcgctcctgagggtatggcagagactgggggaagaggcagcgggaagaaggaggctgggtccagcaagagcaccgtggacgggaagctggtaaaagcttcttttgctccaataagctttgccatcaaagccatgaaagaaggacggtacacggtgctagctgagagcaagaacgaggagaagaaaaagtctgggccaacctctgacaacgaagaggaagatgatgaggaagatgggagttacctgcacccgtctctctttgcttccaagaagagcagccgcctggaagagctgatgaagcccttgaaggtggtggacccggatcaccctctagcagccctttgtccggaaagcacaaagctgacagctcactcccagccccaccccaacttgcaagaatggggcccaagaacgatcaaagatttgatttctttcagccctggcacccagtacaacgcctactacgagttttaaagaagcagttcttcctgcagaaagaaggaggcggtagcacacaggcagcatcaacagctgaagaggctcccacagaaaccgctgttgaagagtcgggaaagcagctcgccatcaaccagacatgtccgaaggcgggaatccagccaggagctctccttccgcaatgaagacattggagtcatgggacctctcagcggccctgtccgtcctgatccagcggtaatgggaacacgcctggagctgtcctgtgattcattgcccccctccattactgctctgagcctattcatgtcagcaccatctgtctaaaatcactacccaggaatgctttttaacgtcatgatcacatctaaatgaggtcactctgccagcctgaggcttctaggtctgcagaggaaggaagccttttctctgcagaaaataaaaaggaccatgtgcaat

SEQ ID NO: 14 - protein, 180 aa, Mus musculus
ARATAEEAPTETAVEESGEAGEDGAPEGMAETGGRGSGKKEAGSSKSTVDGKLVKASFAPISFAIKAMKEGRYTVLAESKNEEKKKSGPTSDNEEEDDEEDGSYLHPSLFASKKSSRLEELMKPLKVVDPDHPLAALCPESTKLTAHSQPHPNLQEWGPRTIKDLISFSPGTQYNAYYEF

SEQ ID NO: 15 - DNA, 612 bp, Mus musculus
  misc_feature (77)..(77-): n is a, c, g, or t/u
cggcacgagctcgtgtcctctgcgcctgtgtccgccgcctgcccgcggccttcgcgccgctgccccggcttcccacnttggccttggcgcggccgctcagcaccaccctatgccctgagggaatccggaggagacccggggctctgcagtccgccttggcgctcgcgcaggtgcctggaacagtcacacatttgtgccgccagtacagtgacgcacccccactgacgttagaggaatcaaggaccgagttctgtatgtcttgaaactctatgataagattgatccagaaaagctctccgtaaattctcattttatgaaggacctgggcttagacagtttggaccaagtggaaattattatggccatggaagacgaatttgggtttgaaattcctgatatagatgcagagaagttaatgtgtccacaagaaattgtagattacattgcagataagaaggatgtgtatgaataaagtatcagagccttcttcctcactgtgaggactccagaggacacacgatggcatcgtggccgactgacagcggctctgttcaacttgtatttaaattgtctgagtgttttacccgttaaaaataaatctattacaaaact

SEQ ID NO: 16 - protein, 86 aa, Mus musculus
RTPTDVRGIKDRVLYVLKLYDKIDPEKLSVNSHFMKDLGLDSLDQVEIIMAMEDEFGFEIPDIDAEKLMCPQEIVDYIADKKDVYE

SEQ ID NO: 17 - DNA, 1036 bp, Mus musculus
ggcacgagggaggagggggctgggcctgttgaccgactgccagtgagagggaaagctggcaagtttaaggatgatcccgagaagggggcaaggtcttcccgctttactagtgttaaccatgatgcgaaggaagagtgtggcaaggtagaatcaccccctgcagcgaggtgctcggctcgcagagctgagctctcgaagcagaatggctcctcggcctctcagatttcttctgctgaaggcagggcagctgcaaaaggtaacaacagcttggagagggagaggcagaatttaccaggagcccttgttctaaacttacaatgaaaccagtcagtcaattagactaaagttgttgattccttgtgattatttccatgtgaaaatggttgtgtacaatgacatttaaaaaaaatcatcctctcgtttagaaggtagaaaggggggaaaggaaactttctaaatgctgcttgagattgcagtaagaacatacattttctaacctgaaagttgaaacaaatcccacttgttctgtagactgtgtctctcttacctgttgctgtcagggttaccttatctgctaaactatgtcgggaaagaaaaaattacttttgtttgcatgtcatgggttaatggtccctgtaatttggcagtgggtgtaaaagcttattaaagttcttcttttgctttgacccagaacaatggcatcatttggatttttgtctgaaatcgtgataccaggtaactccaaattgatcccttgcatttgcaacaaaagtattgtgtttcagtgttctcacctgtagaaaactagttttcactagaaatgctcatcagaacaccaaaaaaaaaaccatctttaataggaataaggtgtataattgcttgttgtacagaaatggttgactaaagagagagaaacaaagcgtgggaaatttaaaaaaaaaacccacagagaaacaatggtaaaaaatgaatccaaagagtacgggtgagcaagtacaaatcacctttgagaaaacagaaactgtcagaatgggtg

SEQ ID NO: 18 - protein, 106 aa, Mus musculus
GTREEGAGPVDRLPVRGKAGKFKDDPEKGARSSRFTSVNHDAKEECGKVESPPAARCSARRAELSKQNGSSASQISSAEGRAAAKGNNSLERERQNLPGALVLNLQ

SEQ ID NO: 19 - DNA, 530 bp, Mus musculus
ggcacgaggaagtgaaaaggccttgacctcactacttaagtgtggcgttgaggatcatgtggaagcagtaaaaaagctccagaatgccaccaagctcctgcagaagaacaacctgaacctccttagagacctggctgtgcacactgcccacagcctcaggagcagcccagcctggggtggtgtggtcacactacacaggaaagagggtgattctgaattcatgaatatcattgctaatgagattggatcggaggagaccctcctgttcttaactgtgggggatgagaagggtgctgggctcttcttactggcaggcccggcagaggctgtggaaaccctggggcccagggtggctgaagtcttggaaggcaaaggagcagggaagaagggccgcttccagggcaaagccaccaagatgagccgccgggcagaggcgcaggcgcttctgcaggactatgtcagcacacagagtgctgaggagtgaggggccaggactcgtcctgtgaccaacagttaaaatattgtgactc

SEQ ID NO: 20 - protein, 160 aa, Mus musculus
ARGSEKALTSLLKCGVEDHVEAVKKLQNATKLLQKNNLNLLRDLAVHTAHSLRSSPAWGGVVTLHRKEGDSEFMNIIANEIGSEETLLFLTVGDEKGAGLFLLAGPAEAVETLGPRVAEVLEGKGAGKKGRFQGKATKMSRRAEAQALLQDYVSTQSAEE

SEQ ID NO: 21 - DNA, 20 bp, Mus musculus
gtggacaagaggaagcacaa

SEQ ID NO: 22 - DNA, 20 bp, Mus musculus
tgaaaagtaagggctgtcat

SEQ ID NO: 23 - DNA, 1895 bp, Homo sapiens
  CDS (49)...(1677)
gttagaggcggcttgtgtccacgggacgcgggcggatcttctccggccatgaggaagccagccgctggcttccttccctcactcctgaaggtgctgctcctgcctctggcacctgccgcagcccaggattcgactcaggcccccactccaggcagccctctctctcctaccgaatacgaacgcttcttcgcactgctgactccaacctggaaggcagagactacctgccgtctccgtgcaacccacggctgccggaatcccacactcgtccagctggaccaatatgaaaaccacggcttagtgcccgatggtgctgtctgctccaacctcccttatgcctcctggtttgagtctttctgccagttcactcactaccgttgctccaaccacgtctactatgccaagagagtcctgtgttcccagccagtctctattctctcacctaacactctcaaggagatagaagcttcagctgaagtctcacccaccacgatgacctcccccatctcaccccacttcacagtgacagaacgccagaccttccagccctggcctgagaggctcagcaacaacgtggaagagctcctacaatcctccttgtccctgggaggccaggagcaagcgccagagcacaagcaggagcaaggagtggagcacaggcaggagccgacacaagaacacaagcaggaagaggggcagaaacaggaagagcaagaagaggaacaggaagaggagggaaagcaggaagaaggacaggggactaaggagggacgggaggctgtgtctcagctgcagacagactcagagcccaagtttcactctgaatctctatcttctaacccttcctcttttgctccccgggtacgagaagtagagtctactcctatgataatggagaacatccaggagctcattcgatcagcccaggaaatagatgaaatgaatgaaatatatgatgagaactcctactggagaaaccaaaaccctggcagcttcctgcagctgccccacacagaggccttgctggtgctgtgctattcgatcgtggagaatacctgcatcataacccccacagccaaggcctggaagtacatggaggaggagatccttggtttcgggaagtcggtctgtgacagccttgggcggcgacacatgtctacctgtgccctctgtgacttctgctccttgaagctggagcagtgccactcagaggccagcctgcagcggcaacaatgcgacacctcccacaagactccctttgtcagccccttgcttgcctcccagagcctgtccatcggcaaccaggtagggtccccagaatcaggccgcttttacgggctggatttgtacggtgggctccacatggacttctggtgtgcccggcttgccacgaaaggctgtgaagatgtccgagtctctgggtggctccagactgagttccttagcttccaggatggggatttccctaccaagatttgtgacacagactatatccagtacccaaactactgttccttcaaaagccagcagtgtctgatgagaaaccgcaatcggaaggtgtcccgcatgagatgtctgcagaatgagacttacagtgcgctgagccctggcaaaagtgaggacgttgtgcttcgatggagccaggagttcagcaccttgactctaggccagttcggatgagctggcgtctattctgcccacaccccagcccaacctgcccacgttctctattgttttgagaccccattgctttcaggctgccccttctgggtctgttactcggcccctactcacatttccttgggttggagcaacagtcccagagagggccacggtgggagctgcgccctccttaaaagatgactttacataaaatgttgatcttcaaaaaaaaa

SEQ ID NO: 24 - protein, 543 aa, Homo sapiens [sequence omitted]

SEQ ID NO: 25 - DNA, 20 bp, Mus musculus
gtggacaagaggaagcacaa

SEQ ID NO: 26 - DNA, 19 bp, Homo sapiens
tctccccatctcactccac

SEQ ID NO: 27 - DNA, 20 bp, Homo sapiens
aaggacaggggactaaggag

SEQ ID NO: 28 - DNA, 20 bp, Homo sapiens
ccgtacaaatccagcccgta

SEQ ID NO: 29 - DNA, 20 bp, Homo sapiens
atgtgagtaggggccgagta

SEQ ID NO: 30 - DNA, 21 bp, Homo sapiens
ttcctgggctgatcgaatgag

SEQ ID NO: 31 - DNA, 22 bp, Homo sapiens
gcaaaagaggaagggttagaag

SEQ ID NO: 32 - DNA, 20 bp, Homo sapiens
ccgtggttttcatattggtc

SEQ ID NO: 33 - DNA, 19 bp, Homo sapiens
ctggcgtctattctgccca

SEQ ID NO: 34 - DNA, 23 bp, Homo sapiens
tgtaaagtcatcttttaaggagg

SEQ ID NO: 35 - DNA, 16 bp, Homo sapiens
ccccatctcaccccac

SEQ ID NO: 36 - DNA, 9 bp, Homo sapiens
cttcgtgtt

SEQ ID NO: 37 - protein, 543 aa, Cavia porcellus
MGQPAAGSILTLLRVLLLPLGPALAQDSPSAPTPGSPLSPTEYERFFALLTPTWKAETTCRLRATHGCRNPTIVQLDQYENHGLVPDGAVCSDLPYASWFESFCQFSQYRCSNHVYYAKRVRCSQPVSILSVNSFKELESPVEVSPTTMTSPVTSHIKATERQSFQAWPERLSNNVEELLQSSLSLAGQEQAAGHKQEQGQEQHKQDPTQEHKQDDGQEQEEQEEEQEEEGKQEEGQSVEDMLGRVGRAGLRIGSEPKPQSLSLSSDPHSFTARVRDVDSAPMMIENIQELIQSAQEMEEMYEEDAYWRSQNHGSLLQLPHKEALLVLCYSIVMNSCVMTPSAKAWKYLEEETFGFGKSVCDNLGRRHMALCPLCAFCSLKLEQCHSEANLQRQQCDASHKTPFISSLLTAQTMSMGTQAGTSESGRFYGLDVYGGLRMDFWCARLATKGCEDIRVSSWLQTEFLSFHNGDFPTKVCDTDYIQYPNYCSFKSQQCLMKNRNRKVSRMRCMQNETYNVLTPSKGEDLVLRWSQEFSTLALSRFG

SEQ ID NO: 38 - protein, 537 aa, Sus Scrofa
QLAAGSLLSLLKVLLLPLAPAPAQDANSASTPGSPLSPTEYERFFALLTPTWKAETTCRLRATHGCRNPTLVQLDQYENHGLVPDGAVCSDLPYASWFESFCQFTQYRCSNHVYYAKRVRCSQPVSILSPNSLKEVDTSSEVPITTMTSPVSSHITATGRQVFQPWPERLNNNVEELLQSSLSLGGQEQGQEHKQEHKQEQGQEHKQDEGQEQEEQEEEQEEEGKQEEGQGTEESLEAMSGLQADSEPKFQSEFVSSNPFSFTPRVREVESTPMMMENIQELIRSAQEMDEMGDVYEEENIWRAQSPGSLLQLPHVDALLVLCYSIVENTCVITPTAKAWQYLEDETLGFGKSVCDSLGRRHLAACSLCDFCSLKLEQCHSETNLQRQQCDNSHKTPFISPLLASQSMSIGTQIGTLKSGRFYGLDLYGGLRMDFWCARLATKGCEDNRVASWLQTEFLSFQDGDFPTKICDTEYVQYPNYCAFKSQQCMMRNRDRKVSRMRCLQNETYTVLTQAKSEDLVLRWSQEFSTLTLGQAG

SEQ ID NO: 39 - protein, 540 aa, Mus musculus
MMNLAAGFLLMLLEVLLLPGTPLSAEESPASTPGSPLSSTEYERFFALLTPTWKAETTCRLRATHGCRNPTLVQLDQYENHGLVPDGAVCSDLPYASWFESFCQFAQYRCSNHVYYAKRVRCSQPVSILSPNTLKEVESSAEVPPTSMTTPIVSHATATEHQAFQPWPERLNNNVEELLQSSLSLGGKDQQSSRRPGQEQHKQEQIQEHKLEEAQEQEEQEEEEEEEEAKQEEGQGTEAGLESVSRLQSDSEPKFQSQSLSSNPSFFTPRVREVESAPLMMKNIQELIRSAQEMDEMNELYDDSWRSQSTGSLQQLPHMETLMVLCYSIMENTCTMTPTAKAWSYMEEEILGFGDSVCDNLGRRHTAACPLCAFCSLKLEQCHSEASVVRQKCDASHKIPFISPLLSAQSISTGNQARIPDKGRFAGLEMYGGLSSEFWCNRLAMKGCEDDRVSNWLKAEFLSFQEGDFPTKICDTNYIQYPNYCSFKSQQCLLRNQNRKMSRMRCMLNERYNVLSLAKSEEVILRWSQEFSTLAIGQFG

(19) United States
(12) Patent Application Publication
(10) Pub. No.: US 2004/0086852 A1
(43) Pub. Date: May 6, 2004

(54) Cancer associated antigens and uses therefor

(76) Inventors: Toshiro Ono, Okayama (JP); Eiichi Nakayama, Okayama (JP)

Correspondence Address: WOLF GREENFIELD & SACKS, PC, FEDERAL RESERVE PLAZA, 600 ATLANTIC AVENUE, BOSTON, MA, 02210-2211, US

(21) Appl. No.: 10/148,641

(22) PCT Filed: Dec. 1, 2000
(86) PCT No.: PCT/US00/32750

(51) Int. Cl. C12Q 1/68; G01N 33/574

(52) U.S. Cl. 435/6; 435/7.23

(57) ABSTRACT

Cancer associated antigens have been identified by autologous antibody screening of libraries of nucleic acids expressed in methylcholanthrene-induced fibrosarcoma cancer cells using antisera from mice bearing such tumors. The invention relates to nucleic acids and encoded polypeptides which are cancer associated antigens expressed in mice afflicted with methylcholanthrene-induced fibrosarcomas, as well as homologs thereof, particularly human homologs. The invention provides, inter alia, isolated nucleic acid molecules, expression vectors containing those molecules and host cells transfected with those molecules. The invention also provides isolated proteins and peptides, antibodies to those proteins and peptides and cytotoxic T lymphocytes which recognize the proteins and peptides. Fragments of the foregoing including functional fragments and variants also are provided. Kits containing the foregoing molecules additionally are provided. The molecules provided by the invention can be used in the diagnosis, monitoring, research, or treatment of conditions characterized by the expression of one or more cancer associated antigens.

### FIELD OF THE INVENTION

The invention relates to nucleic acids and encoded polypeptides which are cancer associated antigens expressed in methylcholanthrene-induced fibrosarcomas. The invention also relates to agents which bind the nucleic acids or polypeptides. The nucleic acid molecules, polypeptides coded for by such molecules and peptides derived therefrom, as well as related antibodies and cytolytic T lymphocytes, are useful, inter alia, in diagnostic and therapeutic contexts.

### BACKGROUND OF THE INVENTION

The mechanism by which T cells recognize foreign materials has been implicated in cancer. A number of cytolytic T lymphocyte (CTL) clones directed against autologous melanoma antigens, testicular antigens, and melanocyte differentiation antigens have been described. In many instances, the antigens recognized by these clones have been characterized.

The use of autologous CTLs for identifying tumor antigens requires that the target cells which express the antigens can be cultured in vitro and that stable lines of autologous CTL clones which recognize the antigen-expressing cells can be isolated and propagated. While this approach has worked well for melanoma antigens, other tumor types, such as epithelial cancers including breast and colon cancer, have proved refractory to the approach.

More recently another approach to the problem has been described by Sahin et al. (Proc. Natl. Acad. Sci. USA 92:11810-11813, 1995). According to this approach, autologous antisera are used to identify immunogenic protein antigens expressed in cancer cells by screening expression libraries constructed from tumor cell cDNA. Antigen-encoding clones so identified have been found to have elicited an high-titer humoral immune response in the patients from which the antisera were obtained. Such a high-titer IgG response implies helper T cell recognition of the detected antigen. These tumor antigens can then be screened for the presence of MHC/HLA class I and class II motifs and reactivity with CTLs.

Presently there is a need for additional cancer antigens for development of therapeutics and diagnosis applicable to a greater number of cancer patients having various cancers.

### SUMMARY OF THE INVENTION

Autologous antibody screening has now been applied to methylcholanthrene-induced fibrosarcomas using antisera from mice bearing such tumors. Numerous cancer associated antigens have been identified, including human nucleic acid homologs of mouse clones so identified. The invention provides, inter alia, isolated nucleic acid molecules, expression vectors containing those molecules and host cells transfected with those molecules. The invention also provides isolated proteins and peptides, antibodies to those proteins and peptides and CTLs which recognize the proteins and peptides. Fragments including functional fragments and variants of the foregoing also are provided. Kits containing the foregoing molecules additionally are provided. The foregoing can be used in the diagnosis, monitoring, research, or treatment of conditions characterized by the expression of one or more cancer associated antigens.

The invention involves the surprising discovery of several genes, some previously known and some previously unknown, which are expressed in mice bearing methylcholanthrene-induced fibrosarcomas. These individuals all have serum antibodies against the proteins (or fragments thereof) encoded by these genes. Thus, abnormally expressed genes are recognized by the host's immune system and therefore can form a basis for diagnosis, monitoring and therapy. The human equivalents (i.e., homologs) of the nucleic acids have in some cases also been identified.

The invention involves the use of a single material, a plurality of different materials and even large panels and combinations of materials. For example, a single gene, a single protein encoded by a gene, a single functional fragment thereof, a single antibody thereto, etc. can be used in methods and products of the invention. Likewise, pairs, groups and even panels of these materials and optionally other cancer associated antigen genes and/or gene products can be used for diagnosis, monitoring and therapy. The pairs, groups or panels can involve 2, 3, 4, 5 or more genes, gene products, fragments thereof or agents that recognize such materials. A plurality of such materials are not only useful in monitoring, typing, characterizing and diagnosing cells abnormally expressing such genes, but a plurality of such materials can be used therapeutically. An example of the use of a plurality of such materials for the prevention, delay of onset, amelioration, etc. of cancer cells, which express or will express such genes prophylactically or acutely. Any and all combinations of the genes, gene products, and materials which recognize the genes and gene products can be tested and identified for use according to the invention. It would be far too lengthy to recite all such combinations; those skilled in the art, particularly in view of the teaching contained herein, will readily be able to determine which combinations are most appropriate for which circumstances.

As will be clear from the following discussion, the invention has in vivo and in vitro uses, including for therapeutic, diagnostic, monitoring and research purposes. One aspect of the invention is the ability to fingerprint a cell expressing a number of the genes identified according to the invention by, for example, quantifying the expression of such gene products. Such fingerprints will be characteristic, for example, of the stage of the cancer, the type of the cancer, or even the effect in animal models of a therapy on a cancer. Cells also can be screened to determine whether such cells abnormally express the genes identified according to the invention.

The invention, in one aspect, is a method of diagnosing a disorder characterized by expression of a cancer associated antigen precursor coded for by a nucleic acid molecule. The method involves the steps of contacting a biological sample isolated from a subject with an agent that specifically binds to the nucleic acid molecule, an expression product thereof, or a fragment of an expression product thereof complexed with a MHC, preferably an HLA, molecule, wherein the nucleic acid molecule is a NA Group 1 nucleic acid molecule, and determining the interaction between the agent and the nucleic acid molecule, the expression product or fragment of the expression product as a determination of the disorder.

In one embodiment the agent is selected from the group consisting of (a) a nucleic acid molecule comprising NA Group 1 nucleic acid molecules or a fragment thereof, (b) a nucleic acid molecule comprising NA Group 3 nucleic acid molecules or a fragment thereof,

(c) a nucleic acid molecule comprising NA Group 5 nucleic acid molecules or a fragment thereof, (d) an antibody that binds to an expression product, or a fragment thereof, of NA group 1 nucleic acids, (e) an antibody that binds to an expression product, or a fragment thereof, of NA group 3 nucleic acids, (f) an antibody that binds to an expression product, or a fragment thereof, of NA group 5 nucleic acids, (g) and agent that binds to a complex of an MHC, preferably HLA, molecule and a fragment of an expression product of a NA Group 1 nucleic acid, (h) an agent that binds to a complex of an MHC, preferably HLA, molecule and a fragment of an expression product of a NA group 3 nucleic acid, and (i) an agent that binds to a complex of an MHC, preferably HLA, molecule and a fragment of an expression product of a NA Group 5 nucleic acid.

The disorder may be characterized by expression of a plurality of cancer associated antigen precursors. Thus the methods of diagnosis may include use of a plurality of agents, each of which is specific for a different cancer associated antigen precursor (including at least one of the cancer associated antigen precursors disclosed herein), and wherein said plurality of agents is at least 2, at least 3, at least 4, at least 5, at least 6, at least 7, at least 8, at least 9 or at least 10 such agents.

In each of the above embodiments the agent may be specific for a cancer associated antigen precursor, including the cancer associated antigen precursors disclosed herein, and preferably human homologs thereof.

In another aspect the invention is a method for determining regression, progression or onset of a condition characterized by expression of abnormal levels of a protein encoded by a nucleic acid molecule that is a NA Group 1 molecule. The method involves the steps of monitoring a sample, from a subject who has or is suspected of having the condition, for a parameter selected from the group consisting of (i) the protein, (ii) a peptide-derived from the protein, (iii) an antibody which selectively binds the protein or peptide, and (iv) cytolytic T cells specific for a complex of the peptide derived from the protein and a MHC molecule, as a determination of regression, progression or onset of said condition. In one embodiment the sample is a body fluid, a body effusion or a tissue.

In another embodiment the step of monitoring comprises contacting the sample with a detectable agent selected from the group consisting of (a) an antibody which selectively binds the protein of (i), or the peptide of (ii), (b) a protein or peptide which binds the antibody of (iii), and (c) a cell which presents the complex of the peptide and MHC molecule of (iv). In a preferred embodiment the antibody, the protein, the peptide or the cell is labeled with a radioactive label or an enzyme. The sample in a preferred embodiment is assayed for the peptide.

According to another embodiment the nucleic acid molecule is one of the following: a NA Group 3 molecule or a NA Group 5 molecule. In yet another embodiment the protein is a plurality of proteins, the parameter is a plurality of parameters, each of the plurality of parameters being specific for a different of the plurality of proteins.

The invention in another aspect is a pharmaceutical preparation, preferably for a human subject. The pharmaceutical preparation includes an agent which when administered to the subject enriches selectively the presence of complexes of a MHC molecule, preferably a HLA molecule, and a cancer associated antigen, and a pharmaceutically acceptable carrier, wherein the cancer associated antigen is a fragment of a cancer associated antigen precursor encoded by a nucleic acid molecule which comprises a NA Group 1 molecule, or a human homolog thereof. In one embodiment the nucleic acid molecule is a NA Group 3 nucleic acid molecule.

The agent in one embodiment comprises a plurality of agents, each of which enriches selectively in the subject complexes of a MHC molecule and a different cancer associated antigen. Preferably the plurality is at least two, at least three, at least four or at least 5 different such agents.

In another embodiment the agent is selected from the group consisting of (1) an isolated polypeptide comprising the cancer associated antigen, a human homolog thereof, or a functional variant thereof, (2) an isolated nucleic acid operably linked to a promoter for expressing the isolated polypeptide, homolog or functional variant thereof, (3) a host cell expressing the isolated polypeptide, homolog or functional variant thereof, and (4) isolated complexes of the polypeptide, homolog or functional variants thereof, and an HLA molecule.

The agent may be a cell expressing an isolated polypeptide. In one embodiment the agent is a cell expressing an isolated polypeptide comprising the cancer associated antigen, a human homolog thereof or a functional variant thereof; In another embodiment the agent is a cell expressing an isolated polypeptide comprising the cancer associated antigen, a homolog or a functional variant thereof, and wherein the cell expresses an HLA molecule that binds the polypeptide. The cell can express one or both of the polypeptide and HLA molecule recombinantly. In preferred embodiments the cell is nonproliferative. In yet another embodiment the agent is at least two, at least three, at least four or at least five different polypeptides, each representing a different cancer associated antigen, homolog or functional variant thereof.

The agent in one embodiment is a PP Group 2 polypeptide. In other embodiments the agent is a PP Group 3 polypeptide or a PP Group 4 polypeptide.

In an embodiment each of the pharmaceutical preparations described herein also includes an adjuvant.

According to another aspect the invention, a composition is provided which includes an isolated agent that binds selectively a PP Group 1 polypeptide. In separate embodiments the agent binds selectively to a polypeptide selected from the following: a PP Group 2 polypeptide, a PP Group 3 polypeptide, a PP Group 4 polypeptide, and a PP Group 5 polypeptide. In other embodiments, the agent is a plurality of different agents that bind selectively at least two, at least three, at least four, or at least five different such polypeptides. In each of the above described embodiments the agent may be an antibody.

In another aspect the invention is a composition of matter composed of a conjugate of the agent of the above-described compositions of the invention and a therapeutic or diagnostic agent. Preferably the conjugate is of the agent and a therapeutic or diagnostic that is an antineoplastic:

The invention in another aspect is a pharmaceutical composition which includes an isolated nucleic acid molecule selected from the group consisting of: (I) NA Group 1 molecules, and (2) NA Group 2 molecules, and a pharmaceutically acceptable carrier. In one embodiment the isolated nucleic acid molecule comprises a NA Group 3 or NA Group 4 molecule. In another embodiment the isolated nucleic acid molecule comprises at least two isolated nucleic acid molecules coding for two different polypeptides, each polypeptide comprising a different cancer associated antigen.

Preferably the pharmaceutical composition also includes an expression vector with a promoter operably linked to the isolated nucleic acid molecule. In another embodiment the pharmaceutical composition also includes a host cell recombinantly expressing the isolated nucleic acid molecule.

According to another aspect of the invention a pharmaceutical composition is provided. The pharmaceutical composition includes an isolated polypeptide comprising a PP Group 1 or a PP Group 2 polypeptide, and a pharmaceutically acceptable carrier. In one embodiment the isolated polypeptide comprises a PP Group 3 or a PP Group 4 polypeptide.

In another embodiment the isolated polypeptide comprises at least two different polypeptides, each comprising a different cancer associated antigen at least one of which is encoded by a NA group 1 molecule as disclosed herein. In separate embodiments the isolated polypeptides are selected from the following: PP Group 3 polypeptides or HLA binding fragments thereof and PP Group 5 polypeptides or HLA binding fragments thereof.

In an embodiment each of the pharmaceutical compositions described herein also includes an adjuvant.

Another aspect the invention is an isolated nucleic acid molecule comprising a NA Group 3 molecule. Another aspect the invention is an isolated nucleic acid molecule comprising a NA Group 4 molecule.

The invention in another aspect is an isolated nucleic acid molecule selected from the group consisting of (a) a fragment of a nucleic acid selected from the group of nucleic acid molecules consisting of SEQ ID NOs numbered below and comprising all nucleic acid sequences among SEQ ID Nos: 9, 13, 15, 17, 19 and 23, of sufficient length to represent a sequence unique within the mouse or human genomes, and identifying a nucleic acid encoding a cancer associated antigen precursor, (b) complements of (a), provided that the fragment includes a sequence of contiguous nucleotides which is not identical to any sequence selected from the sequence group consisting of (1) sequences having the GenBank accession numbers of Table 11, (2) complements of (1), and (3) fragments of (1) and (2).

In one embodiment the sequence of contiguous nucleotides is selected from the group consisting of: (1) at least two contiguous nucleotides nonidentical to the sequences in Table 11, (2) at least three contiguous nucleotides nonidentical to the sequences in Table 11, (3) at least four contiguous nucleotides nonidentical to the sequences in Table 11, (4) at least five contiguous nucleotides nonidentical to the sequences in Table 11, (5) at least six contiguous nucleotides nonidentical to the sequences in Table 11, or (6) at least seven contiguous nucleotides nonidentical to the sequences in Table 11.

In another embodiment the fragment has a size selected from the group consisting of at least: 8 nucleotides, 10 nucleotides, 12 nucleotides, 14 nucleotides, 16 nucleotides, 18 nucleotides, 20, nucleotides, 22 nucleotides, 24 nucleotides, 26 nucleotides, 28 nucleotides, 30 nucleotides, 50 nucleotides, 75 nucleotides, 100 nucleotides, 200 nucleotides, 1000 nucleotides and every integer length therebetween.

In yet another embodiment the molecule encodes a polypeptide which, or a fragment of which, binds a MHC molecule, preferably a human HLA receptor, or an antibody, preferably one having human amino acid sequences.

Another aspect of the invention is an expression vector comprising an isolated nucleic acid molecule of the invention described above operably linked to a promoter.

According to one aspect the invention is an expression vector comprising a nucleic acid operably linked to a promoter, wherein the nucleic acid is a NA Group 1 or Group 2 molecule. In another aspect the invention is an expression vector comprising a NA Group 1 or Group 2 molecule and a nucleic acid encoding an MHC, preferably HLA, molecule.

In yet another aspect the invention is a host cell transformed or transfected with an expression vector of the invention described above.

In another aspect the invention is a host cell transformed or transfected with an expression vector comprising an isolated nucleic acid molecule of the invention described above operably linked to a promoter, or an expression vector comprising a nucleic acid operably linked to a promoter, wherein the nucleic acid is a NA Group 1 or 2 molecule and further comprising a nucleic acid encoding a MHC molecule.

According to another aspect of the invention an isolated polypeptide encoded by the isolated nucleic acid molecules the invention, described above, is provided. These include PP Group 1-5 polypeptides, including SEQ ID NOS:2, 4, 6, 8, 10, 12, 14, 16, 18, 20; and 24. The invention also includes a fragment of the polypeptide which is immunogenic. In one embodiment the fragment, or a portion of the fragment, binds a MHC molecule, preferably HLA, or an antibody, preferably one having human amino acid sequences.

The invention includes in another aspect an isolated fragment of a cancer associated antigen precursor which, or portion of which, binds a MHC molecule, preferably HLA, or a human antibody, or an antibody, preferably one having human amino acid sequences, wherein the precursor is encoded by a nucleic acid molecule that is a NA Group 1 molecule. In one embodiment the fragment is part of a complex with MHC/HLA. In another embodiment the fragment is between 8 and 12 amino acids in length. In another embodiment the invention includes an isolated polypeptide comprising a fragment of the polypeptide of sufficient length to represent a sequence unique within the mouse or human genomes and identifying a polypeptide that is a cancer associated antigen precursor.

According to another aspect of the invention a kit for detecting the presence of the expression of a cancer associated antigen precursor is provided. The kit includes a pair of isolated nucleic acid molecules each of which consists essentially of a molecule selected from the group consisting of (a) a 12-32 nucleotide contiguous segment of the nucleotide sequence of any of the NA Group 1 molecules and (b) complements of (“a”), wherein the contiguous segments are nonoverlapping. In one embodiment the pair of isolated nucleic acid molecules is constructed and arranged to selectively amplify an isolated nucleic acid molecule that is a NA Group 3 molecule. Preferably, the pair amplifies a human NA Group 3 molecule.

According to another aspect of the invention a method for treating a subject with a disorder characterized by expression of a cancer associated antigen precursor is provided. The method includes the step of administering to the subject an amount of an agent, which enriches selectively in the subject the presence of complexes of a MHC/HLA molecule and a cancer associated antigen, effective to ameliorate the disorder, wherein the cancer associated antigen is a fragment of a cancer associated antigen precursor encoded by a nucleic acid molecule selected from the group consisting of (a) a nucleic acid molecule comprising NA group 1 nucleic acid molecules, (b) a nucleic acid molecule comprising NA group 3 nucleic acid molecules, (c) a nucleic acid molecule comprising NA group 5 nucleic acid molecules.

In one embodiment the disorder is characterized by expression of a plurality of cancer associated antigen precursors and wherein the agent is a plurality of agents, each of which enriches selectively in the subject the presence of complexes of a MHC/HLA molecule and a different cancer associated antigen. Preferably the plurality is at least 2, at least 3, at least 4, or at least 5 such agents.

In another embodiment the agent is an isolated polypeptide selected from the group consisting of PP Group 1, PP Group 2, PP Group 3, PP Group 4, and PP group 5 polypeptides.

In yet another embodiment the disorder is cancer.

According to another aspect the invention is a method for treating a subject having a condition characterized by expression of a cancer associated antigen precursor in cells of the subject. The method includes the steps of (i) removing an immunoreactive cell containing sample from the subject, (ii) contacting the immunoreactive cell containing sample to the host cell under conditions favoring production of cytolytic T cells against a cancer associated antigen which is a fragment of the precursor, (iii) introducing the cytolytic T cells to the subject in an amount effective to lyse cells which express the cancer associated antigen, wherein the host cell is transformed or transfected with an expression vector comprising an isolated nucleic acid molecule operably linked to a promoter, the isolated nucleic acid molecule being selected from the group of nucleic acid molecules consisting of NA Group 1, NA Group 2, NA Group 3, NA Group 4, NA Group 5.

In one embodiment the host cell recombinantly expresses an HLA molecule which binds the cancer associated antigen. In another embodiment the host cell endogenously expresses a MHC/HLA molecule which binds the cancer associated antigen.

The invention includes in another aspect a method for treating a subject having a condition characterized by expression of a cancer associated antigen precursor in cells of the subject. The method includes the steps of (i) identifying a nucleic acid molecule expressed by the cells associated with said condition, wherein said nucleic acid molecule is a NA Group 1 molecule (ii) transfecting a host cell with a nucleic acid selected from the group consisting of (a) the nucleic acid molecule identified, (b) a fragment of the nucleic acid identified which includes a segment coding for a cancer associated antigen, (c) deletions, substitutions or additions to (a) or (b), and (d) degenerates of (a), (b), or (c); (iii) culturing said transfected host cells to express the transfected nucleic acid molecule, and; (iv) introducing an amount of said host cells or an extract thereof to the subject effective to increase an immune response against the cells of the subject associated with the condition. Preferably, the antigen is a human antigen and the subject is a human.

In one embodiment the method also includes the step of (a) identifying a MHC molecule which presents a portion of an expression product of the nucleic acid molecule, wherein the host cell expresses the same MHC molecule as identified in (a) and wherein the host cell presents a MHC binding portion of the expression product of the nucleic acid molecule.

In another embodiment the method also includes the step of treating the host cells to render them non-proliferative.

In yet another embodiment the immune response comprises a B-cell response or a T cell response. Preferably the response is a T-cell response which comprises generation of cytolytic T-cells specific for the host cells presenting the portion of the expression product of the nucleic acid molecule or cells of the subject expressing the cancer associated antigen.

In another embodiment the nucleic acid molecule is a NA Group 3 molecule.

Another aspect of the invention is a method for treating or diagnosing or monitoring a subject having a condition characterized by expression of an abnormal amount of a protein encoded by a nucleic acid molecule that is a NA Group 1 molecule. The method includes the step of administering to the subject an antibody which specifically binds to the protein or a peptide derived therefrom, the antibody being coupled to a therapeutically useful agent, in an amount effective to treat the condition.

In one embodiment the antibody is a monoclonal antibody. Preferably the monoclonal antibody is a chimeric antibody or a humanized antibody.

In another aspect the invention is a method for treating a condition characterized by expression in a subject of abnormal amounts of a protein encoded by a nucleic acid molecule that is a NA Group 1 nucleic acid molecule. The method involves the step of administering to a subject at least one of the pharmaceutical compositions of the invention described above in an amount effective to prevent, delay the onset of, or inhibit the condition in the subject. In one embodiment the condition is cancer. In another embodiment the method includes the step of first identifying that the subject expresses in a tissue abnormal amounts of the protein.

The invention in another aspect is a method for treating a subject having a condition characterized by expression of abnormal amounts of a protein encoded by a nucleic acid molecule that is a NA Group 1 nucleic acid molecule. The method includes the steps of (i) identifying cells from the subject which express abnormal amounts of the protein; (ii) isolating a sample of the cells; (iii) cultivating the cells, and (iv) introducing the cells to the subject in an amount effective to provoke an immune response against the cells.

In one embodiment the method includes the step of rendering the cells non-proliferative, prior to introducing them to the subject.

In another aspect the invention is a method for treating a pathological cell condition characterized by abnormal expression of a protein encoded by a nucleic acid molecule that is a NA Group 1 nucleic acid molecule. The method includes the step of administering to a subject in need thereof an effective amount of an agent which inhibits the expression or activity of the protein.

In one embodiment the agent is an inhibiting antibody which selectively binds to the protein and wherein the antibody is a monoclonal antibody, a chimeric antibody, a humanized antibody or a fragment thereof. In another embodiment the agent is an antisense nucleic acid molecule which selectively binds to the nucleic acid molecule which encodes the protein. In yet another important embodiment the nucleic acid molecule is a NA Group 3 nucleic acid molecule.

The invention includes in another aspect a composition of matter useful in stimulating an immune response to a plurality of proteins encoded by nucleic acid molecules that are NA Group 1 molecules. The composition is a plurality of peptides derived from the amino acid sequences of the proteins, wherein the peptides bind to one or more MHC molecules presented on the surface of the cells which express an abnormal amount of the protein.

In one embodiment at least a portion of the plurality of peptides bind to MHC molecules and elicit a cytolytic response thereto. In another embodiment the composition of matter includes an adjuvant. In another embodiment the adjuvant is a saponin, GM-CSF, or an interleukin. In still another embodiment, the compositions also includes at least one peptide useful in stimulating an immune response to at least one protein which is not encoded by nucleic acid molecules that are NA Group 1 molecules, wherein the at least one peptide binds to one or more MHC molecules.

According to another aspect the invention is an isolated antibody which selectively binds to a complex of: (i) a peptide derived from a protein encoded by a nucleic acid molecule that is a NA Group 1 molecule and (ii) and an MHC molecule to which binds the peptide to form the complex, wherein the isolated antibody does not bind to (i) or (ii) alone.

In one embodiment the antibody is a monoclonal antibody, a chimeric antibody, a humanized antibody or a fragment thereof.

The invention also involves the use of the genes, gene products, fragments thereof, agents which bind thereto, and so on in the preparation of medicaments. A particular medicament is for treating cancer, preferably bladder cancer, colon cancer, lung cancer, breast cancer or hepatoma.

In each of the foregoing embodiments, a preferred nucleic acid molecule include the nucleotide sequence of SEQ ID NO:23 or fragments thereof, and polypeptides comprise SEQ ID NO:24 or fragments thereof, or are encoded by SEQ ID NO:23 or fragments thereof.

In a further aspect of the invention, nucleic acid microarrays that include a NA Group 1 through 5 nucleic acid molecule are provided. In preferred embodiments, the nucleic acid molecule comprises SEQ ID NO:23, or a fragment thereof. Protein microarrays that include a PP Group 1 through 5 polypeptide also are provided. In preferred embodiments, the polypeptide comprises SEQ ID NO:24, or a fragment thereof, preferably an immunogenic fragment. The use of such microarrays in diagnostic applications, particularly for diagnosing cancer, also is provided.

These and other aspects of the invention will be described in further detail in connection with the detailed description of the invention.

### BRIEF DESCRIPTION OF THE DRAWINGS

FIG. 1 shows an EST cluster for OY-TES-1 cDNA. Each arrow represents an EST sequence in the cluster.

FIG. 2 shows nucleotide (SEQ ID NO: 23) and deduced amino acid (SEQ ID NO: 24) sequences of OY-TES-1. Primers used for PCR are indicated by arrows. Polyadenylation signal consensus sequence is underlined. The sequence has been deposited in the GenBank under accession number ______.

FIG. 3 shows sequence alignment of guinea pig (GP; SEQ ID NO:37), porcine (Pig; SEQ ID NO: 38), mouse (SEQ ID NO: 39), and human (OY-TES-1, SEQ ID NO: 23) sp32 precursor protein. The conserved residues are indicated by asterisks. The dots indicate the identical residues with those in the guinea pig. Twenty cysteine residues are totally conserved (highlighted in black). A glutamic acid- and glutamine rich-domain (EQ-rich domain) is boxed. An acidic amino acid region is underlined. Identity: guinea pig/human, 77.2%; porcine/human, 81.9%; mouse/human, 75.2%.

FIG. 4 shows Southern blot analysis of the OY-TES-1 gene. Genomic DNA from normal testis was digested with EcoRI, HindIII, and BamHI and analyzed with an OY-TES-1 probe. EcoRI and BamHI digests showed a strong and a weak band, suggesting that there are two homologue genes in the human genome.

FIG. 5 is a photomicrograph showing chromosome localization of OY-TES-1 gene by fluorescence in situ hybridization. Metaphases showed twin signals with brightly red fluorescence on 12p12-p13 (arrows). The chromosome 12-specific a satellite probe at the centromere was also shown in green fluorescence.

FIG. 6 is a diagram shows genomic structure of OY-TES-1. The open reading frame is shown in shaded boxes and spliced introns as lines. The exon/intron stricture is determined based on the PAC clone RP4-761J14 and BAC clone RP11-433J6.

FIG. 7 shows RT-PCR analysis of OY-TES-1 expression in normal tissues. PCR primers were: ht-5,5′-AAGGACAGGGGACTAAGGAG-3′ (SEQ ID NO: 27) and ht-3,5′-CCGTACAAATCCAGCCCGTA-3′ (SEQ ID NO: 28). The same cDNA samples were tested for β-actin as an internal control. OY-TES-1 restriction was restricted to testis.

FIG. 8 shows RT-PCR analysis of OY-TES-1 expression in tumors using primers ht-5 and ht-3; The same cDNA samples were tested for β-actin as an internal control.

FIG. 9 shows representative results of ELISA reactivity with sera from bladder and prostate cancer patients.

### DETAILED DESCRIPTION OF THE INVENTION

In the above summary and in the ensuing description, lists of sequences are provided. The lists are meant to embrace each single sequence separately, two or more sequences together where they form a part of the same gene, any combination of two or more sequences which relate to different genes, including and up to the total number on the list, as if each and every combination were separately and specifically enumerated. Likewise, when mentioning fragment size, it is intended that a range embrace the smallest fragment mentioned to the full-length of the sequence (less one nucleotide or amino acid so that it is a fragment), each and every fragment length intended as if specifically enumerated. Thus, if a fragment could be between 10 and 15 in length, it is explicitly meant to mean 10, 11, 12, 13, 14, or 15 in length.

The summary and the claims mention antigen precursors and antigens. As used in the summary and in the claims, a precursor is substantially the full-length protein encoded by the coding region of the isolated DNA and the antigen is a peptide which complexes with MHC, preferably HLA, and which participates in the immune response as part of that complex. Such antigens are typically 9 amino acids long, although this may vary slightly.

As used herein, a subject is a human, non-human primate, cow, horse, pig, sheep, goat, dog, cat or rodent. In all embodiments human cancer associated antigens, which have substantial nucleotide and/or amino acid sequence identity to the presently identified cancer associated antigens, and human subjects are preferred.

The present invention in one aspect involves the cloning of cDNAs encoding cancer associated antigen precursors using autologous antisera of mice having methylcholanthrene-induced fibrosarcomas. The sequences of the clones representing genes identified according to the methods described herein are presented in the attached Sequence Listing. Of the foregoing, it can be seen that some of the clones are considered completely novel as no nucleotide or amino acid homologies to coding regions were found in the databases searched. Other clones are novel but have some homology to sequences deposited in databases (mainly EST sequences). Nevertheless, the entire gene sequence was not previously known. In some cases no function was suspected and in other cases, even if a function was suspected, it was not know that the gene was associated with cancer. In all cases, it was not known or suspected that the gene encoded a cancer antigen which reacted with antibody from autologous sera. Analysis of the clone sequences by comparison to nucleic acid and protein databases determined that still other of the clones surprisingly are closely related to other previously-cloned genes. The sequences of these related genes is also presented in the Sequence Listing. The nature of the foregoing genes as encoding antigens recognized by the immune systems of cancer patients is, of course, unexpected.

The invention thus involves in one aspect cancer associated antigen polypeptides, genes encoding those polypeptides, functional modifications and variants of the foregoing, useful fragments of the foregoing, as well as diagnostics and therapeutics relating thereto.

Homologs and alleles of the cancer associated antigen nucleic acids of the invention can be identified by conventional techniques. Thus, an aspect of the invention is those nucleic acid sequences which code for cancer associated antigen precursors. The following chart is provided to identify the various groups of sequences discussed in the claims and in the summary:

### Nucleic Acid Sequences

NA Group 1

(a) nucleic acid molecules which hybridize under stringent conditions to a molecule consisting of a nucleic acid sequence selected from the group consisting of nucleic acid sequences among SEQ ID NOs: 1, 3, 5, 7, 9, 11, 13, 15, 17, 19 and 23 and which code for a cancer associated antigen precursor,

(b) deletions, additions and substitutions which code for a respective cancer associated antigen precursor,

(c) nucleic acid molecules that differ from the nucleic acid molecules of (a) or

(b) in codon sequence due to the degeneracy of the genetic code, and

(d) complements of (a), (b) or (c).

NA Group 2. Fragments of NA Group 1, which codes for a polypeptide which, or a portion of which, binds an MHC molecule to form a complex recognized by a an autologous antibody or lymphocyte.

NA Group 3. The subset of NA Group 1 where the nucleotide sequence is selected from the group consisting of:

(a) previously unknown nucleic acids coding for a cancer associated antigen precursor,

(b) deletions, additions and substitutions which code for a respective cancer associated antigen precursor,

(c) nucleic acid molecules that differ from the nucleic acid molecules of (a) or

(b) in codon sequence due to the degeneracy of the genetic code, and

(d) complements of (a), (b) or (c).

NA Group 4. Fragments of NA Group 3, which code for a polypeptide which, or a portion of which, binds to an MHC molecule to form a complex recognized by an autologous antibody or lymphocyte.

NA Group 5. A subset of NA Group 1, comprising cancer associated antigens that react with allogeneic cancer antisera.

### Polypeptide Sequences

PP Group 1. Polypeptides encoded by NA Group 1. PP Group 2. Polypeptides encoded by NA Group 2

PP Group 3. Polypeptides encoded by NA Group 3.

PP Group 4. Polypeptides encoded by NA Group 4.

PP Group 5. Polypeptides encoded by NA Group 5.

Identification of human homologs of cancer associated antigens will be familiar to those of skill in the art. In particular, the methods described in Example 8 were effective in identifying the human homolog of OY-MC4, OY-TES-1, as well as establishing it as a cancer-testis antigen useful in therapeutic and diagnostic applications for treatment and diagnosis of cancer.

In general, nucleic acid hybridization is a suitable method for identification of homologous sequences of another species (e.g., human) which correspond to a known sequence (e.g., mouse cancer associated sequences presented herein). Standard nucleic acid hybridization procedures can be used to identify related nucleic acid sequences of selected precent identity. For example, one can construct a library of cDNAs reverse transcribed from the mRNA of a selected tissue (e.g., testis) and use the cancer associated antigen nucleic acids identified herein to screen the library for related nucleotide sequences. The screening can be performed at various stringencies to identify those sequences which are closely related by sequence identity, and more distantly related. Nucleic acids so identified ca be translated into polypeptides and the polypeptides can be tested for activity.

Identification of related sequences can also be achieved using polymerase chain reaction (PCR) and other amplification techniques suitable for cloning related nucleic acid sequences. Preferably PCR primers are selected to amplify portions of a nucleic acid sequence believed to be conserved (e.g., a catalytic domain, a DNA-binding domain, etc.). Again, nucleic acids are preferably amplified from a tissue-specific library (e.g., such as testis to find sequences related to OY-MC4). One also can use expression cloning utilizing the antisera described herein to identify nucleic acids which encode related antigenic proteins in humans or other species using the SEREX procedure.

The term “stringent conditions” as used herein refers to parameters with which the art is familiar. Nucleic acid hybridization parameters may be found in references which compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. More specifically, stringent conditions, as used herein, refers, for example, to hybridization at 65° C. in hybridization buffer (3.5×SSC, 0.02% Ficoll, 0.02% polyvinyl pyrrolidone, 0.02% Bovine Serum Albumin, 2.5 mM NaH₂PO₄(pH 7), 0.5% SDS, 2 mM EDTA). SSC is 0.15M sodium chloride/0.015M sodium citrate, pH 7; SDS is sodium dodecyl sulphate; and EDTA is ethylenediaminetetracetic acid. After hybridization, the membrane upon which the DNA is transferred is washed, for example, in 2×SSC at room temperature and then at 0.1-0.5×SSC/0.1×SDS at temperatures up to 68° C.

There are other conditions, reagents, and so forth which can be used, which result in a similar degree of stringency. The skilled artisan will be familiar with such conditions, and thus they are not given here. It will be understood, however, that the skilled artisan will be able to manipulate the conditions in a manner to permit the clear identification of homologs and alleles of cancer associated antigen nucleic acids of the invention (e.g., by using lower stringency conditions). The skilled artisan also is familiar with the methodology for screening cells and libraries for expression of such molecules which then are routinely isolated, followed by isolation of the pertinent nucleic acid molecule and sequencing.

In general homologs and alleles typically will share at least 75% nucleotide identity and/or at least 90% amino acid identity to the sequences of cancer associated antigen nucleic acid and polypeptides, respectively, in some instances will share at least 90% nucleotide identity and/or at least 95% amino acid identity and in still other instances will share at least 95% nucleotide identity and/or at least 99% amino acid identity. The homology can be calculated using various, publicly available software tools developed by NCBI (Bethesda, Md.) that can be obtained through the internet (ftp:/ncbi.nlm.nih.gov/pub/). Exemplary tools include the BLAST system available at http://www.ncbi.nlm.nih.gov, using default settings. Pairwise and ClustalW alignments (BLOSUM30 matrix setting) as well as Kyte-Doolittle hydropathic analysis can be obtained using the MacVector sequence analysis software (Oxford Molecular Group). Watson-Crick complements of the foregoing nucleic acids also are embraced by the invention.

In screening for cancer associated antigen genes, a Southern blot may be performed using the foregoing conditions, together with a radioactive probe. After washing the membrane to which the DNA is finally transferred, the membrane can be placed against X-ray film to detect the radioactive signal. In screening for the expression of cancer associated antigen nucleic acids, Northern blot hybridizations using the foregoing conditions (see also the Examples) can be performed on samples taken from cancer patients or subjects suspected of having a condition characterized by expression of cancer associated antigen genes. Amplification protocols such as polymerase chain reaction using primers which hybridize to the sequences presented also can be used for detection of the cancer associated antigen genes or expression thereof.

As used herein with respect to nucleic acids, the term “isolated” means: (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid is one which is readily manipulable by recombinant DNA techniques well known in the art. Thus, a nucleotide sequence contained in a vector in which 5′ and 3′ restriction sites are known or for which polymerase chain reaction (PCR) primer sequences have been disclosed is considered isolated but a nucleic acid sequence existing in its native state in its natural host is not. An isolated nucleic acid may be substantially purified, but need not be. For example, a nucleic acid that is isolated within a cloning or expression vector is not pure in that it may comprise only a tiny percentage of the material in the cell in which it resides. Such a nucleic acid is isolated, however, as the term is used herein because it is readily manipulable by standard techniques known to those of ordinary skill in the art. An isolated nucleic acid as used herein is not a naturally occurring chromosome.

As used herein with respect to polypeptides, “isolated” means separated from its native environment and present in sufficient quantity to permit its identification or use. Isolated, when referring to a protein or polypeptide, means, for example: (i) selectively produced by expression cloning or (ii) purified as by chromatography or electrophoresis. Isolated proteins or polypeptides may, but need not be, substantially pure. The term “substantially pure” means that the proteins or polypeptides are essentially free of other substances with which they may be found in nature or in vivo systems to an extent practical and appropriate for their intended use. Substantially pure polypeptides may be produced by techniques well known in the art. Because an isolated protein may be admixed with a pharmaceutically acceptable carrier in a pharmaceutical preparation, the protein may comprise only a small percentage by weight of the preparation. The protein is nonetheless isolated in that it has been separated from the substances with which it may be associated in living systems, i.e. isolated from other proteins.

The cancer associated genes correspond to SEQ ID Nos: 1, 3, 5, 7, 9, 11, 13, 15, 17, 19 and 23. The preferred cancer associated antigens for the methods of diagnosis disclosed herein are those which are found to react with allogeneic cancer antisera (i.e. NA Group 5). Encoded polypeptides (e.g., proteins), peptides and antisera thereto are also preferred for diagnosis. Most preferably, the nucleic acid is SEQ ID NO:23 and the polypeptide is SEQ ID NO:24.

The invention also includes degenerate nucleic acids which include alternative codons to those present in the native materials. For example, serine residues are encoded by the codons TCA, AGT, TCC, TCG, TCT and AGC. Each of the six codons is equivalent for the purposes of encoding a serine residue. Thus, it will be apparent to one of ordinary skill in the art that any of the serine-encoding nucleotide triplets may be employed to direct the protein synthesis apparatus, in vitro or in vivo, to incorporate a serine residue into an elongating cancer associated antigen polypeptide. Similarly, nucleotide sequence triplets which encode other amino acid residues include, but are not limited to: CCA, CCC, CCG and CCT (proline codons); CGA, CGC, CGG, CGT, AGA and AGG (arginine codons); ACA, ACC, ACG and ACT (threonine codons); AAC and AAT (asparagine codons); and ATA, ATC and ATT (isoleucine codons). Other amino acid residues may be encoded similarly by multiple nucleotide sequences. Thus, the invention embraces degenerate nucleic acids that differ from the biologically isolated nucleic acids in codon sequence due to the degeneracy of the genetic code.

The invention also provides modified nucleic acid molecules which include additions, substitutions and deletions of one or more nucleotides. In preferred embodiments, these modified nucleic acid molecules and/or the polypeptides they encode retain at least one activity or function of the unmodified nucleic acid molecule and/or the polypeptides, such as antigenicity, enzymatic activity, receptor binding, formation of complexes by binding of peptides by MHC class I and class II molecules, etc. In certain embodiments, the modified nucleic acid molecules encode modified polypeptides, preferably polypeptides having conservative amino acid substitutions as are described elsewhere herein. The modified nucleic acid molecules are structurally related to the unmodified nucleic acid molecules and in preferred embodiments are sufficiently structurally related to the unmodified nucleic acid molecules so that the modified and unmodified nucleic acid molecules hybridize under stringent conditions known to one of skill in the art.

For example, modified nucleic acid molecules which encode polypeptides having single amino acid changes can be prepared. Each of these nucleic acid molecules can have one, two or three nucleotide substitutions exclusive of nucleotide changes corresponding to the degeneracy of the genetic code as described herein. Likewise, modified nucleic acid molecules which encode polypeptides having two amino acid changes can be prepared which have, e.g., 2-6 nucleotide changes. Numerous modified nucleic acid molecules like these will be readily envisioned by one of skill in the art, including for example, substitutions of nucleotides in codons encoding amino acids 2 and 3, 2 and 4, 2 and 5, 2 and 6, and so on. In the foregoing example, each combination of two amino acids is included in the set of modified nucleic acid molecules, as well as all nucleotide substitutions which code for the amino acid substitutions. Additional nucleic acid molecules that encode polypeptides having additional substitutions (i.e., 3 or more), additions or deletions (e.g., by introduction of a stop codon or a splice site(s)) also can be prepared and are embraced by the invention as readily envisioned by one of ordinary skill in the art. Any of the foregoing nucleic acids or polypeptides can be tested by routine experimentation for retention of structural relation or activity to the nucleic acids and/or polypeptides disclosed herein.

The invention also provides isolated fragments of cancer associated antigen nucleic acid sequences or complements thereof, preferably unique fragments. A unique fragment is one that is a ‘signature’ for the larger nucleic acid. It, for example, is long enough to assure that its precise sequence is not found in molecules within the human genome outside of the cancer associated antigen nucleic acids defined above (and human alleles). Those of ordinary skill in the art may apply no more than routine procedures to determine if a fragment is unique within the human genome. Unique fragments, however, exclude fragments completely composed of the nucleotide sequences of any of GenBank accession numbers listed in Table 11 or other previously published sequences as of the filing date of the priority documents for sequences listed in a respective priority document or the filing date of this application for sequences listed for the first time in this application which overlap the sequences of the invention.

A fragment which is completely composed of the sequence described in the foregoing GenBank deposits is one which does not include any of the nucleotides unique to the sequences of the invention. Thus, a unique fragment must contain a nucleotide sequence other than the exact sequence of those in GenBank or fragments thereof. The difference may be an addition, deletion or substitution with respect to the GenBank sequence or it may be a sequence wholly separate from the GenBank sequence.

Fragments, preferably unique fragments, can be used as probes in Southern and Northern blot assays to identify such nucleic acids, or can be used in amplification assays such as those employing PCR. As known to those skilled in the art, large probes such as 200, 250, 300 or more nucleotides are preferred for certain uses such as Southern and Northern blots, while smaller fragments will be preferred for uses such as PCR. Fragments also can be used to produce fusion proteins for generating antibodies or determining binding of the polypeptide fragments, or for generating immunoassay components. Likewise, fragments can be employed to produce nonfused fragments of the cancer associated antigen polypeptides, useful, for example, in the preparation of antibodies, and in immunoassays. Fragments further can be used as antisense molecules to inhibit the expression of cancer associated antigen nucleic acids and polypeptides, particularly for therapeutic purposes as described in greater detail below.

As will be recognized by those skilled in the art, the size of the unique fragment will depend upon its conservancy in the genetic code. Thus, some regions of cancer associated antigen sequences and complements thereof will require longer segments to be unique while others will require only short segments, typically between 12 and 32 nucleotides (e.g. 12, 13, 14, 15, 16, 17, 18, 19, 20, 21, 22, 23, 24, 25, 26, 27, 28, 29, 30, 31 and 32 or more bases long, up to the entire length of the disclosed sequence. As mentioned above, this disclosure intends to embrace each and every fragment of each sequence, beginning at the first nucleotide, the second nucleotide and so on, up to 8 nucleotides short of the end, and ending anywhere from nucleotide number 8, 9, 10 and so on for each sequence, up to the very last nucleotide (provided the sequence is unique as described above).

Many segments of the polypeptide coding region of novel cancer associated antigen nucleic acids, or complements thereof, that is 18 or more nucleotides in length will be unique. Those skilled in the art are well versed in methods for selecting such sequences, typically on the basis of the ability of the unique fragment to selectively distinguish the sequence of interest from other sequences in the human genome of the fragment to those on known databases typically is all that is necessary, although in vitro confirmatory hybridization and sequencing analysis may be performed.

Especially preferred include nucleic acids encoding a series of epitopes, known as “polytopes”. The epitopes can be arranged in sequential or overlapping fashion (see, e.g., Thomson et al., Proc. Natl Acad. Sci. USA 92:5845-5849, 1995; Gilbert et al., Nature Biotechnol. 15:1280-1284, 1997), with or without the natural flanking sequences, and can be separated by unrelated linker sequences if desired. The polytope is processed to generated individual epitopes which are recognized by the immune system for generation of immune responses.

Thus, for example, peptides derived from a polypeptide having an amino acid sequence encoded by one of the nucleic acid disclosed herein, and which are presented by MHC molecules and recognized by CTL or T helper lymphocytes, can be combined with peptides from one or more other cancer associated antigens (e.g. by preparation of hybrid nucleic acids or polypeptides) to form “polytopes”. The two or more peptides (or nucleic acids encoding the peptides) can be selected from those described herein, or they can include one or more peptides of previously known cancer associated antigens. Exemplary cancer associated peptide antigens that can be administered to induce or enhance an immune response are derived from tumor associated genes and encoded proteins including MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-A13, GAGE-1, GAGE-2, GAGE-3, GAGE-4, GAGE-5, GAGE-6, GAGE-7, GAGE-8, BAGE-1, RAGE-1, LB33/MUM-1, PRAME, NAG, MAGE-B2, MAGE-B3, MAGE-B4, tyrosinase, brain glycogen phosphorylase, Melan-A, MAGE-C1, MAGE-C2, NY-ESO-1, LAGE-1, SSX-1, SSX-2 (HOM-MEL40), SSX4, SSX-5, SCP-1 and CT-7. See, for example, PCT application publication no. WO96/10577. Other examples will be known to one of ordinary skill in the art (for example, see Coulie, Stem Cells 13:393-403, 1995), and can be used in the invention in a like manner as those disclosed herein. One of ordinary skill in the art can prepare polypeptides comprising one or more peptides and one or more of the foregoing cancer associated peptides, or nucleic acids encoding such polypeptides, according to standard procedures of molecular biology.

Thus polytopes are groups of two or more potentially immunogenic or immune response stimulating peptides which can be joined together in various arrangements (e.g. concatenated, overlapping). The polytope (or nucleic acid encoding the polytope) can be administered in a standard immunization protocol, e.g. to animals, to test the effectiveness of the polytope in stimulating, enhancing and/or provoking an immune response.

The peptides can be joined together directly or via the use of flanking sequences to form polytopes, and the use of polytopes as vaccines is well known in the art (see, e.g., Thomson et al., Proc. Acad. Natl. Acad Sci USA 92(13):5845-5849, 1995; Gilbert et al., Nature Biotechnol. 15(12):1280-1284, 1997; Thomson et al., J. Immunol. 157(2):822-826, 1996; Tam et al., J. Exp. Med. 171(1):299-306, 1990). For example, Tam showed that polytopes consisting of both MHC class I and class II binding epitopes successfully generated antibody and protective immunity in a mouse model. Tam also demonstrated that polytopes comprising “strings” of epitopes are processed to yield individual epitopes which are presented by MHC molecules and recognized by CTLs. Thus polytopes containing various numbers and combinations of epitopes can be prepared and tested for recognition by CTLs and for efficacy in increasing an immune response.

It is known that tumors express a set of tumor antigens, of which only certain subsets may be expressed in the tumor of any given patient. Polytopes can be prepared which correspond to the different combination of epitopes representing the subset of tumor rejection antigens expressed in a particular patient. Polytopes also can be prepared to reflect a broader spectrum of tumor rejection antigens known to be expressed by a tumor type. Polytopes can be introduced to a patient in need of such treatment as polypeptide structures, or via the use of nucleic acid delivery systems known in the art (see, e.g., Allsopp et al., Eur. J. Immunol. 26(8):1951-1959, 1996). Adenovirus, pox virus, Ty-virus like particles, adeno-associated virus, plasmids, bacteria, etc. can be used in such delivery. One can test the polytope delivery systems in mouse models to determine efficacy of the delivery system. The systems also can be tested in human clinical trials.

In instances in which a MHC class I molecule presents tumor rejection antigens derived from cancer associated nucleic acids, the expression vector may also include a nucleic acid sequence coding for the MHC molecule that presents any particular tumor rejection antigen derived from these nucleic acids and polypeptides. Alternatively, the nucleic acid sequence coding for such a MHC molecule can be contained within a separate expression vector. In a situation where the vector contains both coding sequences, the single vector can be used to transfect a cell which does not normally express either one. Where the coding sequences for a cancer associated antigen precursor and the MHC molecule which presents it are contained on separate expression vectors, the expression vectors can be cotransfected. The cancer associated antigen precursor coding sequence may be used alone, when, e.g. the host cell already expresses a MHC molecule which presents a cancer associated antigen derived from precursor molecules. Of course, there is no limit on the particular host cell which can be used. As the vectors which contain the two coding sequences may be used in any antigen-presenting cells if desired, and the gene for cancer associated antigen precursor can be used in host cells which do not express a MHC molecule which presents a cancer associated antigen. Further, cell-free transcription systems may be used in lieu of cells.

As mentioned above, the invention embraces antisense oligonucleotides that selectively bind to a nucleic acid molecule encoding a cancer associated antigen polypeptide, to reduce the expression of cancer associated antigens. This is desirable in virtually any medical condition wherein a reduction of expression of cancer associated antigens is desirable, e.g., in the treatment of cancer. This is also useful for in vitro or in vivo testing of the effects of a reduction of expression of one or more cancer associated antigens.

As used herein, the term “antisense oligonucleotide” or “antisense” describes an oligonucleotide that is an oligoribonucleotide, oligodeoxyribonucleotide, modified oligoribonucleotide, or modified oligodeoxyribonucleotide which hybridizes under physiological conditions to DNA comprising a particular gene or to an mRNA transcript of that gene and, thereby, inhibits the transcription of that gene and/or the translation of that mRNA. The antisense molecules are designed so as to interfere with transcription or translation of a target gene upon hybridization with the target gene or transcript. Those skilled in the art will recognize that the exact length of the antisense oligonucleotide and its degree of complementarity with its target will depend upon the specific target selected, including the sequence of the target and the particular bases which comprise that sequence. It is preferred that the antisense oligonucleotide be constructed and arranged so as to bind selectively with the target under physiological conditions, i.e., to hybridize substantially more to the target sequence than to any other sequence in the target cell under physiological conditions. Based upon the sequences of nucleic acids encoding cancer associated antigens, or upon allelic or homologous genomic and/or cDNA sequences, one of skill in the art can easily choose and synthesize any of a number of appropriate antisense molecules for use in accordance with the present invention. In order to be sufficiently selective and potent for inhibition, such antisense oligonucleotides should comprise at least 10 and, more preferably, at least 15 consecutive bases which are complementary to the target, although in certain cases modified oligonucleotides as short as 7 bases in length have been used successfully as antisense oligonucleotides (Wagner et al., Nature Biotechnol. 14:840-844, 1996). Most preferably, the antisense oligonucleotides comprise a complementary sequence of 20-30 bases. Although oligonucleotides may be chosen which are antisense to any region of the gene or mRNA transcripts, in preferred embodiments the antisense oligonucleotides correspond to N-terminal or 5′ upstream sites such as translation initiation, transcription initiation or promoter sites. In addition, 3′-untranslated regions may be targeted. Targeting to mRNA splicing sites has also been used in the art but may be less preferred if alternative mRNA splicing occurs. In addition, the antisense is targeted, preferably, to sites in which mRNA secondary structure is not expected (see, e.g., Sainio et al., Cell Mol. Neurobiol. 14(5):439457, 1994) and at which proteins are not expected to bind. Finally, although the listed sequences are cDNA sequences, one of ordinary skill in the art may easily derive the genomic DNA corresponding to the cDNA of a cancer associated antigen. Thus, the present invention also provides for antisense oligonucleotides which are complementary to the genomic DNA corresponding to nucleic acids encoding cancer associated antigens. Similarly, antisense to allelic cDNAs, homologous cDNAs (e.g., human) and genomic DNAs are enabled without undue experimentation.

In one set of embodiments, the antisense oligonucleotides of the invention may be composed of “natural” deoxyribonucleotides, ribonucleotides, or any combination thereof. That is, the 5′ end of one native nucleotide and the 3′ end of another native nucleotide may be covalently linked, as in natural systems, via a phosphodiester internucleoside linkage. These oligonucleotides may be prepared by art recognized methods which may be carried out manually or by an automated synthesizer. They also may be produced recombinantly by vectors.

In preferred embodiments, however, the antisense oligonucleotides of the invention also may include “modified” oligonucleotides. That is, the oligonucleotides may be modified in a number of ways which do not prevent them from hybridizing to their target but which enhance their stability or targeting or which otherwise enhance their therapeutic effectiveness.

The term “modified oligonucleotide” as used herein describes an oligonucleotide in which (1) at least two of its nucleotides are covalently linked via a synthetic internucleoside linkage (i.e., a linkage other than a phosphodiester linkage between the 5′ end of one nucleotide and the 3′ end of another nucleotide) and/or (2) a chemical group not normally associated with nucleic acids-has been-covalently attached to the oligonucleotide. Preferred synthetic internucleoside linkages are phosphorothioates, alkylphosphonates, phosphorodithioates, phosphate esters, alkylphosphonothioates, phosphoramidates, carbarnates, carbonates, phosphate triesters, acetamidates, carboxymethyl esters and peptides.

The term “modified oligonucleotide” also encompasses oligonucleotides with a covalently modified base and/or sugar. For example, modified oligonucleotides include oligonucleotides having backbone sugars which are covalently attached to low molecular weight organic groups other than a hydroxyl group at the 3′ position and other than a phosphate group at the 5′ position. Thus modified oligonucleotides may include a 2′-O-alkylated ribose group. In addition, modified-oligonucleotides may include sugars such as arabinose instead of ribose. The present invention, thus, contemplates pharmaceutical preparations containing modified antisense molecules that are complementary to and hybridizable with, under physiological conditions, nucleic acids encoding cancer associated antigen polypeptides, together with pharmaceutically acceptable carriers.

As used herein, a “vector” may be any of a number of nucleic acids into which a desired sequence may be inserted by restriction and ligation for transport between different genetic environments or for expression in a host cell. Vectors are typically composed of DNA although RNA vectors are also available. Vectors include, but are not limited to, plasmids, phagemids and virus genomes. A cloning vector is one which is able to replicate autonomously or integrated in the genone in a host cell, and which is further characterized by one or more endonuclease restriction sites at which the vector may be cut in a determinable fashion and into which a desired DNA sequence may be ligated such that the new recombinant vector retains its ability to replicate in the host cell. In the case of plasmids, replication of the desired sequence may occur many times as the plasmid increases in copy number within the host bacterium or just a single time per host before the host reproduces by mitosis. In the case of phage, replication may occur actively during a lytic phase or passively during a lysogenic phase. An expression vector is one into which a desired DNA sequence may be inserted by restriction and ligation such that it is operably joined to regulatory sequences and may be expressed as an RNA transcript. Vectors may further contain one or more marker sequences suitable for use in the identification of cells which have or have not been transformed or transfected with the vector. Markers include, for example, genes encoding proteins which increase or decrease either resistance or sensitivity to antibiotics or other compounds, genes which encode enzymes whose activities are detectable by standard assays known in the art (e.g., β-galactosidase, luciferase or alkaline phosphatase), and genes which visibly affect the phenotype of transformed or transfected cells, hosts, colonies or plaques (e.g., green fluorescent protein). Preferred vectors are those capable of autonomous replication and expression of the structural gene products present in the DNA segments to which they are operably joined.

As used herein, a coding sequence and regulatory sequences are said to be “operably” joined when they are covalently linked in such a way as to place the expression or transcription of the coding sequence under the influence or control of the regulatory sequences. If it is desired that the coding sequences be translated into a functional protein, two DNA sequences are said to be operably joined if-induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably joined to a coding sequence if the promoter region were capable of effecting transcription of that DNA sequence such that the resulting transcript might be translated into the desired protein or polypeptide.

The precise nature of the regulatory sequences needed for gene expression may vary between species or cell types, but shall in general include, as necessary, 5′ non-transcribed and 5′ non-translated sequences involved with the initiation of transcription and translation respectively, such as a TATA box, capping sequence, CAAT sequence, and the like. Especially, such 5′ non-transcribed regulatory sequences will include a promoter region which includes a promoter sequence for transcriptional control of the operably joined gene. Regulatory sequences may also include enhancer sequences or upstream activator sequences as desired. The vectors of the invention may optionally include 5′ leader or signal sequences. The choice and design of an appropriate vector is within the ability and discretion of one of ordinary skill in the art.

Expression vectors containing all the necessary elements for expression are commercially available and known to those skilled in the art. See, e.g., Sambrook et al., Molecular Cloning: A Laboratory Manual, Second Edition, Cold Spring Harbor Laboratory Press, 1989. Cells are genetically engineered by the introduction into the cells of heterologous DNA (RNA) encoding a cancer associated antigen polypeptide or fragment or variant thereof. That heterologous DNA (RNA) is placed under operable control of transcriptional elements to permit the expression of the heterologous DNA in the host cell.

Preferred systems for mRNA expression in mammalian cells are those such as pcDNA3.1 or pRc/CMV (available from Invitrogen, Carlsbad, Calif.) that contain a selectable marker such as a gene that confers G418 resistance (which facilitates the selection of stably transfected cell lines) and the human cytomegalovirus (CMV) enhancer-promoter sequences. Additionally, suitable for expression in primate or canine cell lines is the pCEP4 vector (Invitrogen), which contains an Epstein Barr Virus (EBV) origin of replication, facilitating the maintenance of plasmid as a multicopy extrachromosomal element. Another expression vector is the pEF-BOS plasmid containing the promoter of polypeptide Elongation Factor 1α, which stimulates efficiently transcription in vitro. The plasmid is described by Mishizuma and Nagata (Nuc. Acids Res. 18:5322, 1990), and its use in transfection experiments is disclosed by, for example, Demoulin (Mol. Cell. Biol. 16:47104716, 1996). Still another preferred expression vector is an adenovirus, described by Stratford-Perricaudet, which is defective for E1 and E3 proteins (J. Clin. Invest. 90:626-630, 1992). The use of the adenovirus as an Adeno.P1A recombinant for the expression of an antigen is disclosed by Warnier et al., in intradermal injection in mice for immunization against P1A (Int. J. Cancer, 67:303-310, 1996). Additional vectors for delivery of nucleic acid are provided below.

The invention also embraces so-called expression kits, which allow the artisan to prepare a desired expression vector or vectors. Such expression kits include at least separate portions of a vector and one or more of the previously discussed cancer associated antigen nucleic acid molecules. Other components may be added, as desired, as long as the previously mentioned nucleic acid molecules, which are required, are included. The invention also includes kits for amplification of a cancer associated antigen nucleic acid, including at least one pair of amplification primers which hybridize to a cancer associated antigen nucleic acid. The primers preferably are 12-32 nucleotides in length and are non-overlapping to prevent formation of “primer-dimers”. One of the primers will hybridize to one strand of the cancer associated antigen nucleic acid and the second primer will hybridize to the complementary strand of the cancer associated antigen nucleic acid, in an arrangement which permits amplification of the cancer associated antigen nucleic acid. Selection of appropriate primer pairs is standard in the art. For example, the selection can be made with assistance of a computer program designed for such a purpose, optionally followed by testing the primers for amplification specificity and efficiency.

The invention further includes nucleic acid or protein microarrays which include nucleic acid molecules selected from NA Groups 1 through 5 or polypeptides selected from PP Groups 1 through 5. Preferably the microarrays contain SEQ ID NO:23 or SEQ ID NO:24, respectively.

In one aspect, standard hybridization techniques of microarray technology are utilized to assess patterns of nucleic acid expression and identify nucleic acid marker expression. Nucleic acid microarray technology, which is also known by other names including: DNA chip technology, gene chip technology, and solid-phase nucleic acid array technology, is well known to those of ordinary skill in the art and is based on, but not limited to, obtaining an array of identified nucleic acid probes on a fixed substrate, labeling target molecules with reporter molecules (e.g., radioactive, chemiluminescent, or fluorescent tags such as fluorescein, Cye3-dUTP, or Cye5-dUTP), hybridizing target nucleic acids to the probes, and evaluating target-probe hybridization. A probe with a nucleic acid sequence that perfectly matches the target sequence will, in general, result in detection of a stronger reporter-molecule signal than will probes with less perfect matches. Many components and techniques utilized in nucleic acid microarray technology are presented in “The Chipping Forecast”, Nature Genetics, Vol.21, January 1999, the entire contents of which is incorporated by reference herein.

In another aspect, standard techniques of protein microarray technology are utilized to assess expression of the polypeptides of the invention, and fragments thereof (including HLA binding peptides, and/or identify biological constituents that bind such proteins and peptides. Protein microarray technology, which is also known by other names including: protein chip technology and solid-phase protein array technology, is well known to those of ordinary skill in the art and is based on, but not limited to, obtaining an array of identified peptides or proteins on a fixed substrate, binding target molecules or biological constituents to the peptides, and evaluating such binding. See, e.g., G. MacBeath and S. L. Schreiber, “Printing Proteins as Microarrays for High-Throughput Function Determination,” Science 289(5485):1760-1763, 2000. The constituents of biological samples that can be analyzed by protein microarrays include polypeptides, antibodies, HLA molecules, lymphocytes (particularly T lymphocytes), and the like.

According to the present invention, microarray substrates may include but are not limited to glass, silica, aluminosilicates, borosilicates, metal oxides such as alumina and nickel oxide, various clays, nitrocellulose, or nylon. In all embodiments a glass substrate is preferred. According to the invention, probes are selected from the group of nucleic acids including, but not limited to: DNA, genomic DNA, cDNA, and oligonucleotides; and may be natural or synthetic. Oligonucleotide probes preferably are 20 to 25-mer oligonucleotides and DNA/cDNA probes preferably are 500 to 5000 bases in length, although other lengths may be used. Appropriate probe length may be determined by one of ordinary skill in the art by following art-known procedures. In one embodiment, preferred nucleic acid probes are sets of two or more of the OY-TES-1 nucleic acid molecules set forth herein. Preferred protein microarray probes include OY-TES-1 protein (SEQ ID NO:24) and fragments thereof. Probes may be purified to remove contaminants using standard methods known to those of ordinary skill in the art such as gel filtration or precipitation.

In one embodiment, the microarray substrate may be coated with a compound to enhance synthesis of the probe on the substrate. Such compounds include, but are not limited to, oligoethylene glycols. In another embodiment, coupling agents or groups on the substrate can be used to covalently link the first nucleotide or oligonucleotide to the substrate. These agents or groups may include, for example, amino, hydroxy, bromo, and carboxy groups. These reactive groups are preferably attached to the substrate through a hydrocarbyl radical such as an alkylene or phenylene divalent radical, one valence position occupied by the chain bonding and the remaining attached to the reactive groups. These hydrocarbyl groups may contain up to about ten carbon atoms, preferably up to about six carbon atoms. Alkylene radicals are usually preferred containing two to four carbon atoms in the principal chain. These and additional details of the process are disclosed, for example, in U.S. Pat. No. 4,458,066, which is incorporated by reference in its entirety.

In one embodiment, probes are synthesized directly on the substrate in a predetermined grid pattern using methods such as light-directed chemical synthesis, photochemical deprotection, or delivery of nucleotide precursors to the substrate and subsequent probe production.

In another embodiment, the substrate may be coated with a compound to enhance binding of the probe to the substrate. Such compounds include, but are not limited to: polylysine, amino silanes, amino-reactive silanes (Chipping Forecast, 1999) or chromium. In this embodiment, presynthesized probes are applied to the substrate in a precise, predetermined volume and grid pattern, utilizing a computer-controlled robot to apply probe to the substrate in a contact-printing manner or in a non-contact manner such as ink jet or piezo-electric delivery. Probes may be covalently linked to the substrate with methods that include, but are not limited to, UV-irradiation. In another embodiment probes are linked to the substrate with heat.

Targets are nucleic acids, including but not limited to: DNA, genomic DNA, cDNA, RNA, mRNA and may be natural or synthetic. In all embodiments, nucleic acid target molecules from human tissue are preferred. The tissue may be obtained from a subject or may be grown in culture (e.g. from a cancer cell line).

In embodiments of the invention one or more control polypeptide or nucleic acid molecules are attached to the substrate. Preferably, control polypeptide or nucleic acid molecules allow determination of factors such as polypeptide or nucleic acid quality and binding characteristics, reagent quality and effectiveness, binding success, and analysis thresholds and success. Control polypeptide or nucleic acids may include but are not limited to expression products of genes such as housekeeping genes or fragments thereof.

The invention also permits the construction of cancer associated antigen gene “knock-outs” and transgenics in cells and in animals, providing materials for studying certain aspects of cancer and immune system responses to cancer.

The invention also provides isolated polypeptides (including whole proteins and partial proteins) encoded by the foregoing cancer associated antigen nucleic acids. Such polypeptides are useful, for example, alone or as fusion proteins to generate antibodies, as components of an immunoassay or diagnostic assay or as therapeutics. Cancer associated antigen polypeptides can be isolated from biological samples including tissue or cell homogenates, and can also be expressed recombinantly in a variety of prokaryotic and eukaryotic expression systems by constructing an expression vector appropriate to the expression system, introducing the expression vector into the expression system, and isolating the recombinantly expressed protein. Short polypeptides, including protein fragments and antigenic peptides (such as are presented by MHC molecules on the surface of a cell for immune recognition) also can be synthesized chemically using well-established methods of peptide synthesis.

A unique fragment of a cancer associated antigen polypeptide, in general, has the features and characteristics of unique fragments as discussed above in connection with nucleic acids. As will be recognized by those skilled in the art, the size of the unique fragment will depend upon factors such as whether the fragment constitutes a portion of a conserved protein domain. Thus, some regions of cancer associated antigens will require longer segments to be unique while others will require only short segments, typically between 5 and 12 amino acids (e.g. 5, 6, 7, 8, 9, 10, 11 or 12 or more amino acids including each integer up to the full length).

Human homologs of cancer associated antigen polypeptides are related in sequence to the cancer associated antigens described herein, and preferably are also related in function. Preferably the homologs are 90% or more identical to one or more portions of the amino acid sequence of the cancer associated antigens, more preferably are 95% or more identical, and still more preferably are 99% or more identical. Most preferably, the homologs contains at least one fragment of 10 or more amino acids that are identical to the corresponding amino acids of the cancer associated antigens. In some embodiments a human homolog has the same or similar activity or function as a cancer associated antigen. Activities and functions include, but are not limited to, enzymatic activity, recognition by antibodies, DNA binding activity, transcriptional activity, binding to MHC molecules, and the like.

Unique fragments and human homologs of a polypeptide preferably are those fragments and homologs which retain a distinct functional capability of the polypeptide. Functional capabilities which can be retained in a fragment, preferably an unique fragment, of a polypeptide include interaction with antibodies, interaction with other polypeptides or fragments thereof, selective binding of nucleic acids or proteins, and enzymatic activity. One important activity is the ability to act as a signature for identifying the polypeptide. Another is the ability to complex with MHC and to provoke in a mammal, preferably a human, an immune response. Those skilled in the art are well versed in methods for selecting unique amino acid sequences, typically on the basis of the ability of the unique fragment to selectively distinguish the sequence of interest from non-family members. A comparison of the sequence of the fragment to those on known databases typically is all that is necessary.

The invention embraces variants of the cancer associated antigen polypeptides described above. As used herein, a “variant” of a cancer associated antigen polypeptide is a polypeptide which contains one or more modifications to the primary amino acid sequence of a cancer associated antigen polypeptide. Modifications which create a cancer associated antigen variant can be made to a cancer associated antigen polypeptide 1) to reduce or eliminate an activity of a cancer associated antigen polypeptide; 2) to enhance a property of a cancer associated antigen polypeptide, such as protein stability in an expression system or the stability of protein-protein binding; 3) to provide a novel activity or property to a cancer associated antigen polypeptide, such as addition of an antigenic epitope or addition of a detectable moiety; or 4) to provide equivalent or better binding to a MHC molecule. Modifications to a cancer associated antigen polypeptide are typically made to the nucleic acid which encodes the cancer associated antigen polypeptide, and can include deletions, point mutations, truncations, amino acid substitutions and additions of amino acids or non-amino acid moieties. Alternatively, modifications can be made directly to the polypeptide, such as by cleavage, addition of a linker molecule, addition of a detectable moiety, such as biotin, addition of a fatty acid, and the like. Modifications also embrace fusion proteins comprising all or part of the cancer associated antigen amino acid sequence. One of skill in the art will be familiar with methods for predicting the effect on protein conformation of a change in protein sequence, and can thus “design” a variant cancer associated antigen polypeptide according to known methods. One example of such a method is described by Dahiyat and Mayo in Science 278:82-87, 1997, whereby proteins can be designed de novo. The method can be applied to a known protein to vary a only a portion of the polypeptide sequence. By applying the computational methods of Dahiyat and Mayo, specific variants of a cancer associated antigen polypeptide can be proposed and tested to determine whether the variant retains a desired conformation.

In general, variants include cancer associated antigen polypeptides which are modified specifically to alter a feature of the polypeptide unrelated to its desired physiological activity. For example, cysteine residues can be substituted or deleted to prevent unwanted disulfide linkages. Similarly, certain amino acids can be changed to enhance expression of a cancer associated antigen polypeptide by eliminating proteolysis by proteases in an expression system (e.g., dibasic amino acid residues in yeast expression systems in which KEX2 protease activity is present).

Mutations of a nucleic acid which encode a cancer associated antigen polypeptide preferably preserve the amino acid reading frame of the coding sequence, and preferably do not create regions in the nucleic acid which are likely to hybridize to form secondary structures, such a hairpins or loops, which can be deleterious to expression of the variant polypeptide.

Mutations can be made by selecting an amino acid substitution, or by random mutagenesis of a selected site in a nucleic acid which encodes the polypeptide. Variant polypeptides are then expressed and tested for one or more activities to determine which mutation provides a variant polypeptide with the desired properties. Further mutations can be made to variants (or to non-variant cancer associated antigen polypeptides) which are silent as to the amino acid sequence of the polypeptide, but which provide preferred codons for translation in a particular host. The preferred codons for translation of a nucleic acid in, e.g., E. coli, are well known to those of ordinary skill in the art. Still other mutations can be made to the noncoding sequences of a cancer associated antigen gene or cDNA clone to enhance expression of the polypeptide. The activity of variants of cancer associated antigen polypeptides can be tested by cloning the gene encoding the variant cancer associated antigen polypeptide into a bacterial or mammalian expression vector, introducing the vector into an appropriate host cell, expressing the variant cancer associated antigen polypeptide, and testing for a functional capability of the cancer associated antigen polypeptides as disclosed herein. For example, the variant cancer associated antigen polypeptide can be tested for reaction with autologous or allogeneic sera as described in the Examples. Preparation of other variant polypeptides may favor testing of other activities, as will be known to one of ordinary skill in the art.

The skilled artisan will also realize that conservative amino acid substitutions may be made in cancer associated antigen polypeptides to provide functionally equivalent variants of the foregoing polypeptides, i.e, the variants retain the functional capabilities of the cancer associated antigen polypeptides. As used herein, a “conservative amino acid substitution” refers to an amino acid substitution which does not alter the relative charge or size characteristics of the protein in which the amino acid substitution is made. Variants can be prepared according to methods for altering polypeptide sequence known to one of ordinary skill in the art such as are found in references which compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Exemplary functionally equivalent variants of the cancer associated antigen polypeptides include conservative amino acid substitutions of in the amino acid sequences of proteins disclosed herein. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

For example, upon determining that a peptide derived from a cancer associated antigen polypeptide is presented by an MHC molecule and recognized by antibodies or CTLs, one can make conservative amino acid substitutions to the amino acid sequence of the peptide, particularly at residues which are thought not to be direct contact points with the MHC molecule. For example, methods for identifying functional variants of HLA class II binding peptides are provided in a published PCT application of Strominger and Wucherpfennig (PCT/US96/03182). Peptides bearing one or more amino acid substitutions also can be tested for concordance with known HLA/MHC motifs prior to synthesis using, e.g. the computer program described by D'Amaro and Drijfhout (D'Amaro et al., Human Immunol. 43:13-18, 1995; Drijfhout et al., Human Immunol. 43:1-12, 1995). The substituted peptides can then be tested for binding to the MHC molecule and recognition by antibodies or CTLs when bound to MHC. These variants can be tested for improved stability and are useful, inter alia, in vaccine compositions.

Conservative amino-acid substitutions in the amino acid sequence of cancer associated antigen polypeptides to produce functionally equivalent variants of cancer associated antigen polypeptides typically are made by alteration of a nucleic acid encoding a cancer associated antigen polypeptide. Such substitutions can be made by a variety of methods known to one of ordinary skill in the art. For example, amino acid substitutions may be made by PCR-directed mutation, site-directed mutagenesis according to the method of Kunkel (Kunkel, Proc. Nat. Acad. Sci. U.S.A. 82: 488-492, 1985), or by chemical synthesis of a gene encoding a cancer associated antigen polypeptide. Where amino acid substitutions are made to a small unique fragment of a cancer associated antigen polypeptide, such as an antigenic epitope recognized by autologous or allogeneic sera or cytolytic T lymphocytes, the substitutions can be made by directly synthesizing the peptide. The activity of functionally equivalent fragments of cancer associated antigen polypeptides can be tested by cloning the gene encoding the altered cancer associated antigen polypeptide into a bacterial or mammalian expression vector, introducing the vector into an appropriate host cell, expressing the altered cancer associated antigen polypeptide, and testing for a functional capability of the cancer associated antigen polypeptides as disclosed herein. Peptides which are chemically synthesized can be tested directly for function, e.g., for binding to antisera recognizing associated antigens.

The invention as described herein has a number of uses, some of which are described elsewhere herein. First, the invention permits isolation of the cancer associated antigen protein molecules. A variety of methodologies well-known to the skilled practitioner can be utilized to obtain isolated cancer associated antigen molecules. The polypeptide may be purified from cells which naturally produce the polypeptide by chromatographic means or immunological recognition. Alternatively, an expression vector may be introduced into cells to cause production of the polypeptide. In another method, mRNA transcripts may be microinjected or otherwise introduced into cells to cause production of the encoded polypeptide. Translation of mRNA in cell-free extracts such as the reticulocyte lysate system also may be used to produce polypeptide. Those skilled in the art also can readily follow known methods for isolating cancer associated antigen polypeptides. These include, but are not limited to, immunochromatography, HPLC, size-exclusion chromatography, ion-exchange chromatography and immune-affinity chromatography.

Identification of the cancer associated antigens also permits the identification and isolation of related human sequences (e.g. human homologs such as SEQ ID NOs:23 and 24), including immunogenic fragments of polypeptides and nucleic acids which encode such fragments. The isolation of homologs of different species is described elsewhere herein, and exemplified in Example 8.

The isolation and identification of cancer associated antigen genes also makes it possible for the artisan to diagnose a disorder characterized by expression of cancer associated antigens. These methods involve determining expression of one or more cancer associated antigen nucleic acids, and/or encoded cancer associated antigen polypeptides and/or peptides derived therefrom. In the former situation, such determinations can be carried out via any standard nucleic acid determination assay, including the polymerase chain reaction, or assaying with labeled hybridization probes. In the latter situation, such determinations can be carried out by screening patient antisera for recognition of the polypeptide.

The invention also makes it possible isolate proteins which bind to cancer associated antigens as disclosed herein, including antibodies and cellular binding partners of the cancer associated antigens. Additional uses are described further herein.

Expression of cancer associated nucleic acid molecules can also be determined using protein measurement methods to determine expression of SEQ ID NOs: 1, 3, 5, 7, 9, 11, 13, 15, 17, 19 and 23, e.g., by determining the expression of polypeptides encoded by these nucleic acid molecules (SEQ ID NOs: 2, 4, 6, 8, 10, 12, 14, 16, 18, 20, 22 and 24). Preferred methods of specifically and quantitatively measuring proteins include, but are not limited to: protein microarrays, mass spectroscopy-based methods such as surface enhanced laser desorption ionization (SELDI; e.g., Ciphergen ProteinChip System), non-mass spectroscopy-based methods, and immunohistochemistry-based methods such as 2-dimensional gel electrophoresis.

Protein microarrays are similar in concept to the nucleic acid microarrays described above. Proteins are spotter or printed on a substrate and exposed to a sample to determine interactions of molecules with the proteins fixed on the substrate. For example, MacBeath and Schrieiber described a protein microarray system and several of its uses. See, Science 289:1760-1763, 2000. In one embodiment of the invention, cancer-associated proteins are spotted on a substrate in a microarray and the microarray is used to determine the presence of proteins in a biological sample that bind to the cancer associated proteins. This type of assay permits rapid determination of the amount and kind of binding proteins expressed, such as antibodies present in a cancer patient's blood.

SELDI methodology may, through procedures known to those of ordinary skill in the art, be used to vaporize microscopic amounts of tumor tissue and to create a “fingerprint” of individual proteins, thereby allowing simultaneous measurement of the abundance of many proteins in a single sample. Preferably SELDI-based assays may be utilized to identify and/or classify tumors. Such assays preferably include, but are not limited to the following examples. Gene products that are encoded by nucleic acids analyzed by nucleic acid microarrays may be selectively measured by specific (antibody mediated) capture to the SELDI protein disc (e.g., selective SELDI). Gene products discovered by protein screening (e.g., with 2-D gels), may be resolved by “total protein SELDI” optimized to visualize the expression of those genes of interest from among the nucleic acid molecules described herein.

SELDI measurement of multiple markers from among the genes disclosed herein may be utilized for tumor diagnosis and/or classification. SELDI also can be used diagnostically or prognostically for analysis of precancerous tissues to determine the risk of cancer based on SELDI results. The protein analysis methods also can be used as a prognostic method for selecting treatment strategies for cancer patients, based on the particular pattern and amounts of gene expression in a patient's tumor.

The invention also provides, in certain embodiments, “dominant negative” polypeptides derived from cancer associated antigen polypeptides. A dominant negative polypeptide is an inactive variant of a protein, which, by interacting with the cellular machinery, displaces an active protein from its interaction with the cellular machinery or competes with the active protein, thereby reducing the effect of the active protein. For example, a dominant negative receptor which binds a ligand but does not transmit a signal in response to binding of the ligand can reduce the biological effect of expression of the ligand. Likewise, a dominant negative catalytically-inactive kinase which interacts normally with target proteins but does not phosphorylate the target proteins can reduce phosphorylation of the target proteins in response to a cellular signal. Similarly, a dominant negative transcription factor which binds to a promoter site in the control region of a gene but does not increase gene transcription can reduce the effect of a normal transcription factor by occupying promoter binding sites without increasing transcription.

The end result of the expression of a dominant negative polypeptide in a cell is a reduction in function of active proteins. One of ordinary skill in the art can assess the potential for a dominant negative variant of a protein, and using standard mutagenesis techniques to create one or more dominant negative variant polypeptides. For example, given the teachings contained herein of cancer associated antigens, especially those which are similar to known proteins which have known activities (e.g., OY-MC-4 (pem)), one of ordinary skill in the art can modify the sequence of the cancer associated antigens by site-specific mutagenesis, scanning mutagenesis, partial gene deletion or truncation, and the like. See, e.g., U.S. Pat. No. 5,580,723 and Sambrook et al., Molecular Cloning: A Laboratory Manual, Second Edition, Cold Spring Harbor Laboratory Press, 1989. The skilled artisan then can test the population of mutagenized polypeptides for diminution in a selected and/or for retention of such an activity. Other similar methods for creating and testing dominant negative variants of a protein will be apparent to one of ordinary skill in the art.

The invention also involves agents such as polypeptides which bind to cancer associated antigen polypeptides. Such binding agents can be used, for example, in screening assays to detect the presence or absence of cancer associated antigen polypeptides and complexes of cancer associated antigen polypeptides and their binding partners and in purification protocols to isolated cancer associated antigen polypeptides and complexes of cancer associated antigen polypeptides and their binding partners. Such agents also can be used to inhibit the native activity of the cancer associated antigen polypeptides, for example, by binding to such polypeptides.

The invention, therefore, embraces peptide binding agents which, for example, can be antibodies or fragments of antibodies having the ability to selectively bind to cancer associated antigen polypeptides. Antibodies include polyclonal and monoclonal antibodies, prepared according to conventional methodology.

Significantly, as is well-known in the art, only a small portion of an antibody molecule, the paratope, is involved in the binding of the antibody to its epitope (see, in general, Clark, W. R. (1986) The Experimental Foundations of Modem Immunology Wiley & Sons, Inc., New York; Roitt, I. (1991) Essential Immunology, 7th Ed., Blackwell Scientific Publications, Oxford). The pFc′ and Fc regions, for example, are effectors of the complement cascade but are not involved in antigen binding. An antibody from which the pFc′ region has been enzymatically cleaved, or which has been produced without the pFc′ region, designated an F(ab′)₂ fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDR3). The CDRs, and in particular the CDR3 regions, and more particularly the heavy chain CDR3, are largely responsible for antibody specificity.

It is now well-established in the art that the non-CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody. See, e.g., U.S. Pat. Nos. 4,816,567, 5,225,539, 5,585,089, 5,693,762 and 5,859,205.

Thus, for example, PCT International Publication Number WO 92/04381 teaches the production and use of humanized murine RSV antibodies in which at least a portion of the murine FR regions have been replaced by FR regions of human origin. Such antibodies, including fragments of intact antibodies with antigen-binding ability, are often referred to as “chimeric” antibodies.

Thus, as will be apparent to one of ordinary skill in the art, the present invention also provides for F(ab′)₂, Fab, Fv and Fd fragments; chimeric antibodies in which the Fc and/or FR and/or CDR1 and/or CDR2 and/or light chain CDR3 regions have been replaced by homologous human or non-human sequences; chimeric F(ab′)₂ fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non-human sequences. The present invention also includes so-called single chain antibodies.

Thus, the invention involves polypeptides of numerous size and type that bind specifically to cancer associated antigen polypeptides, and complexes of both cancer associated-antigen polypeptides and their binding partners. These polypeptides may be derived also from sources other than antibody technology. For example, such polypeptide binding agents can be provided by degenerate peptide libraries which can be readily prepared in solution, in immobilized form or as phage display libraries. Combinatorial libraries also can be synthesized of peptides containing one or more amino acids. Libraries further can be synthesized of peptoids and non-peptide synthetic moieties.

Phage display can be particularly effective in identifying binding peptides useful according to the invention. Briefly, one prepares a phage library (using e.g. m13, fd, or lambda phage), displaying inserts from 4 to about 80 amino acid residues using conventional procedures. The inserts may represent, for example, a completely degenerate or biased array. One then can select phage-bearing inserts which bind to the cancer associated antigen polypeptide. This process can be repeated through several cycles of reselection of phage that bind to the cancer associated antigen polypeptide. Repeated rounds lead to enrichment of phage bearing particular sequences. DNA sequence analysis can be conducted to identify the sequences of the expressed polypeptides. The minimal linear portion of the sequence that binds to the cancer associated antigen polypeptide can be determined. One can repeat the procedure using a biased library containing inserts containing part or all of the minimal linear portion plus one or more additional degenerate residues upstream or downstream thereof. Yeast two-hybrid screening methods also may be used to identify polypeptides that bind to the cancer associated antigen polypeptides. Thus, the cancer associated antigen polypeptides of the invention, or a fragment thereof, can be used to screen peptide libraries, including phage display libraries, to identify and select peptide binding partners of the cancer associated antigen polypeptides of the invention. Such molecules can be used, as described, for screening assays, for purification protocols, for interfering directly with the functioning of cancer associated antigen and for other purposes that will be apparent to those of ordinary skill in the art.

As detailed herein, the foregoing antibodies and other binding molecules may be used for example to identify tissues expressing protein or to purify protein. Antibodies also may be coupled to specific diagnostic labeling agents for imaging of cells and tissues that express cancer associated antigens or to therapeutically useful agents according to standard coupling procedures. Diagnostic agents include, but are not limited to, barium sulfate, iocetamic acid, iopanoic acid, ipodate calcium, diatrizoate sodium, diatrizoate meglumine, metrizamide, tyropanoate-sodium and radiodiagnostics including positron emitters such as fluorine-18 and carbon-11, gamma emitters such as iodine-123, technitium-99m, iodine-131 and indium-111, nuclides for nuclear-magnetic resonance such as fluorine and gadolinium. Other diagnostic agents useful in the invention will be apparent to one of ordinary skill in the art. As used herein, “therapeutically useful agents” include any therapeutic molecule which desirably is targeted selectively to a cell expressing one of the cancer antigens disclosed herein, including antineoplastic agents, radioiodinated compounds, toxins, other cytostatic or cytolytic drugs, and so forth. Antineoplastic therapeutics are well known and include: aminoglutethimide, azathioprine, bleomycin sulfate, busulfan, carmustine, chlorambucil, cisplatin, cyclophosphamide, cyclosporine, cytarabidine, dacarbazine, dactinomycin, daunorubicin, doxorubicin, taxol, etoposide, fluorouracil, interferon-α, lomustine, mercaptopurine, methotrexate, mitotane, procarbazine HCl, thioguanine, vinblastine sulfate and vincristine sulfate. Additional antineoplastic agents include those disclosed in Chapter 52, Antineoplastic Agents (Paul Calabresi and Bruce A. Chabner), and the introduction thereto, 1202-1263, of Goodman and Gilman's “The Pharmacological Basis of Therapeutics”, Eighth Edition, 1990, McGraw-Hill, Inc. (Health Professions Division). Toxins can be proteins such as, for example, pokeweed anti-viral protein, cholera toxin, pertussis toxin, ricin, gelonin, abrin, diphtheria exotoxin, or Pseudomonas exotoxin. Toxin moieties can also be high energy-emitting radionuclides such as cobalt-60.

In the foregoing methods, antibodies prepared according to the invention also preferably are specific for the cancer associated antigen/MHC complexes described herein.

When “disorder” is used herein, it refers to any pathological condition where the cancer associated antigens are expressed. An example of such a disorder is cancer, with fibrosarcoma as a particular example. For human cancers, additional particular examples include bladder cancer, breast cancer, lung cancer, colon cancer, and hepatoma.

Samples of tissue and/or cells for use in the various methods described herein can be obtained through standard methods such as tissue biopsy, including punch biopsy and cell scraping, and collection of blood or other bodily fluids by aspiration or other methods.

In certain embodiments of the invention, an immunoreactive cell sample is removed from a subject. By “immunoreactive cell” is meant a cell which can mature into an immune cell (such as a B cell, a helper T cell, or a cytolytic T cell) upon appropriate stimulation. Thus immunoreactive cells include CD34⁺ hematopoietic stem cells, immature T cells and immature B cells. When it is desired to produce cytolytic T cells which recognize a cancer associated antigen, the immunoreactive cell is contacted with a cell which expresses a cancer associated antigen under conditions favoring production, differentiation and/or selection of cytolytic T cells; the differentiation of the T cell precursor into a cytolytic T cell upon exposure to antigen is similar to clonal selection of the immune system.

Some therapeutic approaches based upon the disclosure are premised on a response by a subject's immune system, leading to lysis of antigen presenting cells, such as cancer cells which present one or more cancer associated antigens. One such-approach is the administration of autologous CTLs specific to a cancer associated antigen/MHC complex to a subject with abnormal cells of the phenotype at issue. It is within the ability of one of ordinary skill in the art to develop such CTLs in vitro. An example of a method for T cell differentiation is presented in International Application number PCT/US96/05607. Generally, a sample of cells taken from a subject, such as blood cells, are contacted with a cell presenting the complex and capable of provoking CTLs to proliferate. The target cell can be a transfectant, such as a COS cell. These transfectants present the desired complex of their surface and, when combined with a CTL of interest, stimulate its proliferation. COS cells are widely available, as are other suitable host cells. Specific production of CTL clones is well known in the art. The clonally expanded autologous CTLs then are administered to the subject.

Another method for selecting antigen-specific CTL clones has recently been described (Altman et al., Science 274:94-96, 1996; Dunbar et al., Curr. Biol. 8:413-416, 1998), in which fluorogenic tetramers of MHC class I molecule/peptide complexes are used to detect specific CTL clones. Briefly, soluble MHC class I molecules are folded in vitro in the presence of β₂-microglobulin and a peptide antigen which binds the class I molecule. After purification, the MHC/peptide complex is purified and labeled with biotin. Tetramers are formed by mixing the biotinylated peptide-MHC complex with labeled avidin (e.g. phycoerythrin) at a molar ratio or 4:1. Tetramers are then contacted with a source of CTLs such as peripheral blood or lymph node. The tetramers bind CTLs which recognize the peptide antigen/MHC class I complex. Cells bound by the tetramers can be sorted by fluorescence activated cell sorting to isolate the reactive CTLs. The isolated CTLs then can be expanded in vitro for use as described herein.

To detail a therapeutic methodology, referred to as adoptive transfer (Greenberg, J. Immunol. 136(5): 1917, 1986; Riddel et al., Science 257: 238, 1992; Lynch et al, Eur. J. Immunol. 21:1403-1410, 1991; Kast et al., Cell 59: 603-614, 1989), cells presenting the desired complex (e.g., dendritic cells) are combined with CTLs leading to proliferation of the CTLs specific thereto. The proliferated CTLs are then administered to a subject with a cellular abnormality which is characterized by certain of the abnormal cells presenting the particular complex. The CTLs then lyse the abnormal cells, thereby achieving the desired therapeutic goal.

The foregoing therapy assumes that at least some of the subject's abnormal cells present the relevant HLA/cancer associated antigen complex. This can be determined very easily, as the art is very familiar with methods for identifying cells which present a particular HLA molecule, as well as how to identify cells expressing DNA of the pertinent sequences, in this case a cancer associated antigen sequence. Once cells presenting the relevant complex are identified via the foregoing screening methodology, they can be combined with a sample from a patient, where the sample contains CTLs. If the complex presenting cells are lysed by the mixed CTL sample, then it can be assumed that a cancer associated antigen is being presented, and the subject is an appropriate candidate for the therapeutic approaches set forth herein.

Adoptive transfer is not the only form of therapy that is available in accordance with the invention. CTLs can also be provoked in vivo, using a number of approaches. One approach is the use of non-proliferative cells expressing the complex. The cells used in this approach may be those that normally express the complex, such as irradiated tumor cells or cells transfected with one or both of the genes necessary for presentation of the complex (i.e. the antigenic peptide and the presenting MHC molecule). Chen et al. (Proc. Natl. Acad. Sci. USA 88:110-114, 1991) exemplifies this approach, showing the use of transfected cells expressing HPV E7 peptides in a therapeutic regime. Various cell types may be used. Similarly, vectors carrying one or both of the genes of interest may be used. Viral or bacterial vectors are especially preferred. For example, nucleic acids which encode a cancer associated antigen polypeptide or peptide may be operably linked to promoter and enhancer sequences which direct expression of the cancer associated antigen polypeptide or peptide in certain tissues or cell types. The nucleic acid may be incorporated into an expression vector. Expression vectors may be unmodified extrachromosomal nucleic acids, plasmids or viral genomes constructed or modified to enable insertion of exogenous nucleic acids, such as those encoding cancer associated antigen, as described elsewhere herein. Nucleic acids encoding a cancer associated antigen also may be inserted into a retroviral genome, thereby facilitating integration of the nucleic acid into the genome of the target tissue or cell type. In these systems, the gene of interest is carried by a microorganism, e.g., a vaccinia virus, pox virus, herpes simplex virus, retrovirus or adenovirus, and the materials de facto “infect” host cells. The cells which result present the complex of interest, and are recognized by autologous CTLs, which then proliferate.

A similar effect can be achieved by combining the cancer associated antigen or a stimulatory fragment thereof with an adjuvant to facilitate incorporation into antigen presenting cells in vivo. The cancer associated antigen polypeptide is processed to yield the peptide partner of the MHC molecule while a cancer associated antigen peptide may be presented without the need for further processing. Generally, subjects can receive an intradermal injection of an effective amount of the cancer associated antigen. Initial doses can be followed by booster doses, following immunization protocols standard in the art. Preferred cancer associated antigens include those found to react with allogeneic cancer antisera, shown in the examples below.

The invention involves the use of various materials disclosed herein to “immunize” subjects or as “vaccines”. As used herein, “immunization” or “vaccination” means increasing or activating an immune response against an antigen. It does not require elimination or eradication of a condition but rather contemplates the clinically favorable enhancement of an immune response toward an antigen. Generally accepted animal models, including the MethA fibroasrcoma model used herein, can be used for testing of immunization against cancer using a cancer associated antigen nucleic acid. For example, human cancer cells can be introduced into a mouse to create a tumor, and one or more cancer associated antigen nucleic acids can be delivered by the methods described herein. The effect on the cancer cells (e.g., reduction of tumor size) can be assessed as a measure of the effectiveness of the cancer associated antigen nucleic acid immunization. Of course, testing of the foregoing animal model using more conventional methods for immunization include the administration of one or more cancer associated antigen polypeptides or peptides derived therefrom, optionally combined with one or more adjuvants and/or cytokines to boost the immune response. Methods for immunization, including formulation of a vaccine composition and selection of doses, route of administration and the schedule of administration (e.g. primary and one or more booster doses), are well known in the art. The tests also can be performed in humans, where the end point is to test for the presence of enhanced levels of circulating CTLs against cells bearing the antigen, to test for levels of circulating antibodies against the antigen, to test for the presence of cells expressing the antigen and so forth.

As part of the immunization compositions, one or more cancer associated antigens or stimulatory fragments thereof are administered with one or more adjuvants to induce an immune response or to increase an immune response. An adjuvant is a substance incorporated into or administered with antigen which potentiates the immune response. Adjuvants may enhance the immunological response by providing a reservoir of antigen (extracellularly or within macrophages), activating macrophages and stimulating specific sets of lymphocytes. Adjuvants of many kinds are well known in the art. Specific examples of adjuvants include monophosphoryl lipid A (MPL, SmithKline Beecham), a congener obtained after purification and acid hydrolysis of Salmonella minnesota Re 595 lipopolysaccharide; saponins including QS21 (SmithKline Beecham), a pure QA-21 saponin purified from Quillja saponaria extract; DQS21, described in PCT application WO96/33739 (SmithKline Beecham); QS-7, QS-17, QS-18, and QS-LI (So et al., Mol. Cells 7:178-186, 1997); incomplete Freund's adjuvant; complete Freund's adjuvant; montanide; alum; CpG oligonucleotides (see e.g. Kreig et al., Nature 374:546-9, 1995); and various water-in-oil emulsions prepared from biodegradable oils such as squalene and/or tocopherol. Preferably, the peptides are administered mixed with a combination of DQS21/MPL. The ratio of DQS21 to MPL typically will be about 1:10 to 10:1, preferably about 1:5 to 5:1 and more preferably about 1:1. Typically for human administration, DQS21 and MPL will be present in a vaccine formulation in the range of about 1 μg to about 100 μg. Other adjuvants are known in the art and can be used in the invention (see, e.g. Goding, Monoclonal Antibodies: Principles and Practice, 2nd Ed., 1986). Methods for the preparation of mixtures or emulsions of peptide and adjuvant are well known to those of skill in the art of vaccination.

Other agents which stimulate the immune response of the subject can also be administered to the subject. For example, other cytokines are also useful in vaccination protocols as a result of their lymphocyte regulatory properties. Many other cytokines useful for such purposes will be known to one of ordinary skill in the art, including interleukin-12 (IL-12) which has been shown to enhance the protective effects of vaccines (see, e.g. Science 268: 1432-1434, 1995), GM-CSF and IL-18. Thus cytokines can be administered in conjunction with antigens and adjuvants to increase the immune response to the antigens.

There are a number of immune response potentiating compounds that can be used in vaccination protocols. These include costimulatory molecules provided in either protein or nucleic acid form. Such costimulatory molecules include the B7-1 and B7-2 (CD80 and CD86 respectively) molecules which are expressed on dendritic cells (DC) and interact with the CD28 molecule expressed on the T cell. This interaction provides costimulation (signal 2) to an antigen/MHC/TCR stimulated (signal 1) T cell, increasing T cell proliferation and effector function. B7 also interacts with CTLA4 (CD152) on T cells and studies involving CTLA4 and B7 ligands indicate that the B7-CTLA4 interaction can enhance antitumor immunity and CTL proliferation (Zheng P., et al. Proc. Natl. Acad. Sci. USA 95 (11):6284-6289 (1998)).

B7 typically is not expressed on tumor cells so they are not efficient antigen presenting cells (APCs) for T cells. Induction of B7 expression would enable the tumor cells to stimulate more efficiently CTL proliferation and effector function. A combination of B7/IL-6 μL-12 costimulation has been shown to induce IFN-gamma and a Th1 cytokine profile in the T cell population leading to further enhanced T cell activity (Gajewski et al., J Immunol, 154:5637-5648 (1995)). Tumor cell transfection with B7 has ben discussed in relation to in vitro CTL expansion for adoptive transfer immunotherapy by Wang et al., (J. Immunol., 19:1-8 (1986)). Other delivery mechanisms for the B7 molecule would include nucleic acid (naked DNA) immunization (Kim J., et al. Nat. Biotechnol., 15:7:641-646 (1997)) and recombinant viruses such as adeno and pox (Wendtner et al., Gene Ther., 4:7:726-735 (1997)). These systems are all amenable to the construction and use of expression cassettes for the coexpression of B7 with other molecules of choice such as the antigens or fragment(s) of antigens discussed herein (including polytopes) or cytokines. These delivery systems can be used for induction of the appropriate molecules in vitro and for in vivo vaccination situations. The use of anti-CD28 antibodies to directly stimulate T cells in vitro and in vivo could also be considered. Similarly, the inducible co-stimulatory molecule ICOS which induces T cell responses to foreign antigen could be modulated, for example, by use of anti-ICOS antibodies (Hutloff et al., Nature 397:263-266, 1999).

Lymphocyte function associated antigen-3 (LFA-3) is expressed on APCs and some tumor cells and interacts with CD2 expressed on T cells. This interaction induces T cell IL-2 and IFN-gamma production and can thus complement but not substitute, the B7/CD28 costimulatory interaction (Parra et al., J. Immunol., 158:637-642 (1997), Fenton et al., J Immunother., 21:2:95-108 (1998)).

Lymphocyte function associated antigen-1 (LFA-1) is expressed on leukocytes and interacts with ICAM-1 expressed on APCs and some tumor cells. This interaction induces T cell IL-2 and IFN-gamma production and can thus complement but not substitute, the B7/CD28 costimulatory interaction (Fenton et al., J Immunother., 21:2:95-108 (1998)). LFA-1 is thus a further example of a costimulatory molecule that could be provided in a vaccination protocol in the various ways discussed above for B7.

Complete CTL activation and effector function requires Th cell help through the interaction between the Th cell CD40L (CD40 ligand) molecule and the CD40 molecule expressed by DCs (Ridge et al., Nature, 393:474 (1998), Bennett et al., Nature, 393:478 (1998), Schoenberger et al., Nature, 393:480 (1998)). This mechanism of this costimulatory signal is likely to involve upregulation of B7 and associated IL-6/IL-12 production by the DC (APC). The CD40-CD40L interaction thus complements the signal 1 (antigen/MHC-TCR) and signal 2 (B7-CD28) interactions. The use of anti-CD40 antibodies to stimulate DC cells directly, would be expected to enhance a response to tumor antigens which are normally encountered outside of a inflammatory context or are presented by non-professional APCs (tumor cells). In these situations Th help and B7 costimulation signals are not provided. This mechanism might be used in the context of antigen pulsed DC based therapies or in situations where Th epitopes have not been defined within known TRA precursors.

A cancer associated antigen polypeptide, or a fragment thereof, also can be used to isolate their native binding partners. Isolation of such binding partners may be performed according to well-known methods. For example, isolated cancer associated antigen polypeptides can be attached to a substrate (e.g., chromatographic media, such as polystyrene beads, or a filter), and then a solution suspected of containing the binding partner may be applied to the substrate. If a binding partner which can interact with cancer associated antigen polypeptides is present in the solution, then it will bind to the substrate-bound cancer associated antigen polypeptide. The binding partner then may be isolated.

It will also be recognized that the invention embraces the use of the cancer associated antigen cDNA sequences in expression vectors, as well as to transfect host cells and cell lines, be these prokaryotic (e.g., E. coli), or eukaryotic (e.g., dendritic cells, B cells, CHO cells, COS cells, yeast expression systems and recombinant baculovirus expression in insect cells). Especially useful are mammalian cells such as human, mouse, hamster, pig, goat, primate, etc. They may be of a wide variety of tissue types, and include primary cells and cell lines. Specific examples include dendritic cells, peripheral blood leukocytes, bone marrow stem cells and embryonic stem cells. The expression vectors require that the pertinent sequence, i.e., those nucleic acids described herein, be operably linked to a promoter.

The invention also contemplates delivery of nucleic acids, polypeptides or peptides for vaccination. Delivery of polypeptides and peptides can be accomplished according to standard vaccination protocols which are well known in the art. In another embodiment, the delivery of nucleic acid is accomplished by ex vivo methods, i.e. by removing a cell from a subject, genetically engineering the cell to include a cancer associated antigen, and reintroducing the engineered cell into the subject. One example of such a procedure is outlined in U.S. Pat. No. 5,399,346 and in exhibits submitted in the file history of that patent, all of which are publicly available documents. In general, it involves introduction in vitro of a functional copy of a gene into a cell(s) of a subject, and returning the genetically engineered cell(s) to the subject. The functional copy of the gene is under operable control of regulatory elements which permit expression of the gene in the genetically engineered cell(s). Numerous transfection and transduction techniques as well as appropriate expression vectors are well known to those of ordinary skill in the art, some of which are described in PCT application WO95/00654. In vivo nucleic acid delivery using vectors such as viruses and targeted liposomes also is contemplated according to the invention.

In preferred embodiments, a virus vector for delivering a nucleic acid encoding a cancer associated antigen is selected from the group consisting of adenoviruses, adeno-associated viruses, poxviruses including vaccinia viruses and attenuated poxviruses, Semliki Forest virus, Venezuelan equine encephalitis virus, retroviruses, Sindbis virus, and Ty virus-like particle. Examples of viruses and virus-like particles which have been used to deliver exogenous nucleic acids include: replication-defective adenoviruses (e.g., Xiang et al., Virology 219:220-227, 1996; Eloit et al., J. Virol. 7:5375-5381, 1997; Chengalvala et al., Vaccine 15:335-339, 1997), a modified retrovirus (Townsend et al., J. Virol. 71:3365-3374, 1997), a nonreplicating retrovirus (Irwin et al., J. Virol. 68:5036-5044, 1994), a replication defective Semliki Forest virus (Zhao et al., Proc. Natl. Acad. Sci. USA 92:3009-3013, 1995), canarypox virus and highly attenuated vaccinia virus derivative (Paoletti, Proc. Natl. Acad. Sci. USA 93:11349-11353, 1996), non-replicative vaccinia virus (Moss, Proc. Natl. Acad. Sci. USA 93:11341-11348, 1996), replicative vaccinia virus (Moss, Dev. Biol. Stand. 82:55-63, 1994), Venzuelan equine encephalitis virus (Davis et al., J. Virol. 70:3781-3787, 1996), Sindbis virus (Pugachev et al., Virology 212:587-594, 1995), and Ty virus-like particle (Allsopp et al., Eur. J Immunol 26:1951-1959, 1996). In preferred embodiments, the virus vector is an adenovirus.

Another preferred virus for certain applications is the adeno-associated virus, a double-stranded DNA virus. The adeno-associated virus is capable of infecting a wide range of cell types and species and can be engineered to be replication-deficient. It further has advantages, such as heat and lipid solvent stability, high transduction frequencies in cells of diverse lineages, including hematopoietic cells, and lack of superinfection inhibition thus allowing multiple series of transductions. The adeno-associated virus can integrate into human cellular DNA in a site-specific manner, thereby minimizing the possibility of insertional mutagenesis and variability of inserted gene expression. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

In general, other preferred viral vectors are based on non-cytopathic eukaryotic viruses in which non-essential genes have been replaced with the gene of interest. Non-cytopathic viruses include retroviruses, the life cycle of which involves reverse transcription of genomic viral RNA into DNA with subsequent proviral integration into host cellular DNA. Adenoviruses and retroviruses have been approved for human gene therapy trials. In general, the retroviruses are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in Kriegler, M., “Gene Transfer and Expression, A Laboratory Manual,” W. H. Freeman Co., New York (1990) and Murry, E. J. Ed. “Methods in Molecular Biology,” vol. 7, Humana Press, Inc., Clifton, N.J. (1991).

Preferably the foregoing nucleic acid delivery vectors: (1) contain exogenous genetic material that can be transcribed and translated in a mammalian cell and that can induce an immune response in a host, and (2) contain on a surface a ligand that selectively binds to a receptor on the surface of a target cell, such as a mammalian cell, and thereby gains entry to the target cell.

Various techniques may be employed for introducing nucleic acids of the invention into cells, depending on whether the nucleic acids are introduced in vitro or in vivo in a host. Such techniques include transfection of nucleic acid-CaPO₄ precipitates, transfection of nucleic acids associated with DEAE, transfection or infection with the foregoing viruses including the nucleic acid of interest, liposome mediated transfection, and the like. For certain uses, it is preferred to target the nucleic acid to particular cells. In such instances, a vehicle used for delivering a nucleic acid of the invention into a cell (e.g., a retrovirus, or other virus; a liposome)-can have a targeting molecule attached thereto. For example, a molecule such as an antibody specific for a surface membrane protein on the target cell or a ligand for a receptor on the target cell can be bound to or incorporated within the nucleic acid delivery vehicle. Preferred antibodies include antibodies which selectively bind a cancer associated antigen, alone or as a complex with a MHC molecule. Especially preferred are monoclonal antibodies. Where liposomes are employed to deliver the nucleic acids of the invention, proteins which bind to a surface membrane protein associated with endocytosis may be incorporated into the liposome formulation for targeting and/or to facilitate uptake. Such proteins include capsid proteins or fragments thereof tropic for a particular cell type, antibodies for proteins which undergo internalization in cycling, proteins that target intracellular localization and enhance intracellular half life, and the like. Polymeric delivery systems also have been used successfully to deliver nucleic acids into cells, as is known by those skilled in the art. Such systems even permit oral delivery of nucleic acids.

When administered, the therapeutic compositions of the present invention can be administered in pharmaceutically acceptable preparations. Such preparations may routinely contain pharmaceutically acceptable concentrations of salt, buffering agents, preservatives, compatible carriers, supplementary immune potentiating agents such as adjuvants and cytokines and optionally other therapeutic agents.

The therapeutics of the invention can be administered by any conventional route, including injection or by gradual infusion over time. The administration may, for example, be oral, intravenous, intraperitoneal, intramuscular, intracavity, subcutaneous, or transdermal. When antibodies are used therapeutically, a preferred route of administration is by pulmonary aerosol. Techniques for preparing aerosol delivery systems containing antibodies are well known to those of skill in the art. Generally, such systems should utilize components which will not significantly impair the biological properties of the antibodies, such as the paratope binding capacity (see, for example, Sciarra and Cutie, “Aerosols,” in Remington's Pharmaceutical Sciences, 18th edition, 1990, pp 1694-1712; incorporated by reference).

Those of skill in the art can readily determine the various parameters and conditions for producing antibody aerosols without resort to undue experimentation. When using antisense preparations of the invention, slow intravenous administration is preferred.

The compositions of the invention are administered in effective amounts. An “effective amount” is that amount of a cancer associated antigen composition that alone, or together with further doses, produces the desired response, e.g. increases an immune response to the cancer associated antigen. In the case of treating a particular disease or condition characterized by expression of one or more cancer associated antigens, such as cancer, the desired response is inhibiting the progression of the disease. This may involve only slowing the progression of the disease temporarily, although more preferably, it involves halting the progression of the disease permanently. This can be monitored by routine methods or can be monitored according to diagnostic methods of the invention discussed herein. The desired response to treatment of the disease or condition also can be delaying the onset or even preventing the onset of the disease or condition.

Monitoring the progression, regression or onset of a condition can include, for example, obtaining samples from a patient or a person suspected of having the condition at sequential times and assaying such samples for the presence and/or amount of protein or nucleic acid markers of the condition. Such markers include the nucleic acids and polypeptides of the invention, antibodies that bind to the polypeptides, nucleic acids that hybridize to the nucleic acids, and other molecules known to be useful in diagnostic applications. Onset of a cancerous condition is indicated by the appearance of marker(s) in a subject's samples where there were no such marker(s) previously. Progression and regression of a cancerous condition are generally indicated by the increase or decrease, respectively, of marker(s) in a subject's samples over time.

Such amounts will depend, of course, on the particular condition being treated, the severity of the condition, the individual patient parameters including age, physical condition, size and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

The pharmaceutical compositions used in the foregoing methods preferably are sterile and contain an effective amount of cancer associated antigen or nucleic acid encoding cancer associated antigen for producing the desired response in a unit of weight or volume suitable for administration to a patient. The response can, for example, be measured by determining the immune response following administration of the cancer associated antigen composition via a reporter system by measuring downstream effects such as gene expression, or by measuring the physiological effects of the cancer associated antigen composition, such as regression of a tumor or decrease of disease symptoms. Other assays will be known to one of ordinary skill in the art and can be employed for measuring the level of the response.

The doses of cancer associated antigen compositions (e.g., polypeptide, peptide, antibody, cell or nucleic acid) administered to a subject can be chosen in accordance with different parameters, in particular in accordance with the mode of administration used and the state of the subject. Other factors include the desired period of treatment. In the event that a response in a subject is insufficient at the initial doses applied, higher doses (or effectively higher doses by a different, more localized delivery route) may be employed to the extent that patient tolerance permits.

In general, for treatments for eliciting or increasing an immune response, doses of cancer associated antigen are formulated and administered in doses between 1 ng and 1 mg, and preferably between 10 ng and 100 μg, according to any standard procedure in the art. Where nucleic acids encoding cancer associated antigen of variants thereof are employed, doses of between 1 ng and 0.1 mg generally will be formulated and administered according to standard procedures. Other protocols for the administration of cancer associated antigen compositions will be known to one of ordinary skill in the art, in which the dose amount, schedule of injections, sites of injections, mode of administration (e.g., intra-tumoral) and the like vary from the foregoing. Administration of cancer associated antigen compositions to mammals other than humans, e.g. for testing purposes or veterinary therapeutic purposes, is carried out under substantially the same conditions as described above.

Where cancer associated antigen peptides are used for vaccination, modes of administration which effectively deliver the cancer associated antigen and adjuvant, such that an immune response to the antigen is increased, can be used. For administration of a cancer associated antigen peptide in adjuvant, preferred methods include intradermal, intravenous, intramuscular and subcutaneous administration. Although these are preferred embodiments, the invention is not limited by the particular modes of administration disclosed herein. Standard references in the art (e.g., Remington's Pharmaceutical Sciences, 18th edition, 1990) provide modes of administration and formulations for delivery of immunogens with adjuvant or in a non-adjuvant carrier.

When administered, the pharmaceutical preparations of the invention are applied in pharmaceutically-acceptable amounts and in pharmaceutically-acceptable compositions. The term “pharmaceutically acceptable” means a non-toxic material that does not interfere with the effectiveness of the biological activity of the active ingredients. Such preparations may routinely contain salts, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

A cancer associated antigen composition may be combined, if desired, with a pharmaceutically-acceptable carrier. The term “pharmaceutically-acceptable carrier” as used herein means one or more compatible solid or liquid fillers, diluents or encapsulating substances which are suitable for administration into a human. The term “carrier” denotes an organic or inorganic ingredient, natural or synthetic, with which the active ingredient is combined to facilitate the application. The components of the pharmaceutical compositions also are capable of being co-mingled with the molecules of the present invention, and with each other, in a manner such that there is no interaction which would substantially impair the desired pharmaceutical efficacy.

The pharmaceutical compositions may contain suitable buffering agents, including: acetic acid in a salt; citric acid in a salt; boric acid in a salt; and phosphoric acid in a salt.

The pharmaceutical compositions also may contain, optionally, suitable preservatives, such as: benzalkonium chloride; chlorobutanol; parabens and thimerosal. The pharmaceutical compositions may conveniently be presented in unit dosage form and may be prepared by any of the methods well-known in the art of pharmacy. All methods include the step of bringing the active agent into association with a carrier which constitutes one or more accessory ingredients. In general, the compositions are prepared by uniformly and intimately bringing the active compound into association with a liquid carrier, a finely divided solid carrier, or both, and then, if necessary, shaping the product.

Compositions suitable for oral administration may be presented as discrete units, such as capsules, tablets, lozenges, each containing a predetermined amount of the active compound. Other compositions include suspensions in aqueous liquids or non-aqueous liquids such as a syrup, elixir or an emulsion.

Compositions suitable for parenteral administration conveniently comprise a sterile aqueous or non-aqueous preparation of cancer associated antigen polypeptides or nucleic acids, which is preferably isotonic with the blood of the recipient. This preparation may be formulated according to known methods using suitable dispersing or wetting agents and suspending agents. The sterile injectable preparation also may be a sterile injectable solution or suspension in a non-toxic parenterally-acceptable diluent or solvent, for example, as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono-or di-glycerides. In addition, fatty acids such as oleic acid may be used in the preparation of injectables. Carrier formulation suitable for oral, subcutaneous, intravenous, intramuscular, etc. administrations can be found in Remington's Pharmaceutical Sciences, Mack Publishing Co., Easton, Pa.

### EXAMPLES

### Example 1

### Identification of Mouse Tumor Antigens by SEREX

To investigate tumor specificity in host immune reactions to SEREX antigens, the well-controlled mouse tumor model BALB/c methylcholanthrene-induced fibrosarcoma Meth A was used. Meth A is a prototype tumor for analysis of tumor rejection antigens (Gross, L., Cancer Res. 3:326-333, 1943; Foley, Cancer Res. 13:835-842, 1953; Prehn et al., J. Natl. Cancer. Inst. 18:769-778, 1957; and Old et al., Ann. N.Y. Acad. Sci. 101:80-106, 1962).

Materials and Methods

Mice

BALB/c mice were purchased from Japan SLC (Shizuoka, Japan).

Tumors and Sera.

Meth A, CMS5a, CMS5j, CMS8, CMS 13 and CMS 17 are methylcholanthrene-induced fibrosarcomas in BALB/c mice (DeLeo et al., J. Exp. Med. 146:720-734, 1977; Palladino et al., Cancer Res. 47:5074-5079, 1987). RL♂ 1 is a radiation-induced leukemia in a BALB/c mouse (Nakayama et al., Proc. Natl. Acad. Sci USA 76:3486-3490, 1979). RVA, RVC and RVD are leukemias induced by an injection of radiation leukemia virus into neonatal BALB/c mice (Stockert et al., J. Exp. Med. 149:200-215, 1984). RV2 is leukemia induced by an injection of radiation leukemia virus into a neonatal C57BL/6 mouse (Nakayama et al., Cancer Res. 44:5138-5144, 1984). A20.2J is a BALB/c B cell lymphoma (Kim et al., J. Immunol. 122:549-554, 1979). MOPC-70A is a mineral oil-induced myeloma in a BALB/c mouse (Potter, M. In: Methods in Cancer Research, 1967, pp. 106-157). P815 is a methylcholanthrene-induced mastocytoma in a DBA/2 mouse (Dunn et al., J. Natl. Cancer Inst. 18:587-601, 1957). EL4 is an dimethylbenzanthracene-induced leukemia in a C57BL mouse (Gorer et al., Br. J. Cancer 4:372-379, 1950).

Meth A (Kγ) cells used for construction of cDNA expression library were Meth A cells which were retrovirally introduced with murine IFN-γ cDNA (Watanabe et al., Eur. J. Immunol. 18:1627-1630, 1988). Meth A (Kγ) cells were highly antigenic and regressed in CB6F₁ mice. Concomitant inoculation of the cells caused rejection of the parental Meth A cells (Harutsumi et al., Int. J. Oncol. 7:233-238, 1995).

Sera were obtained from BALB/c mice at various days after 5×10⁵ Meth A inoculation (i.d. in the back). Sera were obtained from 3-5 mice each at 16, 23, 31 and 45 days after MethA inoculation.

Construction of cDNA Expression Library.

Poly (A)⁺ RNA was purified from Meth A (Kγ) cells and a cDNA expression library was constructed in a λZAP Express vector using a cDNA library kit (Stratagene, La Jolla, Calif.).

Immunoscreening of cDNA Library.

cDNA libraries was screened with serum from BALB/c mice obtained on day 45 after inoculation of Meth A. The nitrocellulose membranes containing the phages were incubated overnight at room temperature with the serum diluted to 1:200, which has been preabsorbed with lysate from E. coli coupled to Sepharose 4B (5 Prime→3 Prime, Boulder, Colo.). Serum antibodies binding to recombinant proteins expressed in lytic phages were detected by incubation with peroxidase-conjugated goat anti-mouse IgG (Jackson Immuno Research, West Grove, Pa.) and visualized by staining with 3-3′-diaminobenzidine (Sigma, St. Louis, M@).

Sequence Analysis of Reactive Clones.

The reactive clones were subcloned to monoclonality and excised in vivo to pBK-CMV plasmid forms (Stratagene, La Jolla, Calif.). The nucleotide sequence of cDNA inserts was determined by using ABI PRISM automated sequencers (PE Applied Biosystems, Foster City, Calif.). Sequence alignments were performed with BLAST software on GenBank.

Analysis of Antigenicity of SEREX Antigens.

To analyze antigenicity of SEREX antigens, sera from BALB/c mice bearing CMS5a, CMS5j, CMS8, CMS13 and CMS 17 were used for the detection of antibodies with the immunoscreening assay described above. Serum from BALB/c mice obtained 42 weeks s.c injection with 3-methylcholanthrene dissolved in peanut oil at a concentration of 0.125 μg/ml was also used.

Reverse Transcription PCR (RT-PCR).

mRNA was purified from normal BALB/c mouse tissues and tumors using the QuickPrep Micro mRNA Purification Kit (Amersham Pharmacia, Piscataway, N.J.). The mRNA was reverse transcribed into single strand cDNA using Moloney murine leukemia virus reverse transcriptase and oligo(DT)₁₅ as primer (Amersham Pharmacia, Piscataway, N.J.). cDNAs were tested for integrity by amplification of β-actin transcripts in a 30 cycle reaction. Gene specific oligonucleotide primers were designed to amplify cDNA segments for 300-600 bp in length. RT-PCR was performed by using 30 amplification cycles at an annealing temperature of 58° C. using the following primers:

| | |
|---|---|
| MC4A 5′-GTGGACAAGAGGAAGCACAA-3′ | (SEQ ID NO:21) |
| and | |
| MC4B 5′-TGAAAAGTAAGGGCTGTCAT-3′. | (SEQ ID NO:22) |

Isolation and Characterization of Antigens in Meth A by SEREX.

cDNA expression libraries were prepared from Meth A (Kγ) cells, which were Meth A cells retrovirally introduced with IFN-γ in order to augment antigenicity. Immunoscreening of 80,000 clones with serum from BALB/c mice obtained at day 45 after inoculation of parental Meth A yielded a total of 35 positive clones. The nucleotide sequences of the cDNA inserts were determined. As shown in Table 1, ten different genes were isolated (OY-MC-1 through OY-MC-10), five of which were identical to or had homology to genes in the DNA database. Of these, OY-MC-1 was represented by 13 overlapping clones, OY-MC-2 by 10 overlapping clones, OY-MC-3 by 3 overlapping clones, OY-MC-4 by 2 overlapping clones and OY-MC-6 by a single clone. OY-MC-1 was homologous to rat ribosomal protein L11. OY-MC-3 was homologous to bovine NAD⁺ dependent isocitrate dehydrogenase subunits 3 and 4. OY-MC-2 was homologous to mouse glyceraldehyde-3-phosphate dehydrogenase (GAPDH). OY-MC-4 was homologous to mouse placenta and embryonic expression gene (pem). OY-MC-6 was identical to a C4 and cytochrome P450 hydroxylase A gene. The remaining five genes (OY-MC-5, 7, 8, 9, 10) were unknown genes which had no homology to any published genes.

TABLE 1
Genes Isolated from Meth A by SEREX

| Gene | Size, bp | SEQ ID NO | Homology/Identity | Frequency |
|---|---|---|---|---|
| OY-MC-1 | 585 | 1 | R. rattus ribosomal | 13/35 |
| | | | protein L11 | |
| OY-MC-2 | 1228 | 3 | mouse glyceraldehyde- | 10/35 |
| | | | 3-phosphate | |
| | | | dehydrogenase | |
| OY-MC-3 | 1758 | 5 | B. taurus NAD⁺ | 3/35 |
| | | | dependent isocitrate | |
| | | | Dehydrogenase | |
| | | | subunits 3 and 4 | |
| OY-MC-4 | 845 | 7 | mouse placenta and | 2/35 |
| | | | embryonic expression | |
| | | | gene (pem) | |
| OY-MC-5 | ˜1,000 | 9 | No strong homology | 2/35 |
| OY-MC-6 | ˜2,100 | 11 | C4 and cytochrome | 1/35 |
| | | | P450 hydroxylase A | |
| OY-MC-7 | ˜1,400 | 13 | No strong homology | 1/35 |
| OY-MC-8 | 612 | 15 | No strong homology | 1/35 |
| OY-MC-9 | 1,036 | 17 | No strong homology | 1/35 |
| OY-MC-10 | ˜500 | 19 | No strong homology | 1/35 |

Expression of SEREX-Defined Antigens in Normal Tissues and Tumors.

mRNA expression for the genes were examined by RT-PCR, using a panel of normal mouse tissues and tumors. OY-MC-1, OY-MC-2 and OY-MC-9 showed ubiquitous mRNA expression in normal tissues, other genes showed a variety of expression patterns. Of those, OY-MC-4 showed a restricted expression in normal tissues as it was expressed only in testis and placenta, and to a lesser extent in ovary (Table 2).

TABLE 2
mRNA Expression of OY-MC-4 in Normal Mouse Tissues

| Tissue | MRNA | Tissue | MRNA |
|---|---|---|---|
| Brain | − | Kidney | − |
| Fetal brain | − | Pancreas | − |
| Salivary gland | − | Stomach | − |
| Esophagus | − | Duodenum | − |
| Thymus | − | Small intestine | − |
| Heart | − | Colon | − |
| Lung | − | Bladder | − |
| Liver | − | Uterus | − |
| Fetal liver | − | Ovary | +^{#} |
| Spleen | − | Testis | + |
| Lymph node | − | Placenta | + |
| Adrenal gland | − | Bone marrow | − |

^{#}Weak expression

Nine out of ten genes except OY-MC4 showed ubiquitous expression in 15 mouse tumors tested. Expression of OY-MC4 was rather restricted and found in 10 of 15 mouse tumors tested (Table 3). Of 6 methylcholanthrene-induced fibrosarcomas examined, Meth A, CMS5j, CMS8 and CMS13 showed OY-MC-4 expression, CMS17 showed a faint expression, and CMS5a was negative. Expression of OY-MC4 was much less frequent in 6 leukemias examined. Strong expression of OY-MC-4 was only found in E14. RVD and RV2 showed a faint expression, and RL♂ 1, RVA, RVC were negative (Table 3).

TABLE 3
mRNA Expression of OY-MC-4 in Mouse Tumors

| Tumor | MRNA | Tumor | MRNA |
|---|---|---|---|
| Meth A | + | RVC | − |
| CMS5a | − | RVD | ±^{#} |
| CMS5j | + | RV2 | ±^{#} |
| CMS8 | + | EL4 | + |
| CMS13 | + | A20.2j | + |
| CMS17 | ±^{#} | MOPC-70A | − |
| RL♂ 1 | − | P815 | + |
| RVA | − | | |

^{#}Faint expression.

cDNA and Protein Sequence of OY-MC-4.

The nucleotide sequence of OY-MC-4 revealed that the cDNA was 845 bp in length (SEQ ID NO:7) and nearly identical to a mouse homeobox gene, placenta and embryonic expression gene (pem). The pem gene was identified from an AKR T-cell lymphoma and shown to be coded for by the X chromosome (Wilkinson et al., Dev. Biol. 141:451-455, 1990; Lin et al., Dev. Biol. 166:170-179, 1994). One nucleotide difference (C to A) at position 619 was observed in OY-MC4 compared with the published pem sequence. This nucleotide difference results in a Thr→Asp change at amino acid residue 174 (SEQ ID NO:8) as compared with the published pem sequence. The sequences of OY-MC4 PCR products from Meth A, CMS5j, CMS8, CMS13 and testis that were subcloned into pCR2.1 showed A (adenine) at position 619, indicating that A at position 619 of SEQ ID NO:7 is a normal pem nucleotide sequence in BALB/c mouse.

Reactivity of Sera of Various Stages of Meth A Bearing Mice.

To determine the occurrence of antibodies against the defined antigens, sera obtained at various stages from parental Meth A bearing BALB/c mice were tested. As shown in Table 4, antibodies against 7 out of 10 antigens were detected in sera from mice at day 31 after inoculation of Meth A, and increased in titer at day 45. OY-MC-4, OY-MC-6 and OY-MC-8 were only detected by sera obtained on day 45.

TABLE 4
Reaction of Serum Obtained From Various
Stages of Meth A Bearing Mice

Days After Tumor Inoculation

| Gene | 16d | 23d | 31d | 45d |
|---|---|---|---|---|
| OY-MC-1 | − | − | + | + |
| OY-MC-2 | − | ± | + | + |
| OY-MC-3 | − | + | + | + |
| OY-MC-4 | − | − | ± | + |
| OY-MC-5 | − | − | + | + |
| OY-MC-6 | − | − | ± | + |
| OY-MC-7 | − | − | + | + |
| OY-MC-8 | − | − | − | + |
| OY-MC-9 | − | − | + | + |
| OY-MC-10 | − | − | + | + |

Sera obtained from mice at day 45 after Meth A inoculation showed the strongest reactivities against all of the antigens compares with those from mice at day 31 and 23 after Meth A inoculation.

Antigenicity of SEREX Defined-Antigens.

To analyze antigenicity of SEREX-defined antigens, sera from BALB/c mice bearing a panel of methylcholanthrene sarcomas were studied. As shown in Table 5, all the antigens were only detected with sera from the parental Meth A bearing mice.

TABLE 5
Specificity of SEREX antigens

| | Tumor | | | | | |
|---|---|---|---|---|---|---|
| | Meth A | CMS 5a# | CMS 5j# | CMS 8# | CMS 13# | Methylcholanthrene* |
| OY-MC-1 | + | − | − | − | − | − |
| OY-MC-2 | + | − | − | − | − | − |
| OY-MC-3 | + | − | − | − | − | − |
| OY-MC-4 | + | − | − | − | − | − |
| OY-MC-5 | + | − | − | − | − | − |
| OY-MC-6 | + | − | − | − | − | − |
| OY-MC-7 | + | − | − | − | − | − |
| OY-MC-8 | + | − | − | − | − | − |
| OY-MC-9 | + | − | − | − | − | − |
| OY-MC-10 | + | − | − | − | − | − |

# Methylcholanthrene-induced fibrosarcomas.
*BALB/c mice were injected s. c. with 3-methylcholanthrene dissolved in peanut oil at a concentration of 125 μg/ml in a volume of 0.2 ml. Serum was obtained 42 weeks after injection.

Ten different genes were identified by immunoscreening of a cDNA expression library from Meth A (Kγ) cells with sera from parental Meth A bearing BALB/c mice. Of those 10 genes, 5 were identical to or had homology to genes in the DNA database, and 5 were unknown genes. RT-PCR analysis revealed a variety of mRNA expression patterns of the genes in normal mouse tissues. Of most interest was OY-MC-4, which showed a restricted expression in normal tissues. It was strongly expressed in testis and placenta, and to a lesser extent in ovary. No expression was found in the other 21 out of 24 normal tissues tested. Furthermore, OY-MC-4 was selectively expressed in a variety of mouse tumors. Of 6 methylcholanthrene-induced fibrosarcomas examined, 4 (Meth A, CMS5j, CMS8, CMS13) showed OY-MC-4 expression, one (CMS17) showed a faint expression, whereas CMS5a was negative. OY-MC-4 expression in leukemias was much less frequent than those in fibrosarcomas: only EL4 showed strong expression, other five were negative or showed a faint expression.

Nucleotide sequencing analysis revealed that OY-MC-4 was nearly identical to mouse placenta and embryonic expression gene (pem), which was previously shown to be expressed during embryogenesis (Wilkinson et al., Dev. Biol. 141:451-455, 1990). Pem gene was distantly related to theprd/pax homeobox gene family (Lin et al., Dev. Biol. 166:170-179, 1994; Sasaki et al., Mech. Dev. 34:155-164, 1991) and was shown to be coded for by the X chromosome (Lin et al., Dev. Biol. 166:170-179, 1994; Maiti et al., Genomics 34:304-316, 1996). OY-MC-4 nucleotide sequence (SEQ ID NO:7) differed from the published pem cDNA sequence, which was isolated from a T-lymphoma of AKR mouse, in having an A instead of a C at position 619. The corresponding predicted amino acid sequence of OY-MC-4 (SEQ ID NO:8) included asparagine instead of threonine at position 174. Threonine at position 174 was thought to be a phosphorylation site for protein kinase C (Wilkinson et al., 1990. Dev. Biol. 141:451455). OY-MC-4 cDNAs isolated from other tumors (CMS5j, CMS8, CMS13) and normal BALB/c testis had A at position 619, indicating that the sequence isolated from Meth A is not a point mutation but a normal nucleotide sequence of pem in BALB/c mouse. Taken together, the findings suggeested that OY-MC4 is a new cancer/testis (CT) antigen isolated by the SEREX method.

There are a growing number of genes belonging to the CT antigen family, including MAGE, BAGE, GAGE, HOM-MEL-40/SSX2, NY-ESO-1, SCP1 and CT7. Of those, MAGE and NY-ESO-1 have been shown to elicit both humoral and cytotoxic T lymphocyte responses. NY-ESO-1 was isolated from an esophageal squamous cell carcinoma. NY-ESO-1 has been shown to be recognized by CTL, in the context of HLA-A2 and HLA-A31. SCP1, which was isolated from a cDNA library enriched for testis specific clones by using sera from patients with renal cancer, is presently the only CT antigen with known function. The SCP1 protein is involved in the meiotic chromosome synapses of sperm cells. OY-MC-4, described here, is a new member of SEREX-defined CT antigens with known function.

### Example 2

### Identification of Acrosomal Protein, sp32, as a New Human Cancer/Testis Antigen

RNA isolation and cDNA synthesis.

Total RNA was isolated from human testis using the RNeasy kit (Qiagen GmbH, Germany). Poly (A)⁺ RNA was isolated from tumor tissue using the QuickPrep Micro mRNA Purification kit (Amersham Pharmacia, Piscataway, N.J.). The mRNA was reverse transcribed into single strand cDNA using the Moloney murine leukemia virus reverse transcriptase and oligo(dT)₁₅ as a primer (Amersham Pharmacia, Piscataway, N.J.).

PCR Amplification and EST Cloning.

Primers used in this study are listed below in Table 6:

TABLE 6
Amplification and Cloning Primers

1. For 1st round PCR amplification

| | | |
|---|---|---|
| Pem 5 | (SEQ ID NO:25) | 5′-GTGGACAAGAGGAAGCACAA-3′ |
| | | (derived from OY-MS-4) |
| EST-2 | (SEQ ID NO:26) | 5′-TCTCCCCATCTCACTCCAC-3′ |
| | | (derived from human testis EST clone, zt86b04.r1 |
| | | [GenBank accession number AA397852]) |

2. For 2nd and 3rd PCR amplification, and analysis of mRNA expression in
normal and malignant tissues

| | | |
|---|---|---|
| ht-5 | (SEQ ID NO:27) | 5′-AAGGACAGGGGACTAAGGAG-3′ |
| ht-3 | (SEQ ID NO:28) | 5′-CCGTACAAATCCAGCCCGTA-3′ |
| ht-1 | (SEQ ID NO:29) | 5′-ATGTGAGTAGGGGCCGAGTA-3′ |

3. For 5′RACE

| | | |
|---|---|---|
| GSP1-T1 | (SEQ ID NO:30) | 5′-TTCCTGGGCTGATCGAATGAG-3′ |
| GSP2-T1 | (SEQ ID NO:31) | 5′-GCAAAAGAGGAAGGGTTAGAAG-3′ |
| | | (nested primer) |
| Tes-N1 | (SEQ ID NO:32) | 5′-CCGTGGTTTTCATATTGGTC-3′ |
| | | (sequence primer) |

A search was initiated for a human homologue gene of OY-MC4 (pem), which had been found in murine sarcoma Meth A by SEREX analysis as described above. Human testis cDNA was amplified by PCR using primers pem5 (SEQ ID NO:25) from mouse OY-MC-4 and EST-2 (SEQ ID NO:26) from an EST database derived human testis clone. For first round PCR amplification, human testis cDNA was supplemented with 5 μof 10×PCR buffer (100 mM Tris-HCl, 500 mM KCl, 0.01% (w/v) gelatin), 3 μl of 25 mM MgCl₂, 2 μl of 10 mM-dNTPs, 5 μl of 5 μM each primer (pem5 and EST-2), 1.25 units of AmpliTaq DNA Polymerase (Perkin Elmer) and water to a final volume of 50 μl. PCR cycle conditions were 1 min at 94° C., 1 min at 54° C. and 1.5 min at 72° C. for 35 cycles. These cycles were preceded by 1 min of denaturation at 94° C. and followed by a 10 min elongation step at 72° C.

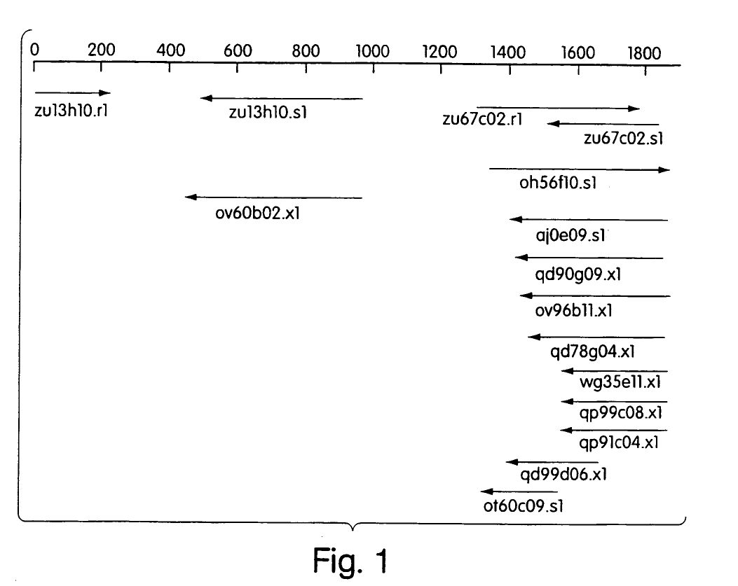

A PCR product of 1.1 kb was obtained and sequenced using an ABI PRISM automated sequencer. Homology walking was performed using the human dbEST database maintained by the National Center for Biotechnology Information (http:/www.ncbi.nlm.nih.gov/dbEST). Several ESTs corresponded to the PCR product. Most of the ESTs were cDNA clones derived from human testis. The EST clusters are shown in FIG. 1. The 3′ end of the gene was extended by the sequence information of more than 11 ESTs which exhibited homology to the PCR product. Nucleotide sequence (nucleotides 261-1796 of SEQ ID NO:23) of the PCR product of 1.5 kb which was obtained using only anti-sense primer ht-1 (SEQ ID NO:29) was identical to the deduced sequence.

5′-RACE amplification.

5′-RACE was performed to identify the cDNA 5′ end sequence using the 5′ RACE System for Rapid Amplification kit following the manufacturer's protocols (Life Technologies, Inc., Rockville, Md.). Total RNA from human testis was used as a template and first-strand cDNA was synthesized using specific primer, GSP1-T1 (SEQ ID NO:30). The RACE product was sequenced using an ABI PRISM automated sequencer with sequence primer, Tes-N1(SEQ ID NO:32).

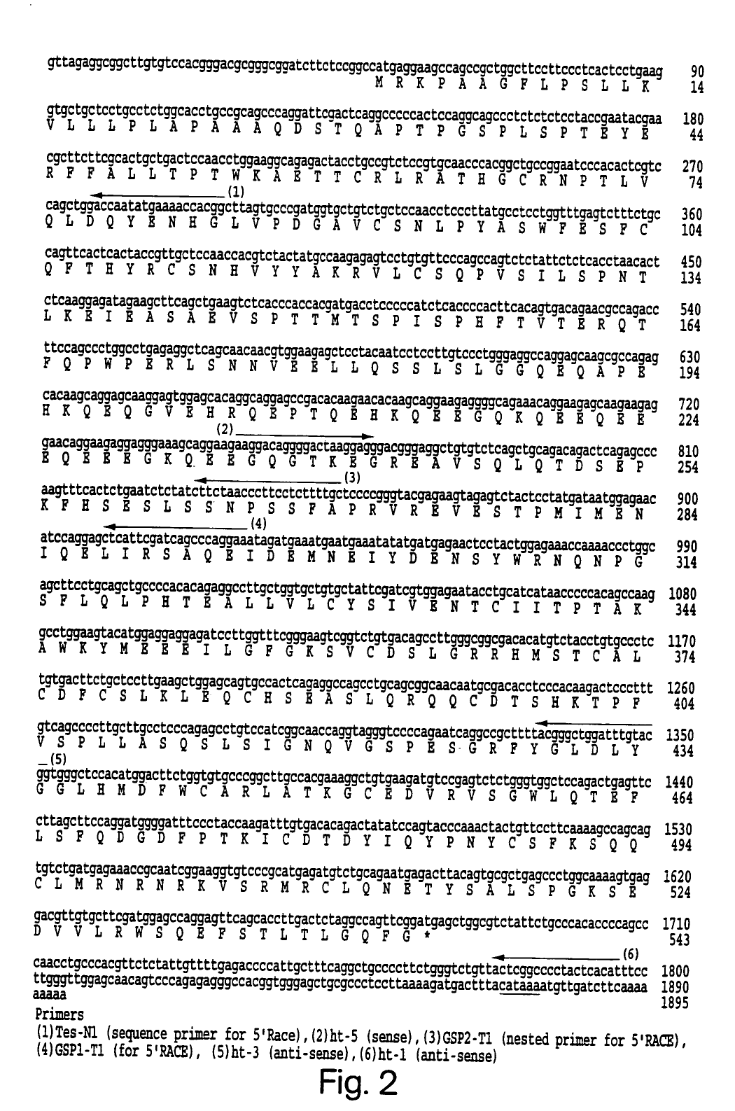

Full length cDNA nucleotide sequence (1895 bp, SEQ ID NO:23) and deduced amino acid sequence (SEQ ID NO:24) are shown in FIG. 2.

Homology.

A homology search of the cDNA sequence through the GenBank database revealed that the full length cDNA is the guinea pig and porcine acrosomal protein sp32 genes. Other homologous nucleic acid sequences are set forth in Table 11 below. The amino-terminal region composed of 25 residues in the protein was highly hydrophobic, suggesting that it was a signal sequence. The amino-terminal region composed of 24 residues in mature human sp32 corresponding to the same region composed of 25 residues in the porcine, 22 residues in the guinea pig, and 23 residues in the mouse are abundant in acidic amino acids.

Expression Profile of OY-TES-1.

Specific primers, ht-5 (SEQ ID NO:27) and ht-3 (SEQ ID NO:28), were designed to amplify cDNA segments from normal tissue (MTC panels; Clontech, Palo Alto, Calif.) and tumor samples. RT-PCR was performed as described above for 30 amplification cycles with an annealing temperature of 62° C. Expression of the mRNA was observed with only testis in normal adult tissues and with various malignant tumors (Table 7).

TABLE 7
OY-TES-1 mRNA expression in human tumors

| Tumor type | mRNA, positive/total |
|---|---|
| Bladder cancer | 11/39 |
| Lung cancer | 4/11 |
| Breast cancer | 2/5 |
| Hepatoma | 2/5 |
| Colon cancer | 2/13 |
| Stomach cancer | 0/5 |
| Renal cancer | 0/10 |

These findings indicated that the cDNA identified is the human homologue of acrosomal protein sp32 and has the expression characteristics of a cancer/testis antigen. Therefore, the cDNA was named OY-TES-1 antigen.

Radiation Hybrid Mapping.

PCR was applied to the Stanford G3 Radiation Hybrid Panel (purchased from Research Genetics, USA). PCR primers designed from the sequence of the 3′UTR of OY-TES-1 were as follows: sense, 5′-CTGGCGTCTATTCTGCCCA-3′ (SEQ ID NO: 33); antisense, 5′-TGTAAAGTCATCTITTAAGGAGG-3′ (SEQ ID NO: 34). PCR was performed under conditions of 33 cycles at 94° C. for 30 sec for denaturation at 57° C. for 30 sec for annealing, and at 72° C. for 30 sec for extension in a mixture containing 250 mM of each dNTP, 10 mM of each primer, PCR buffer (10 mM Tris-HCl, 50 mM KCl, 1.5 mM MgCl₂, pH 8.3), 0.5 unit of Taq polymerase (Takara Shuzo, Japan) and 25 ng of template DNA. The PCR products were loaded onto 6% polyacrylamide gels. The presence of specific PCR products was scored, and screening results were submitted to Online RH-server at Stanford Human Genome Center.

Isolation of PAC Clones Corresponding to OY-TES-1.

To obtain genomic clones for OY-TES-1, P1-derived artificial chromosomes (PACs) were isolated from a PAC library (Matsumoto, N. et al., Genomics 45:11-16, 1997) by a PCR screening using the primer set described above.

FISH Analysis.

PAC DNA probe was prepared by nick-translation with SpectrumGreen-dUTP (Vysis, Downers Grove, Ill.) and hybridized to R-banded metaphase chromosomes (Takahashi, E., et al., Human Genet. 86:14-16, 1990), with the use of D12Z3 DNA probe (Oncor, Gaithersburg, Md.) as reference. After chromosomes were counter-stained with DAPI, their fluorescence image was captured using a monochrome CCD camera (Carl Zeiss, Oberkochen, Germany) on an Axioplan fluorescence microscope (Carl Zeiss, Oberkochen, Germany) with appropriate filters. Multi-color fluorescence signals were merged with counter-staining images using ISIS2 software (Metasystems, Altlussheim, Germany).

Southern Blot Hybridization.

Genomic DNA was extracted from testis using DNeasy (Qiagen GmbH, Hilden, Germany). Genomic DNA was digested with 100 units of EcoRI, HindIII and BamHI at 37° C. overnight. The DNA was separated on an 0.8% agarose gel, blotted onto nylon transfer membrane (Hybond-N+, Amersham Pharmacia Biotech, Amersham Place, UK). The blot was hybridized to the 602-bp cDNA probe (nt 749-1351 of SEQ ID NO:23) directly labeled with alkaline phosphatase (AlkPhos Direct, Amersham. Pharmacia Biotech, Amersham Place, UK), washed and processed for chemiluminescence according to the manufacturer's instructions.

Recombinant OY-TES-1 Protein.

OY-TES-1 was expressed in E. coli using histidine-tag-containing vector pQE32 (Qiagen, Hilden, Germany). cDNA amplification primers were designed to encompass entire coding sequences of the gene, corresponding to amino acid positions from 1 to 543. The induction of recombinant protein synthesis and subsequent purification by Ni²⁺-NTA column were performed according to the manufacturer's instructions.

ELISA.

Recombinant OY-TES-1 protein (2 μg/ml) in 0.05M carbonate buffer (pH 9.6) was absorbed to 96 well plates (Nunc, Rostilde, Denmark) at 4° C. overnight. Plates were washed with PBS/Tween and blocked with 5% FCS/PBS at room temperature for 1 hr. After washing, serum dilutions (100 μl) in 5% FCS/PBS was added and incubated at room temperature for 2 hr. Plates were washed and incubated with secondary antibody (HRP-conjugated goat-anti human IgG, Medical Biological Lab., Tokyo, Japan) at 1/2000 dilution for 1 hr at room temperature. Plates were washed and incubated with substrate solution (1,2-phenylenediamine dihydrochloride) for 20 min at room temperature. After addition of 3M H₂SO₄ (100 μl), the absorbance was determined with a microplate reader (Toso Ltd., Tokyo, Japan).

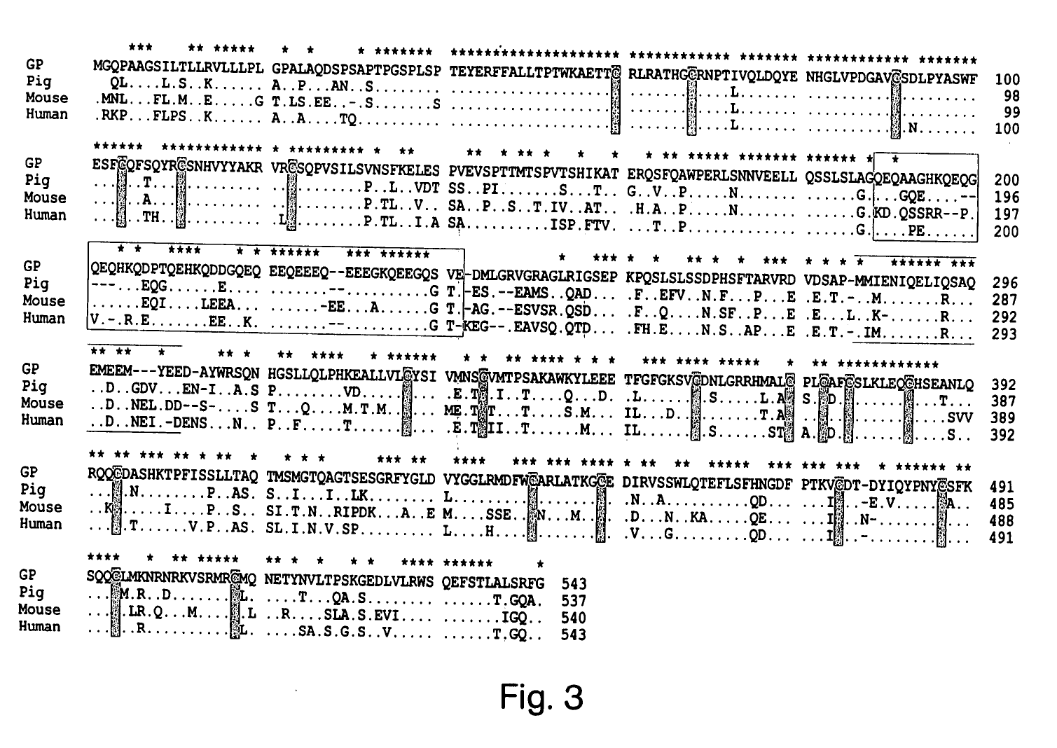

5′ RACE amplification was further performed to obtain full-length cDNA. The cDNA obtained, designated as OY-TES-1, is 1895 bp in length (SEQ ID NO:23). Part of the sequences in the pem5 and EST-2 primers were found in OY-TES-1: 5′-CCCCATCTCACCCCAC-3′ (SEQ ID NO: 35) from EST-2 corresponding to nt 501-516 of SEQ ID NO:23 with one nucleotide (underlined) difference and 3′-CTTCGTGTT-5′ (SEQ ID NO: 36) from pem5 corresponding to nt 1633-1625 of SEQ ID NO:23. OY-TES-1 contains a single long open reading frame that extends from bp 49 to 1677 and predicts a protein containing 543 amino acids (SEQ ID NO:24; FIG. 2). A homology search of the gene through the GenBank database revealed that it is a human homologue of the gene coding for porcine, guinea pig and mouse proacrosin binding protein sp32 (Baba, T., et al., J. Biochem. 269:10133-10140,1994), but showed no homology to mouse pem gene. The deduced amino acid sequence of OY-TES-1 showed a high degree of identity with the porcine (81.9%), the guinea pig (77.2%) and the mouse (75.2%) sequence (FIG. 3). It contained a glutamic acid- and glutamine rich-domain (EQ-rich domain) and acidic amino acid regions. Twenty cysteine residues were also totally conserved.

Southern Blot Analysis of OY-TES-1.

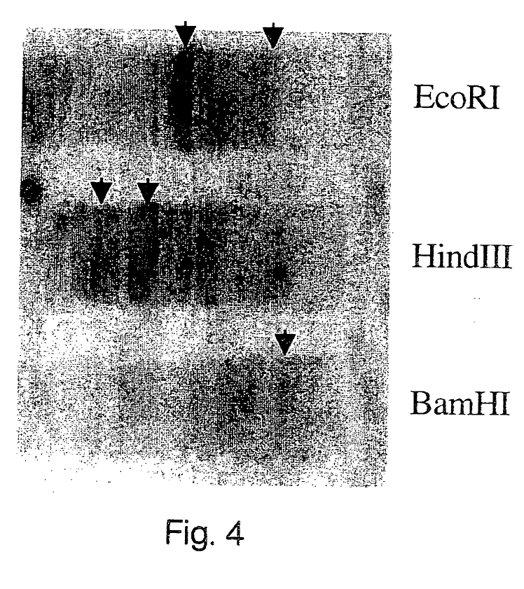

Genomic Southern blot analysis on testis and a renal cell carcinoma with OY-TES-1 probe showed two bands in EcoRI and HindIII digests, suggesting that there are two homologue genes in human genome (FIG. 4).

Chromosomal Assignment of OY-TES-1.

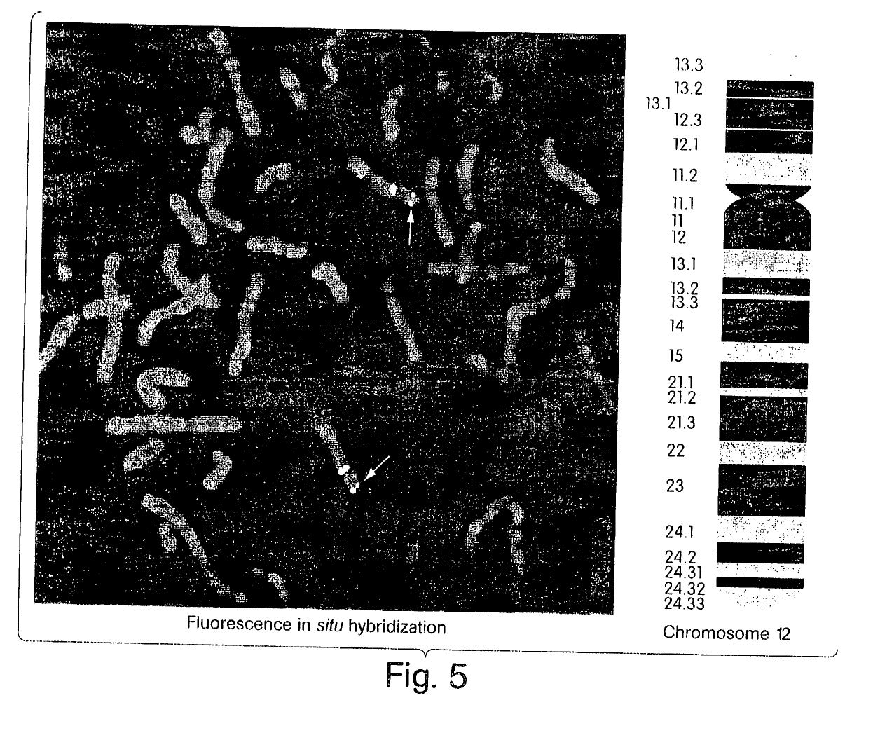

The scores obtained with the radiation hybrid panel were as follows: 00100-01001-01000-01001-01000-10000-00000-00010-00110-01100-0001′-00001-01000-00110-00000-01000-000. According to the online RH-server, the result was matched to the cell line SHGC-12737 that was mapped to chromosome 12 and a reference interval estimated was 13.9 cM between two markers, D12S99-D12S358. More fine chromosomal localization was defined by the FISH analysis with a PAC probe (145N7): of the 20 metaphase cells analyzed, 18 showed brightly fluorescent twin signals 12p12-p13 (FIG. 5).

Genomic Structure of OY-TES-1.

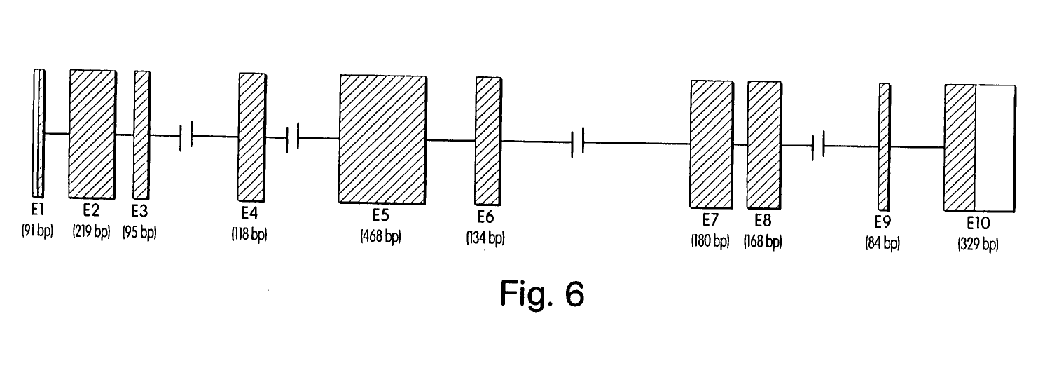

Genomic sequence of OY-TES-1 was searched in the GenBank hgts database by running the publicly available BLASTN software. OY-TES-1 cDNA sequence was found in the sequences of PAC clone RP4-761J14 and BAC clone RP11-433J6. Those two genes are located on chromosome 12p13, confirming the FISH analysis. As shown FIG. 6, the OY-TES-1 gene is composed of 10 exons, spanning a distance of 9339 bp.

Expression Pattern of OY-TES-1 in Normal and Malignant Tissues.

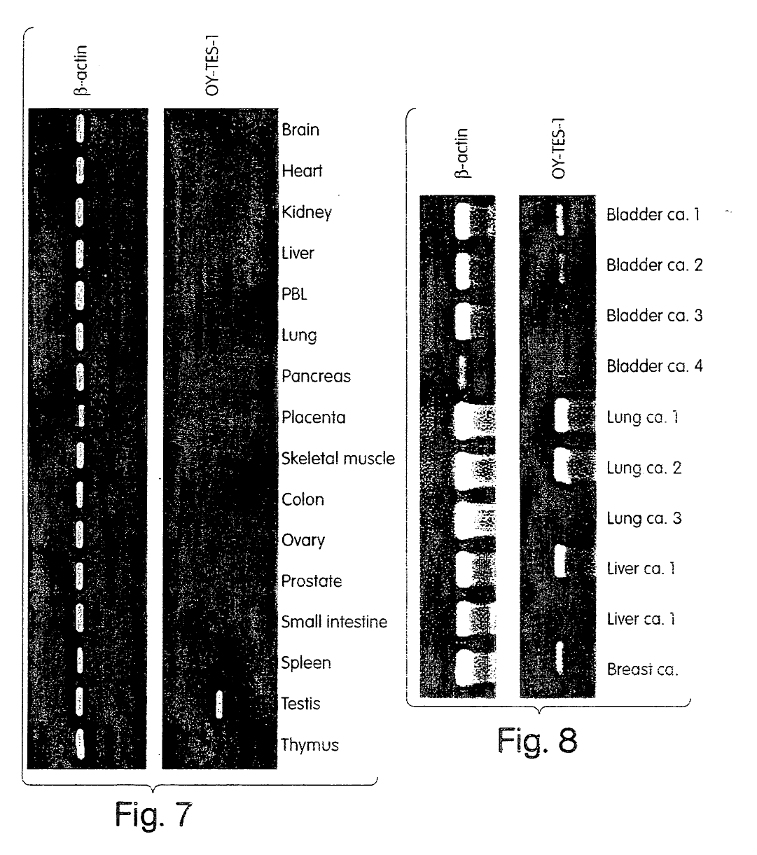

OY-TES-1 mRNA expression was examined by RT-PCR using a panel of normal and malignant tissues. As shown in FIG. 7, in adult tissues, OY-TES-1 expression was observed only in testis. In tumors, OY-TES-1 was expressed at various frequencies in different origin. As shown in FIG. 8 and Table 7, 28% (11/39) of bladder cancer tumor samples, 36% (4/11) of lung cancer tumor samples, 40% (2/5) of breast cancer tumor samples, 40% (2/5) of liver cancer tumor samples and 15% (2/13) of colon cancer tumor samples showed detectable OY-TES-1 mRNA. However, no expression of OY-TES-1 mRNA was observed in renal or stomach cancer tumor samples.

Seroreactivity Against OT-TES-1 in Cancer Patients.

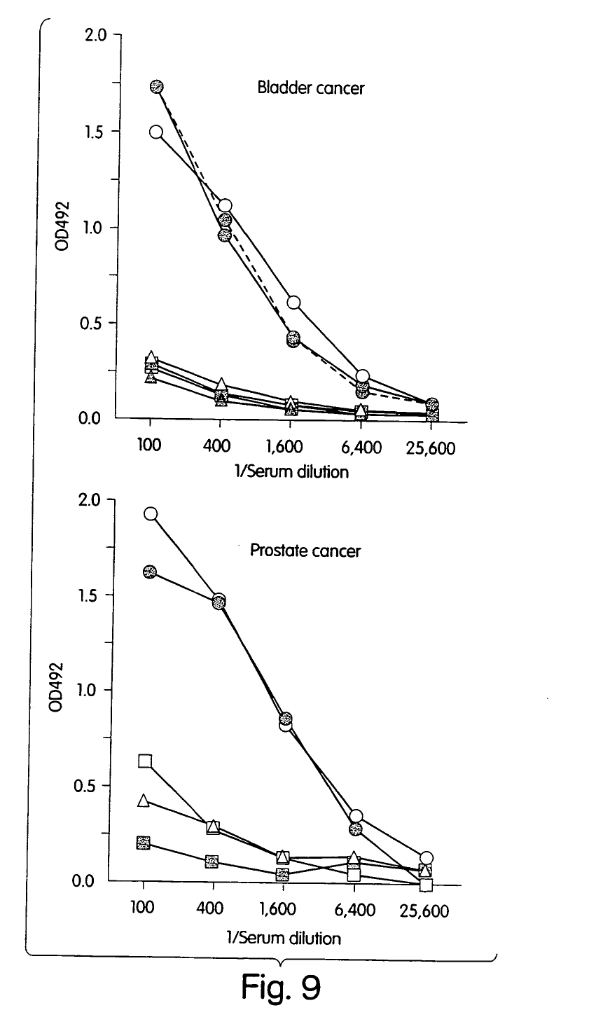

Antibody production against OY-TES-1 in sera from 334 cancer patients and 20 normal individuals was tested by ELISA using recombinant OY-TES-1 protein. As shown in Table 8, 5.3% (3/58) of bladder cancer patients, 3.4% (2/57) of prostate cancer patients, 10.5% (10/95) of liver cancer patients, 10.3% (6/58) of colon cancer patients and 4.5% (3/66) of lung cancer patients had antibodies against OY-TES-1 protein. None of the normal individuals had serum antibodies against OY-TES-1 protein. FIG. 9 illustrates titration curves with sera from selected bladder and prostate cancer patients.

TABLE 8
ELISA reactivity of sera from normal blood donors and
cancer patients with OY-TES-1

| Sera | ELISA, positive/total | |
|---|---|---|
| Healthy donors | 0/20 | (0%) |
| Bladder cancer | 3/58 | (5.3%) |
| Prostate cancer | 2/57 | (3.4%) |
| Liver cancer | 10/95 | (10.5%) |
| Colon cancer | 6/58 | (10.3%) |
| Lung cancer | 3/66 | (4.5%) |
| Total | 24/334 | (7.2%) |

In bladder cancer patients, both fresh-frozen specimens and serum samples were available from 13 patients. Tumors were typed for OY-TES-1 mRNA expression by RT-PCR and sera were assayed for OY-TES-1 antibody by ELISA. One out of 6 patients with OY-TES-1 mRNA positive tumors had OY-TES-1 antibody. No OY-TES-1 antibody was detected in sera from 7 patients with OY-TES-1 mRNA-negative tumors (Table 9).

TABLE 9
Correlation between OY-TES-1 mRNA expression and antibody
response in bladder cancer patients

| RT-PCR typing | ELISA | Number of cases |
|---|---|---|
| + | + | 1 |
| + | − | 5 |
| − | − | 7 |

### Example 3

### Preparation of Recombinant Cancer Associated Antigens

To facilitate screening of mice or patients' sera for antibodies reactive with cancer associated antigens, for example by ELISA, recombinant proteins are prepared according to standard procedures. In one method, the clones encoding cancer associated antigens are subcloned into a baculovirus expression vector, and the recombinant expression vectors are introduced into appropriate insect cells. Baculovirus/insect cloning systems are preferred because post-translational modifications are cared out in the insect cells. Another preferred eukaryotic system is the Drosophila Expression System from Invitrogen. Clones which express high amounts of the recombinant protein are selected and used to produce the recombinant proteins. The recombinant proteins are tested for antibody recognition using serum from the mouse or patient which was used to isolated the particular clone, or in the case of cancer associated antigens recognized by allogeneic sera, by the sera from any of the mouse strains or patients used to isolate the clones or sera which recognize the clones' gene products.

Alternatively, the cancer associated antigen clones are inserted into a prokaryotic expression vector for production of recombinant proteins in bacteria. Other systems, including yeast expression systems and mammalian cell culture systems also can be used.

### Example 4

### Preparation of Antibodies to Cancer Associated Antigens

The recombinant cancer associated antigens produced as in Example 2 above are used to generate polyclonal antisera and monoclonal antibodies according to standard procedures. The antisera and antibodies so produced are tested for correct recognition of the cancer associated antigens by using the antisera/antibodies in assays of cell extracts of mice or patients known to express the particular cancer associated antigen (e.g. an ELISA assay). These antibodies can be used for experimental purposes (e.g. localization of the cancer associated antigens, immunoprecipitations, Western blots, etc.) as well as diagnostic purposes (e.g., testing extracts of tissue biopsies, testing for the presence of cancer associated antigens).

### Example 5

### Expression of Cancer Associated Antigens in Cancers of Similar and Different Origin

The expression of one or more of the cancer associated antigens is tested in a range of tumor samples to determine which, if any, other malignancies should be diagnosed and/or treated by the methods described herein. Tumor cell lines and tumor samples are tested for cancer associated antigen expression, preferably by RT-PCR according to standard procedures. Northern blots also are used to test the expression of the cancer associated antigens. Antibody based assays, such as ELISA and western blot, also can be used to determine protein expression. A preferred method of testing expression of cancer associated antigens (in other cancers and in additional same type cancer patients or mice) is allogeneic serotyping using a modified SEREX protocol (as described above).

In all of the foregoing, extracts from the tumors of mice or patients who provided sera for the initial isolation of the cancer associated antigens are used as positive controls. The cells containing recombinant expression vectors described in the Examples above also can be used as positive controls.

The results generated from the foregoing experiments provide panels of multiple cancer associated nucleic acids and/or polypeptides for use in diagnostic (e.g. determining the existence of cancer, determining the prognosis of a patient undergoing therapy, etc.) and therapeutic methods (e.g., vaccine composition, etc.).

### Example 6

### HLA Typing of Patients Positive for Cancer Associated Antigen

To determine which HLA molecules present peptides derived from the cancer associated antigens, cells of the patients which express the cancer associated antigens are HLA typed (mice likewise can be typed for relevant MHC molecules). Peripheral blood lymphocytes are taken from the patient and typed for HLA class I or class II, as well as for the particular subtype of class I or class II. Tumor biopsy samples also can be used for typing. HLA typing can be carried out by any of the standard methods in the art of clinical immunology, such as by recognition by specific monoclonal antibodies, or by HLA allele-specific PCR (e.g. as described in WO97/31126).

### Example 7

### Characterization of Cancer Associated Antigen Peptides Presented by MHC Class I and Class II Molecules

Antigens which provoke an antibody response in a subject may also provoke a cell-mediated immune response. Cells process proteins into peptides for presentation on MHC class I or class II molecules on the cell surface for immune surveillance. Peptides presented by certain MHC/HLA molecules generally conform to motifs. These motifs are known in some cases, and can be used to screen the cancer associated antigens for the presence of potential class I and/or class II peptides. Summaries of class I and class II motifs have been published (e.g., Rammensee et al., Immunogenetics 41:178-228; 1995). Based on the results of experiments such as those described above, the HLA types which present the individual cancer associated antigens are known. Motifs of peptides presented by these HLA molecules thus are preferentially searched.

One also can search for class I and class II motifs using computer algorithms. For example, computer programs for predicting potential CTL epitopes based on known class I motifs has been described (see, e.g., Parker et al, J. Immunol. 152:163, 1994; D'Amaro et al., Human Immunol. 43:13-18,1995; Drijfhout et al., Human Immunol. 43:1-12, 1995). HLA binding predictions can conveniently be made using an algorithm available via the Internet on the National Institutes of Health World Wide Web site at URL http://bimas.dcrt.nih.gov. See also the website of: SYFPEITHI: An Internet Database for MHC Ligands and Peptide Motifs (access via http://www.uni-tuebingen.de/uni/kxi/ or http://134.2.96.221/scripts/hlaserver.dll/EpPredict.htm. Methods for determining HLA class II peptides and making substitutions thereto are also known (e.g. Strominger and Wucherpfennig (PCT/US96/03182)).

For example, several predicted HLA binding peptides for the OY-TES-1 polypeptide (SEQ ID NO:24) are listed in Table 10 below. These peptides were predicted using the algorithm of Parker et al (J. Immunol. 152:163, 1994). The binding peptides having the top scores for several HLA molecules are reported. The score for each peptide corresponds to an estimated half time of dissociation of a peptide from an HLA molecule.

TABLE 10
Predicted HLA binding peptides in OY-TES-1 (SEQ ID NO: 24)

| amino acids OF | | Binding score |
|---|---|---|
| SEQ ID NO: 24 | HLA molecule | (t_{1/2} disassociation) |
| 321-330 | A1 | 563 |
| 476-484 | A1 | 125 |
| 478-487 | A1 | 125 |
| 11-20 | A_0201 | 182 |
| 280-288 | A_0201 | 174 |
| 324-333 | A_0201 | 158 |
| 48-56 | A_0201 | 138 |
| 280-288 | A_0205 | 214 |
| 42-50 | A24 | 360 |
| 347-356 | A24 | 360 |
| 428-437 | A24 | 240 |
| 433-441 | A24 | 120 |
| 419-417 | A68.1 | 600 |
| 359-347 | A68.1 | 200 |
| 163-171 | A68.1 | 100 |
| 170-179 | B14 | 600 |
| 501-510 | B14 | 400 |
| 502-510 | B14 | 180 |
| 176-185 | B60 | 176 |
| 41-49 | B60 | 160 |
| 482-490 | B62 | 125 |
| 426-434 | B_2702 | 1000 |
| 528-539 | B_2702 | 300 |
| 507-515 | B_2702 | 200 |
| 109-117 | B_2702 | 200 |
| 308-316 | B_2702 | 200 |
| 528-539 | B_2705 | 10000 |
| 366-374 | B_2705 | 6000 |
| 426-434 | B_2705 | 5000 |
| 68-76 | B_2705 | 2000 |
| 1-9 | B_2705 | 2000 |
| 308-317 | B_2705 | 2000 |
| 365-374 | B_2705 | 2000 |
| 203-212 | B_2705 | 2000 |
| 392-401 | B_2705 | 2000 |
| 119-127 | B_2705 | 1800 |
| 322-330 | B_4403 | 720 |
| 299-307 | B_4403 | 600 |
| 293-302 | B_4403 | 600 |
| 100-109 | B_4403 | 180 |
| 145-153 | B_5101 | 400 |
| 323-332 | B_5101 | 400 |
| 319-327 | B_5101 | 260 |
| 519-527 | B_5101 | 242 |
| 344-343 | B_5101 | 242 |
| 151-160 | B_5101 | 220 |
| 253-261 | B_5101 | 220 |
| 192-201 | B_5101 | 220 |
| 344-353 | B_5102 | 1210 |
| 151-160 | B_5102 | 660 |
| 323-332 | B_5102 | 660 |
| 145-153 | B_5102 | 484 |
| 6-15 | B_5102 | 440 |
| 264-272 | B_5102 | 400 |
| 406-414 | B_5102 | 363 |
| 372-380 | Cw_0301 | 200 |
| 482-490 | Cw_0301 | 150 |
| 41-49 | Cw_0301 | 150 |
| 41-50 | Cw_0301 | 150 |
| 7-16 | Cw_0401 | 480 |
| 319-328 | Cw_0401 | 264 |
| 42-50 | Cw_0401 | 240 |
| 164-172 | Cw_0401 | 240 |
| 347-356 | Cw_0401 | 240 |
| 533-542 | Cw_0401 | 240 |

### Example 8

### Identification of the Portion of a Cancer Associated Polypeptide Encoding an Antigen

To determine if the cancer associated antigens isolated as described above can provoke a cytolytic T lymphocyte response, the following method is performed. CTL clones are generated by stimulating the peripheral blood lymphocytes (PBLs) of a patient (or mouse) with autologous normal cells transfected with one of the clones encoding a cancer associated antigen polypeptide or with irradiated PBLs loaded with synthetic peptides corresponding to the putative protein and matching the consensus for the appropriate HLA class I molecule (as described above) to localize an antigenic peptide within the cancer associated antigen clone (see, e.g., Knuth et al., Proc. Natl. Acad. Sci. USA 81:3511-3515, 1984; van der Bruggen et al., Eur. J. Immunol. 24:3038-3043, 1994). These CTL clones are screened for specificity against COS cells transfected with the cancer associated antigen clone and autologous HLA alleles as described by Brichard et al. (Eur. J. Immunol. 26:224-230, 1996). CTL recognition of a cancer associated antigen is determined by measuring release of TNF from the cytolytic T lymphocyte or by ⁵¹Cr release assay (Herin et al., Int. J. Cancer 39:390-396, 1987). If a CTL clone specifically recognizes a transfected COS cell, then shorter fragments of the cancer associated antigen clone transfected in that COS cell are tested to identify the region of the gene that encodes the peptide. Fragments of the cancer associated antigen clone are prepared by exonuclease III digestion or other standard molecular biology methods. Synthetic peptides are prepared to confirm the exact sequence of the antigen.

Optionally, shorter fragments of cancer associated antigen cDNAs are generated by PCR. Shorter fragments are used to provoke TNF release or ⁵¹Cr release as above.

Synthetic peptides corresponding to portions of the shortest fragment of the cancer associated antigen clone which provokes TNF release are prepared. Progressively shorter peptides are synthesized to determine the optimal cancer associated antigen tumor rejection antigen peptides for a given HLA molecule.

A similar method is performed to determine if the cancer associated antigen contains one or more HLA class II peptides recognized by T cells. One can search the sequence of the cancer associated antigen polypeptides for HLA class II motifs as described above. In contrast to class I peptides, class II peptides are presented by a limited number of cell types. Thus for these experiments, dendritic cells or B cell clones which express HLA class II molecules preferably are used.

TABLE 11
Sequence homologies

SEQ ID NO:9

AL031055, U03904, AB021490.2, AF144629.1, U81385, X94191, X60820, M80939,
M80938, W45845, AA276778, W12845, AA445681, AI174093, AA163016, AA791702,
AA125123, AA793827, AA198900, AA003763, AI182954, AI597203.1, AA589600,
AA561605, AV323570.1, AI614370.1, AA497714, AV233618.1, AW107276.1,
AA754927, AA510149, W41547, AF039235, AA358918, AA373939, AI916936.1,
AA315994, AA361102, AI440178.1, AI022281, AI743350.1, AA621367, AA598742,
Z25343.

SEQ ID NO:13

NM_004592.1, J05080, AL034558.2, AC011001.2, AF168956.1, AB019001.1,
AF126480.1, AC005142, AB015598.1, AF098161, AF101438.1, AF071506,
AF071527.1, AB011167, U58748, L38477, Z11997, Y00365, U00431, L14429,
L19249, AF003151.2, AP000346.1, AC005135, Z83122.1, L44140.1, X86810,
D64111, L36829, K02587, NM_000117.1, X00654, X68946, AL132979.1,
AC012380.1, AF109719.2, NM_006719.2, NM_002313.3, AB022692.1, NM 006720.1,
AJ242956.1, NM_005691.1, NM_005378.1, AL034408.2, U71084, AJ131148.1,
AF042714, AF076274, AC004005, Z93242, AF018165, AF025480, Z75710.1,
AF005370, AF005654, Z81515.1, Z78416.1, Z72686, Z71584, X59601, L38856,
U33758, D31967, Z48618, Z46259, X59863, X5756r, X17199, Z21611, X07699,
D26114, X03295, X02363, Y00664, Z22572, NM_004606.1, M32092, M13241,
L23114, M73444, L19597, L27631, L09209, D31883, NM_001642.1, X74225.1,
M22580, M23216, M34271, M13228, D21877, H44689, AA027262, AA256873,
AI871457.1, AI950925.1, AA812028, AI431592.1, AA374595, AA437048, AA282462,
N84830, R70432, AI417091.1, AI391464, AI016313, AA548044, AA484436,
AA442000, W52798, W25804, W24038, N49505, AW005182.1, AW002246.1,
AI937788.1, AI937103.1, AL044552.1, AL041478.1, AI696132.1, AL047599.1,
AI499031.1, AI371599, AI368947, AI368871, AI337311, AI334569, AI255139,
AI242685, AA725144, AA693685, AA663606, AA578529, AA315123, AA.203519,
AA167831, AA083111, AA057640, AA203468, W67602, W67601, N95426, R80498,
R79280, R66447, R52824, R33900, AI316495, AA420323, AI173226, AW045177.1,
C80075, AW049212.1, AW045819.1, AW048739.1, C76919, AA254996, C87870,
AA030824, AA561021, AI573843.1, AA030365, W08619, AA416313, AV330281.1,
AA204230, AV277735.1, AV094608.1, AI152993, AV031612.1, AV254814.1,
AV276238.1, AV315661.1, AV274035.1, AV240388.1, AV365468.1, AV374341.1,
AV298568.1, AV330260.1, AV305225.1, AI154346, AV225442.1, AI839392.1,
AV326271.1, AV349170.1, AV247245.1, AA816058, AV257049.1, AV235616.1,
AV322742.1, AV080798.1, AA068921, AV226134.1, AV275975.1, AV248838.1,
W82727, AA217762, D18136, AA789824, AI527541, AI227319, W64508, AA797091,
AA239762, AA823138, AA475558, AA197732, W70850, AI956759.1, AV170718.1,
AI791067.1, AI790663.1, AV125900.1, AV122779.1, AV096937.1, AI648759.1,
AI550256.1, AI463840, AU015737, AU015657, AU014623, AA895398, AA798375,
AA797955, AA793428, C81382, C80585, C80564, AA607305, AA588982, AA571572,
AA560832, AA474262, AA467444, AA413093, AA275567, AA123213, AA120232,
AA111692, W97542, AI842860.1, AA268371, AA031077, AV294981.1, AA833307,
AA472076, AI711096.1, AI709709.1, AI233561, AI180068, AI170909, AI102461,
AI576455.1, AA957106.1, AI008470, AA944954, AI043740, AI175545, H33858,
AW165033.1, AW128282.1, AW019641.1, AI779631.1, AI774248.1, AI773240.1,
AW165272.1, AW096676.1, AW091803.1, AI722622.1, AI722217.1, AI722132.1,
AI721578.1, AU0.62029.1, AI728625.1, AI542408, AI516896, AA605835, C32241.1,
D67546, AV384376.1, AV383428.1, AI947408.1, AI776807.1, AI626286.1,
AI621775.1, AI586631.1, AI545013, C91835, AF020720, AA494586, T88263,
T13720, T04634, R02924.

SEQ ID NO:15

NM_005003.1, X61997, AC002400, AP131865.1, AC002526, D17577, AL023755.5,
AC006972.2, AL034489, Y16789, AC004512, Z74979, L40557, AI648903.1,
AI119292, AW107104.1, AI647845.1, AI119359, AI929857.1, AI849803.1,
AI047860, AI850286.1, AV000514.1, AI046666, AI413300, W89503, AA939621,
AA210326, AA168999, AA185613, AA929298, AA212234, AI152408, AA915295,
AI172915, AA790460, AI930104.1, AA466529, AA032743, AA048160, AA711023,
AV006031.1, AA738875, AA212314, AA197653, AA030594, W56963, AI227424,
AI893356.1, AA271005, AA060580, W42142, AA119387, W79962, AI115431,
AA822668. AA007973, W80240, W13086, W12715, AI877179.1, AA013975, W15883,
AA500928, AA.238199, W85108, AA930915, AA240213, W40680, AI117345,, W76948,
W50674, AI508285, W82342, AA110994, W43957, AA646522, AA437633, AA989964,
AA178192, AA920691, W48200, AV000328.1, AA271056, AV066478.1, D17880,
AA684079, W83559, AV021796.1, AA204050, AA062534, AA033484, AA823129,
AA162638, AV060641.1, AV083684.1, W16252, AV118462.1, R75584, AV091441.1,
AV080619.1, AV056483.1, AV051905.1, AV085492.1, AA207625, AV085512.1,
AV033153.1, AV006258.1, AV062958.1, AV084081.1, AV225927.1, AV152111.1,
AA690970, AA075950, AA082003, AA074065, AA313368, AW007505.1, AI889052.1,
AI598144.1, AI095590, AI017929, AA310175, AA873566, AA868795, AA868719,
AA777631, AA600754, AA476531, AW044220.1, F36T70.1, F25329.1, AI083718,
AA993399, AA935632, AA618021, AA459170, N42432, N33459, AI873496.1,
F24032.1. AI581853.1, AI302612, AI301635, AI285263, AI248971, AI220450,
AI160021, AI128G30, AI032546, AI016702, AI014825, AA9G1682, AA936142,
AA860514, AA833995, AA831482, AA447569, AA417135, AW166336.1, AW073092.1,
AI863340.1, AI520938, AA829435, AA508601, AI815674.1, AI720708.1, AI161342,
AA875886, AA983159, AA587029, AA689457, AI469794.1, AA729752, AA448553,
AA909264, AA868027, AA507444, AA416980, AA886275, AA883939, AA878640,
AI469742, AI281705, AA885865, AW139387.1, AAI52181, AA729741, N35532,
AA723449, N94436. A.A936986, AA917976, AA632370, AAI50058, N35132, N26481.
AA909159, AA639717, AA642285, N35190, AA906828, AI609646.1, AA827419,
AA689510, AA872218, AA136946, F18960.2, N86284, D54530, AI352654, AA152029,
AI525049.1.

SEQ ID NO:17

U06944, U88344, AC0052E1, AC004073, AC005966, U34740, AF056562.1,
AF056561.1, AB026654.1, AL034418.2, AJ011712.1, AC005546, U92322,
AL022721.1, AL021878, AC003058, Z99102, AB003505, AB003504, AE001099,
A5000493, U49391, U60211, Z69902.1, M34652, AI596060.1, AA272122, AA269508.
AI849761.1, AI874995.1, AU024159, AA270426, AU079534.1, AA475684, W15945,
AA590308, AI956733.1, AA518797, AI847841.1, AI852328.1, AA119752,
AV204180.1, AV306904.1, W87234, AV355049.1, AV320983.1, AI882238.1,
AV299331.1, AV033250.1, AV032030.1, AV269232.1, AA170872, AV301048.1,
AV226430.1, AV355108.1, AI663065.1, AV295507.1, AA119300, AA116521,
AI882051.1, AV364043.1, AV224385.1, AV337033.1, AV248698.1,, AW123672.1,
AV282468.1, AV214423.1, AV309192.1, AV151663.1, AV125955.1,, AV106869.1,
AV098772.1, AV077295.1, AV054772.1, AV027590.1, AV014774.1, AV013646.1,
AV214005.1, AV066448.1, AV026120.1, AV009189.1, AV336813A, AV335057.1,
AV331939.1, AV269945.1, AV227030.1, AV161933.1, AV130365.1,, AV125700.1,
AV121841.1, AV101405.1, AV096440.1, AV089404.1, AV076355.1, AV069807.1,
AV066548.1, AV0542B3.1, AV061514.1, AV055563.1, AV051G13.1, AV006337.1,
AI481849, AI948435.1, AI912734.1, AI796967.1, AI740869.1, AI498701.1,
AI197956, AI197904, AI003139, AA887501, AA191594, W86389, N64011, H99275,
AI985257.1, AA346169, AA219621, AI282275, AA191083, AA779217, D61002;
H05031, H81379, R20553, R41899. AI864073.1, AA992514, D29534, W92008,
AA393678, W92009.

SEQ ID NO:23

D16200.1, AF085884.1, D16203.1, G23540.1, D17574.1, D17573.1, AC018363.6,
AF113672.1, AL117419.1, Y15994.1, AF148693.1, AE003584.1, AC002460.1,
AC004716.1, A1033504.3, X52027.1, D85605.1, D86959.1, AB002804.1,
AA864327.1, AA824340.1, AA496077.1, AI125678.1, AI028208.1, AI126598.1,
AA406076.1, AI769240.1, AI575049.1, AI333870.1, AI333624.1, AI141116.1,
AI131223.1, AI091519.1, AI122673.1, AA709069.1, AA443593.1, AI147148.1,
AA471063.1, H30251.1, R07741.1, AI138894.1, AI024421.1, AA993247.1,
AW592506.1, AA424694.1, AA405433.1, AA473853.1, H30315.1, AI382680.1,
AI574731.1, R07740.1, AA992230.1, AI326707.1, AA154158.1, AI125674.1,
AW358264.1, AI573445.1, AL042116.1, AV005781.1, AI047576.1, AI553526.1.
AA104771.1, AA065355.1, AI208979.1, AI716721.1, AA621303.1, AV040492.2.

Equivalents

Those skilled in the art will recognize, or be able to ascertain using no more than routine experimentation, many equivalents to the specific embodiments of the invention described herein. Such equivalents are intended to be encompassed by the following claims;

All references disclosed herein are incorporated by reference in their entirety.

We claim:

1. A method of diagnosing a disorder characterized by expression of a cancer associated antigen precursor coded for by a nucleic acid molecule, comprising:
contacting a biological sample isolated from a subject with an agent that specifically binds to the nucleic acid molecule, an expression product thereof, or a fragment of an expression product thereof complexed with a MHC molecule, wherein the nucleic acid molecule is a NA Group 1 molecule, and
determining the interaction between the agent and the nucleic acid molecule or the expression product as a determination of the disorder.

2. The method of claim 1, wherein the agent is selected from the group consisting of
(a) a nucleic acid molecule comprising NA group 1 molecules or a fragment thereof,
(b) a nucleic acid molecule comprising NA group 3 molecules or a fragment thereof,
(c) a nucleic acid molecule comprising NA group 5 molecules or a fragment thereof, is (d) an antibody that binds to an expression product of NA group 1 molecules,
(e) an antibody that binds to an expression product of NA group 3 molecules,
(f) an antibody that binds to an expression product of NA group 5 molecules, t
(g) an agent that binds to a complex of a MHC molecule and a fragment of an expression product of a NA group 1 molecule,
(h) an agent that binds to a complex of a MHC molecule and a fragment of an expression product of a NA group 3 molecule, and
(i) an agent that binds to a complex of a MHC molecule and a fragment of an expression product of a NA group 5 molecule.

3. The method of claim 1, wherein the disorder is characterized by expression of a plurality of cancer associated antigen precursors and wherein the agent is a plurality of agents, each of which is specific for a different cancer associated antigen precursor, and wherein said plurality of agents is at least 2, at least 3, at least 4, at least 5, at least 6, at least 7, or at least 8, at least 9 or at least 10 such agents.

4. The method of claims 1-3, wherein the agent is specific for a cancer associated antigen precursor that is a fibrosarcoma cancer associated antigen precursor.

5. The method of claim 2, wherein the NA group 1 molecule is SEQ ID NO:23.

6. A method for determining regression, progression or onset of a condition characterized by expression of abnormal levels of a protein encoded by a nucleic acid molecule that is a NA Group 1 molecule, comprising
monitoring a sample, from a patient who has or is suspected of having the condition, for a parameter selected from the group consisting of
(i) the protein,
(ii) a peptide derived from the protein,
(iii) an antibody which selectively binds the protein or peptide, and
(iv) cytolytic T cells specific for a complex of the peptide derived from the protein and an MHC molecule,
as a determination of regression, progression or onset of said condition.

7. The method of claim 6, wherein the sample is a body fluid, a body effuision or a tissue.

8. The method of claim 6, wherein the step of monitoring comprises contacting the sample with a detectable agent selected from the group consisting of
(a) an antibody which selectively binds the protein of (i), or the peptide of (ii),
(b) a protein or peptide which binds the antibody of (iii), and
(c) a cell which presents the complex of the peptide and MHC molecule of (iv).

9. The method of claim 8, wherein the antibody, the protein, the peptide or the cell is labeled with a radioactive label or an enzyme.

10. The method of claim 6, comprising assaying the sample for the peptide.

11. The method of claim 6, wherein the nucleic acid molecule is a NA Group 3 molecule.

12. The method of claim 6, wherein the nucleic acid molecule is a NA Group 5 molecule.

13. The method of claim 6, wherein the protein is a plurality of proteins, the parameter is a plurality of parameters, each of the plurality of parameters being specific for a different of the plurality of proteins, at least one of which is a cancer associated protein encoded by a NA Group 1 molecule.

14. The method of claim 6, wherein the NA Group 1 molecule is SEQ ID NO:23.

15. A pharmaceutical preparation comprising
an agent which when administered to the subject enriches selectively the presence of complexes of a MHC molecule and a cancer associated antigen, and
a pharmaceutically acceptable carrier, wherein the cancer associated antigen is a fragment of a cancer associated antigen precursor encoded by a nucleic acid molecule comprising a NA Group 1 molecule.

16. The pharmaceutical preparation of claim 15, wherein the agent comprises a plurality of agents, each of which enriches selectively in the subject complexes of a MHC molecule and a different cancer associated antigen, wherein at least one of the cancer associated antigens is encoded by a NA Group 1 molecule.

17. The pharmaceutical preparation of claim 16, wherein the plurality is at least two, at least three, at least four or at least 5 different such agents.

18. The pharmaceutical preparation of claim 15, wherein the nucleic acid molecule is a NA Group 3 nucleic acid molecule.

19. The pharmaceutical preparation of claim 15, wherein the agent is selected from the group consisting of
(1) an isolated polypeptide comprising the cancer associated antigen, or a functional variant thereof,
(2) an isolated nucleic acid operably linked to a promoter for expressing the isolated polypeptide, or functional variant thereof,
(3) a host cell expressing the isolated polypeptide, or functional variant thereof, and
(4) isolated complexes of the polypeptide, or functional variant thereof, and a MHC molecule.

20. The pharmaceutical preparation of claim 15, wherein the NA Group 1 molecule is SEQ ID NO:23.

21. The pharmaceutical preparation of claims 15-20, further comprising an adjuvant.

22. The pharmaceutical preparation of claim 15, wherein the agent is a cell expressing an isolated polypeptide comprising the cancer associated antigen or a functional variant thereof, and wherein the cell is nonproliferative.

23. The pharmaceutical preparation of claim 15, wherein the agent is a cell expressing an isolated polypeptide comprising the cancer associated antigen or a functional variant thereof, and wherein the cell expresses a MHC molecule that binds the polypeptide.

24. The pharmaceutical preparation of claim 15, wherein the agent is at least two, at least three, at least four or at least five different polypeptides, each coding for a different cancer associated antigen or functional variant thereof, wherein at least one of the cancer associated antigens is encoded by a NA Group 1 molecule.

25. The pharmaceutical preparation of claim 15, wherein the agent is a PP Group 2 polypeptide.

26. The pharmaceutical preparation of claim 15, wherein the agent is a PP Group 3 polypeptide or a PP Group 4 polypeptide.

27. The pharmaceutical preparation of claim 23, wherein the cell expresses one or both of the polypeptide and the MHC molecule recombinantly.

28. The pharmaceutical preparation of claim 23, wherein the cell is nonproliferative.

29. A composition comprising
an isolated agent that binds selectively a PP Group 1 polypeptide.

30. The composition of claim 29, wherein the PP Group 1 polypeptide is SEQ ID NO:24.

31. The composition of matter of claim 26, wherein the agent binds selectively a PP Group 2 polypeptide.

32. The composition of matter of claim 29, wherein the agent binds selectively a PP Group 3 polypeptide.

33. The composition of matter of claim 29, wherein the agent binds selectively a PP Group 4 polypeptide.

34. The composition of matter of claim 29, wherein the agent binds selectively a PP Group 5 polypeptide.

35. The composition of claims 29-34, wherein the agent is a plurality of different agents that bind selectively at least two, at least three, at least four, or at least five different such polypeptides.

36. The composition of claims 29-34, wherein the agent is an antibody.

37. The composition of claim 35, wherein the agent is an antibody.

38. A composition of matter comprising
a conjugate of the agent of claims 29-34 and a therapeutic or diagnostic agent.

39. A composition of matter comprising
a conjugate of the agent of claim 35 and a therapeutic or diagnostic agent.

40. The composition of matter of claim 38, wherein the conjugate is of the agent and a therapeutic or diagnostic that is a toxin.

41. A pharmaceutical composition comprising an isolated nucleic acid molecule selected from the group consisting of NA Group 1 molecules and NA Group 2 molecules, and a pharmaceutically acceptable carrier.

42. The pharmaceutical composition of claim 41, wherein the NA Group 1 molecule is SEQ ID NO:23.

43. The pharmaceutical composition of claim 41, wherein the isolated nucleic acid molecule comprises a NA Group 3 or NA Group 4 molecule.

44. The pharmaceutical composition of claim 41, wherein the isolated nucleic acid molecule comprises at least two isolated nucleic acid molecules coding for two different, polypeptides, each polypeptide comprising a different cancer associated antigen.

45. The pharmaceutical composition of claims 41-44 further comprising an expression vector with a promoter operably linked to the isolated nucleic acid molecule.

46. The pharmaceutical composition of claims 4144 further comprising a host cell recombinantly expressing the isolated nucleic acid molecule.

47. A pharmaceutical composition comprising
an isolated polypeptide comprising a PP Group 1 or a PP Group 2 polypeptide, and
a pharmaceutically acceptable carrier.

48. The pharmaceutical composition of claim 47, wherein the PP Group 1 polypeptide is SEQ ID NO:24.

49. The pharmaceutical composition of claim 47, wherein the isolated polypeptide comprises a PP Group 3 or a PP Group 4 polypeptide.

50. The pharmaceutical composition of claim 47, wherein the isolated polypeptide comprises at least two different polypeptides, each comprising a different cancer associated antigen.

51. The pharmaceutical composition of claim 47, wherein the isolated polypeptides are PP Group 3 polypeptides or MHC binding fragments thereof.

52. The pharmaceutical composition of claim 47, wherein the isolated polypeptides are PP Group 5 polypeptides or MHC binding fragments thereof.

53. The pharmaceutical composition of claims 47-52, further comprising an adjuvant.

54. An isolated nucleic acid molecule comprising a NA Group 3 molecule.

55. An isolated nucleic acid molecule comprising a NA Group 4 molecule.

56. An isolated nucleic acid molecule selected from the group consisting of
(a) a fragment of a nucleic acid molecule having a nucleotide sequence selected from the group consisting of nucleotide sequences set forth as SEQ ID NOs. 9, 13, 15, 17, 19, and 23, of sufficient length to represent a sequence unique within the mouse or human genomes, and identifying a nucleic acid encoding a cancer associated antigen precursor,
(b) complements of (a),
provided that the fragment includes a sequence of contiguous nucleotides which is not identical to any sequence selected from the sequence group consisting of
(1) sequences having the GenBank accession numbers of Table 11,
(2) complements of (1), and
(3) fragments of (1) and (2).

57. The isolated nucleic acid molecule of claim 56, wherein the sequence of contiguous nucleotides is selected from the group consisting of:
(1) at least two contiguous nucleotides nonidentical to the sequence group,
(2) at least three contiguous nucleotides nonidentical to the sequence group,
(3) at least four contiguous nucleotides nonidentical to the sequence group,
(4) at least five contiguous nucleotides nonidentical to the sequence group,
(5) at least six contiguous nucleotides nonidentical to the sequence group,
(6) at least seven contiguous nucleotides nonidentical to the sequence group.

58. The isolated nucleic acid molecule of claim 56, wherein the fragment has a size selected from the group consisting of at least: 8 nucleotides, 10 nucleotides, 12 nucleotides, 14 nucleotides, 16 nucleotides, 18 nucleotides, 20, nucleotides, 22 nucleotides, 24 nucleotides, 26 nucleotides, 28 nucleotides, 30 nucleotides, 50 nucleotides, 75 nucleotides, 100 nucleotides, and 200 nucleotides.

59. The isolated nucleic acid molecule of claim 50, wherein the molecule encodes a polypeptide which, or a fragment of which, binds a MHC receptor or an antibody.

60. An expression vector comprising an isolated nucleic acid molecule of any of claims 54-59 operably linked to a promoter.

61. An expression vector-comprising a nucleic acid operably linked to a promoter, wherein the nucleic acid is a NA Group 2 molecule.

62. An expression vector comprising a NA Group 1 or Group 2 molecule and a nucleic acid encoding a MHC molecule.

63. The expression vector of claim 62, wherein the NA Group 1 molecule is SEQ ID NO:23.

64. A host cell transformed or transfected with an expression vector of claim 60.

65. A host cell transformed or transfected with an expression vector of any of claims 61-63.

66. A host cell transformed or transfected with an expression vector of claim 60 and further comprising a nucleic acid encoding a MHC molecule.

67. A host cell transformed or transfected with an expression vector of claim 61 and further comprising a nucleic acid encoding a MHC molecule.

68. An isolated polypeptide encoded by the isolated nucleic acid molecule of claim 54 or claim 55.

69. A fragment of the polypeptide of claim 68 which is immunogenic.

70. The fragment of claim 69, wherein the fragment, or a portion of the fragment, binds a MHC molecule or an antibody.

71. An isolated fragment of a cancer associated antigen precursor which, or portion of which, binds a MHC molecule or an antibody, wherein the precursor is encoded by a nucleic acid molecule that is a NA Group 1 molecule.

72. The isolated fragment of claim 71, wherein the NA Group 1 molecule is SEQ ID NO:23.

73. The fragment of claim 71-, wherein the fragment is part of a complex with a MHC molecule.

74. The fragment of claim 73, wherein the fragment is between 8 and 12 amino acids in length.

75. An isolated polypeptide comprising a fragment of the polypeptide of claim 68 of sufficient length to represent a sequence unique within the mouse or human genomes and identifying a polypeptide that is a cancer associated antigen precursor.

76. A kit for detecting the presence of the expression of a cancer associated antigen precursor comprising
a pair of isolated nucleic acid molecules each of which consists essentially of a molecule selected from the group consisting of (a) a 12-32 nucleotide contiguous segment of the nucleotide sequence of any of the NA Group 1 molecules and (b) complements of (a), wherein the contiguous segments are nonoverlapping.

77. The kit of claim 76, wherein the NA Group 1 molecule is SEQ ID NO:23.

78. The kit of claim 76, wherein the pair of isolated nucleic acid molecules is constructed and arranged to selectively amplify an isolated nucleic acid molecule that is a NA Group 3 molecule.

79. A method for treating a subject with a disorder characterized by expression of a cancer associated antigen precursor, comprising
administering to the subject an amount of an agent, which enriches selectively in the subject the presence of complexes of a MHC molecule and a cancer associated antigen, effective to ameliorate the disorder, wherein the cancer associated antigen is a fragment of a cancer associated antigen precursor encoded by a nucleic acid molecule selected from the group consisting of
(a) a nucleic acid molecule comprising NA Group 1 molecules,
(b) a nucleic acid molecule comprising NA Group 3 molecules,
(c) a nucleic acid molecule comprising NA Group 5 molecules.

80. The method of claim 79, wherein the NA Group 1 molecule is SEQ ID NO:23.

81. The method of claim 79, wherein the disorder is characterized by expression of a plurality of cancer associated antigen precursors and wherein the agent is a plurality of agents, each of which enriches selectively in the subject the presence of complexes of a MHC molecule and a different cancer associated antigen, wherein at least one of the cancer associated antigens is encoded by a NA Group 1 molecule.

82. The method of claim 81, wherein the plurality is at least 2, at least 3, at least 4, or at least 5 such agents.

83. The method of claims 79-82, wherein the agent is an isolated polypeptide selected from the group consisting of PP Group 1, PP Group 2, PP Group 3, PP Group 4 and PP Group 5.

84. The method of claims 79-82, wherein the disorder is cancer.

85. The method of claims 83, wherein the disorder is cancer.

86. A method for treating a subject having a condition characterized by expression of a cancer associated antigen precursor in cells of the subject, comprising:
(i) removing an immunoreactive cell containing sample from the subject,
(ii) contacting the immunoreactive cell containing sample to the host cell under conditions favoring production of cytolytic T cells against a cancer associated antigen which is a fragment of the precursor,
(iii) introducing the cytolytic T cells to the subject in an amount effective to lyse cells which express the human cancer associated antigen, wherein the host cell is transformed or transfected with an expression vector comprising an isolated nucleic acid molecule operably linked to a promoter, the isolated nucleic acid molecule being selected from the group of nucleic acid molecules consisting of NA Group 1 molecules, NA Group 2 molecules, NA Group 3 molecules, NA Group 4 molecules, and NA Group 5 molecules.

87. The method of claim 86, wherein the NA Group 1 molecule is SEQ ID NO:23.

88. The method of claim 86, wherein the host cell recombinantly expresses a MHC molecule which binds the cancer associated antigen.

89. The method of claim 86, wherein the host cell endogenously expresses a MHC molecule which binds the cancer associated antigen.

90. A method for treating a subject having a condition characterized by expression of a cancer associated antigen precursor in cells of the subject, comprising:
(i) identifying a nucleic acid molecule expressed by the cells associated with said condition, wherein said nucleic acid molecule is a NA Group 1 molecule;
(ii) transfecting a host cell with a nucleic acid selected from the group consisting of (a) the nucleic acid molecule identified, (b) a fragment of the nucleic acid identified which includes a segment coding for a cancer associated antigen, (c) deletions, substitutions or additions to (a) or (b), and (d) degenerates of (a), (b), or (c);
(iii) culturing said transfected host cells to express the transfected nucleic acid molecule, and;
(iv) introducing an amount of said host cells or an extract thereof to the subject effective to increase an immune response against the cells of the subject associated with the condition.

91. The method of claim 90, wherein the NA Group 1 molecule is SEQ ID NO:23.

92. The method of claim 90, further comprising identifying a MHC molecule which presents a portion of an expression product of the nucleic acid molecule, wherein the host cell expresses the same MHC molecule as identified and wherein the host cell presents a MHC binding portion of the expression product of the nucleic acid molecule.

93. The method of claim 90, wherein the immune response comprises a B-cell response or a T-cell response.

94. The method of claim 93, wherein the response is a T-cell response which comprises generation of cytolytic T-cells specific for the host cells presenting the portion of the expression product of the nucleic acid molecule or cells of the subject expressing the cancer associated antigen.

95. The method of claim 90, wherein the nucleic acid molecule is a NA Group 3 molecule.

96. The method of any of claims 90-92, further comprising treating the host cells to render them non-proliferative.

97. A method for treating or diagnosing or monitoring a subject having a condition characterized by expression of an abnormal amount of a protein encoded by a nucleic acid molecule that is a NA Group 1 molecule, comprising
administering to the subject an antibody which specifically binds to the protein or a peptide derived therefrom, the antibody being coupled to a therapeutically useful agent, in an amount effective to treat the condition.

98. The method of claim 97, wherein the NA Group 1 molecule is SEQ ID NO:23.

99. The method of claim 97, wherein the antibody is a monoclonal antibody.

100. The method of claim 99, wherein the monoclonal antibody is a chimeric antibody or a humanized antibody.

101. A method for treating a condition characterized by expression in a subject of abnormal amounts of a protein encoded by a nucleic acid molecule that is a NA Group 1 nucleic acid molecule, comprising
administering to a subject a pharmaceutical composition of any one of claims 13-25 and 37-47 in an amount effective to prevent, delay the onset of, or inhibit the condition in the subject.

102. The method of claim 101, wherein the condition is cancer.

103. The method of claim 101, further comprising first identifying that the subject expresses in a tissue abnormal amounts of the protein.

104. The method of claim 102, further comprising first identifying that the subject expresses in a tissue abnormal amounts of the protein.

105. A method for treating a subject having a condition characterized by expression of abnormal amounts of a protein encoded by a nucleic acid molecule that is a NA Group 1 nucleic acid molecule, comprising
(i) identifying cells from the subject which express abnormal amounts of the protein;
(ii) isolating a sample of the cells;
(iii) cultivating the cells, and
(iv) introducing the cells to the subject in an amount effective to provoke an immune response against the cells.

106. The method of claim 105, wherein the NA Group 1 molecule is SEQ ID NO:23.

107. The method of claim 105, further comprising rendering the cells non-proliferative, prior to introducing them to the subject.

108. A method for treating a pathological cell condition characterized by aberrant expression of a protein encoded by a nucleic acid molecule that is a NA Group 1 nucleic acid molecule, comprising
administering to a subject in need thereof an effective amount of an agent which inhibits the expression or activity of the protein.

109. The method of claim 108, wherein the NA Group 1 molecule is SEQ ID NO:23.

110. The method of claim 108, wherein the agent is an inhibiting antibody which selectively binds to the protein and wherein the antibody is a monoclonal antibody, a chimeric antibody or a humanized antibody.

111. The method of claim 108, wherein the agent is an antisense nucleic acid molecule which selectively binds to the nucleic acid molecule which encodes the protein.

112. The method of claim 108, wherein the nucleic acid molecule is a NA Group 3 nucleic acid molecule.

113. A composition of matter useful in stimulating an immune response to a plurality of a proteins encoded by nucleic acid molecules that are NA Group 1 molecules, comprising
a plurality of peptides derived from the amino acid sequences of the proteins, wherein the peptides bind to one or more MHC molecules presented on the surface of the cells which express an abnormal amount of the protein.

114. The composition of matter of claim 113, wherein the NA Group 1 molecule is SEQ ID NO:23.

115. The composition of matter of claim 113, wherein at least a portion of the plurality of peptides bind to MHC molecules and elicit a cytolytic response thereto.

116. The composition of matter of claim 115, further comprising an adjuvant.

117. The composition of matter of claim 116, wherein said adjuvant is a saponin, GM-CSF, or an interleukin.

118. The composition of matter of claim 113, further comprising at least one peptide useful in stimulating an immune response to at least one protein which is not encoded by nucleic
